(19) United States
(12) Patent Application Publication
(10) Pub. No.: US 2007/0224626 A1
(43) Pub. Date: Sep. 27, 2007

(54) Comprehensive DNA methylation profiling in a human cancer genome identifies novel epigenetic targets

(75) Inventors: Jeffrey A. Jeddeloh, Oakville, MO (US); Jared Ordway, St. Louis, MO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Orion Genomics LLC, St. Louis, MO (US)

(21) Appl. No.: 11/716,084

(22) Filed: Mar. 8, 2007

Related U.S. Application Data
(60) Provisional application No. 60/781,518, filed on Mar. 9, 2006.

(51) Int. Cl. C12Q 1/68; G06F 19/00

(52) U.S. Cl. 435/6; 702/20

(57) ABSTRACT

The present invention provides DNA marker sequences that are differentially methylated in samples from normal individuals and individuals with brain cancer. The invention further provides methods of identifying differentially methylated DNA marker sequences and their use the detection and diagnosis of gliomas.

### CROSS-REFERENCE TO RELATED PATENT APPLICATIONS

The present application claims benefit of priority to U.S. Provisional Patent Application No. 60/781,518, filed Mar. 9, 2006, which is incorporated by reference in its entirety.

### BACKGROUND OF THE INVENTION

Identifying molecular differences that distinguish tumor tissue from normal tissue is a current topic area of intense interest. Although tumor genomes display only a limited number of primary sequence differences from the nearly isogenic normal tissues in proximity to them, a large number of molecular differences exist. In particular, the spectrum of sequences that normal and tumor genomes specify and mark as silent chromatin have been used as “epigenetic signatures” to molecularly discriminate these cells.

Disruption of normal gene regulation is important for carcinogenesis resulting in loss, or gain of genetic function. The molecular events that underlie this altered regulation include point-mutations, and macro-mutations such as deletion, amplification or genomic rearrangement (e.g. translocation), that can result in more complex interactions when regulatory genes are affected. Recently, the importance of epigenetic perturbation of gene regulation in the form of changes in chromatin structure has begun to be more fully appreciated. In the context of cancer, inappropriate chromatin packaging of genes can lead to gene silencing, or in some cases, ectopic gene expression.

Cytosine methylation is a chemically stable mark that may establish, or follow as a consequence of, the packaging of a particular region into silent chromatin. Therefore, identification of aberrant genomic DNA methylation associated with carcinogenesis identifies loci that are important for disease progression.

Mammalian DNA methylation patterns that transmit cellular silencing signals are mitotically maintained with 96-99% fidelity. This lies in stark contrast with the primary sequence which is maintained with fidelity over 99.9999%. Recent studies have highlighted the role of the environment, for example through dietary folate metabolism, in maintaining DNA methylation and gene silencing, suggesting a mechanism underlies a predisposition for cancer.

Several cancer therapies targeting the maintenance of cytosine methylation and silencing states are in human clinical trials. Currently, the therapies target either the DNA methylation machinery, or the histone modification machinery. This machinery works synergistically to maintain gene silencing. While such therapies are very promising, the clinical success rates achieved thus far have been similar to more conventional chemotherapies. Therefore, selecting patients for such epigenetic therapies or measuring their success may require an understanding and characterization of the sequences affected by epigenetic perturbation in particular diseases.

Finding and characterizing the genomic loci capable of driving carcinogenesis from the epigenetic perspective, as well as those capable of serving as clinically meaningful disease or therapy-specific markers is a pressing need.

### BRIEF SUMMARY OF THE INVENTION

The present invention provides methods for determining the methylation status of an individual. In some embodiments, the methods comprise:

obtaining a biological sample from an individual; and

determining the methylation status of at least one cytosine within a DNA region in a sample from the individual where the DNA region is selected from the group consisting of SEQ ID NO: 40, 41, 42, 43, 44, 45, 46, 47, 48, and 49.

In some embodiments, the determining step comprises determining the methylation status of at least one cytosine in the DNA region corresponding to a nucleotide in a marker, wherein the marker is selected from the group consisting of SEQ ID NO: 30, 31, 32, 33, 34, 35, 36, 37, 38, and 39.

In some embodiments, the determining step comprises determining the methylation status of the entire marker.

In some embodiments, the sample is from brain tissue or cerebral spinal fluid.

In some embodiments, the methylation status of at least one cytosine is compared to the methylation status of a control locus. In some embodiments, the control locus is an endogenous control. In some embodiments, the control locus is an exogenous control.

In some embodiments, the determining step comprises determining the methylation status of at least one cytosine in at least two DNA regions.

In some embodiments, the methods comprise:

a) determining the methylation status of at least one cytosine within a DNA region in a sample from the individual where the DNA region is selected from the group consisting of SEQ ID NO: 40, 41, 42, 43, 44, 45, 46, 47, 48, and 49;

b) comparing the methylation status of the at least one cytosine to a threshold value for the marker, wherein the threshold value distinguishes between individuals with and without brain cancer, wherein the comparison of the methylation status to the threshold value is predictive of the presence or absence of brain cancer in the individual.

In some embodiments, the determining step comprises determining the methylation status of at least one cytosine in the DNA region corresponding to a nucleotide in a marker, wherein the marker is selected from the group consisting of SEQ ID NO: 30, 31, 32, 33, 34, 35, 36, 37, 38, and 39.

In some embodiments, the determining step comprises determining the methylation status of the entire marker.

In some embodiments, the sample is from the brain or cerebral spinal fluid.

In some embodiments, the methylation status of at least one marker from the list is compared to the methylation value of a control locus. In some embodiments, the control locus is an endogenous control. In some embodiments, the control locus is an exogenous control.

In some embodiments, the determining step comprises determining the methylation status of at least one cytosine from at least two DNA regions.

The present invention also provides computer implemented methods for determining the presence or absence of brain cancer in an individual. In some embodiments, the methods comprise:

receiving, at a host computer, a methylation value representing the methylation status of at least one cytosine within a DNA region in a sample from the individual where the DNA region is selected from the group consisting of SEQ ID NO: 40, 41, 42, 43, 44, 45, 46, 47, 48, and 49; and

comparing, in the host computer, the methylation value to a threshold value, wherein the threshold value distinguishes between individuals with and without brain cancer, wherein the comparison of the methylation value to the threshold value is predictive of the presence or absence of brain cancer in the individual.

In some embodiments, the receiving step comprises receiving at least two methylation values, the two methylation values representing the methylation status of at least one cytosine marker from two different DNA regions; and

the comparing step comprises comparing the methylation values to one or more threshold value(s) wherein the threshold value distinguishes between individuals with and without brain cancer, wherein the comparison of the methylation value to the threshold value is predictive of the presence or absence of brain cancer in the individual.

The present invention also provides a computer program product for determining the presence or absence of brain cancer in an individual. In some embodiments, the computer readable products comprise:

a computer readable medium encoded with program code, the program code including:
program code for receiving a methylation value representing the methylation status of at least one cytosine within a DNA region in a sample from the individual where the DNA region is selected from the group consisting of SEQ ID NO: 40, 41, 42, 43, 44, 45, 46, 47, 48, and 49; and
program code for comparing the methylation value to a threshold value, wherein the threshold value distinguishes between individuals with and without brain cancer, wherein the comparison of the methylation value to the threshold value is predictive of the presence or absence of brain cancer in the individual.

The present invention also provides kits for determining the methylation status of at least one marker. In some embodiments, the kits comprise:

a pair of polynucleotides capable of specifically amplifying from human genomic DNA at least a portion of a DNA region where the DNA region is selected from the group consisting of SEQ ID NO: 40, 41, 42, 43, 44, 45, 46, 47, 48, and 49; and

a methylation-dependent and/or methylation sensitive restriction enzyme and/or sodium bisulfite.

In some embodiments, the pair of polynucleotides are capable of specifically amplifying from human genomic DNA a marker selected from the group consisting of SEQ ID NOs: 30, 31, 32, 33, 34, 35, 36, 37, 38, and 39. In some embodiments, the kit comprises at least two pairs of polynucleotides, wherein each pair is capable of specifically amplifying from human genomic DNA at least a portion of a different DNA region. In some embodiments, the kit further comprises a detectably labeled polynucleotide probe that specifically detects the amplified marker in a real time amplification reaction.

In some embodiments, the kits comprise:

sodium bisulfite, primers and adapters for whole genome amplification, and polynucleotides to quantify the presence of the converted methylated and or the converted unmethylated sequence of at least one cytosine from a DNA region that is selected from the group consisting of SEQ ID NO: 40, 41, 42, 43, 44, 45, 46, 47, 48, and 49.

In some embodiments, the kits comprise:

a methylation sensing restriction enzymes, primers and adapters for whole genome amplification, and polynucleotides to quantify the number of copies of at least a portion of a DNA region where the DNA region is selected from the group consisting of SEQ ID NO: 40, 41, 42, 43, 44, 45, 46, 47, 48, and 49.

In some embodiments, the kits comprise:

a methylation sensing binding moiety and polynucleotides to quantify the number of copies of at least a portion of a DNA region where the DNA region is selected from the group consisting of SEQ ID NO: 40, 41, 42, 43, 44, 45, 46, 47, 48, and 49.

The present invention also provides a method for determining the presence or absence of brain cancer in an individual, wherein the method comprises:

a) measuring the amount of IRX3 RNA or IRX3 protein in a sample from the individual;

b) comparing the amount to a threshold value for IRX3 RNA or IRX3 protein, wherein the threshold value distinguishes between individuals with and without brain cancer, wherein if the amount of IRX3 RNA is measured, the amount is compared to a threshold value for IRX3 RNA, and if the amount of IRX3 protein is measured, the amount is compared to a threshold value for IRX3 protein, and wherein the comparison of the amount to the threshold value is predictive of the presence or absence of brain cancer in the individual. In some embodiments, the sample is from the brain or cerebral spinal fluid.

The present invention also provides computer implemented methods for determining the presence or absence of brain cancer in an individual. In some embodiments, the methods comprise:

receiving, at a host computer, an RNA or protein expression value representing the level of IRX3 RNA or protein in a sample from the individual; and

comparing, in the host computer, the expression value to a threshold value, wherein the threshold value distinguishes between individuals with and without brain cancer, wherein the comparison of the expression value to the threshold value is predictive of the presence or absence of brain cancer in the individual.

Definitions

“Methylation” refers to cytosine methylation at positions C5 or N4 of cytosine, the N6 position of adenine or other types of nucleic acid methylation. In vitro amplified DNA is unmethylated because in vitro DNA amplification methods do not retain the methylation pattern of the amplification template. However, “unmethylated DNA” or “methylated DNA” can also refer to amplified DNA whose original template was methylated or methylated, respectively.

A “methylation profile” refers to a set of data representing the methylation states of one or more loci within a molecule of DNA from e.g., the genome of an individual or cells or tissues from an individual. The profile can indicate the methylation state of every base in an individual, can comprise information regarding a subset of the base pairs (e.g., the methylation state of specific restriction enzyme recognition sequence) in a genome, or can comprise information regarding regional methylation density of each locus.

“Methylation status” refers to the presence, absence and/or quantity of methylation at a particular nucleotide, or nucleotides within a portion of DNA. The methylation status of a particular DNA sequence (e.g., a DNA marker or DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.” A methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of methylation status. This is of particular use when it is desirable to compare the methylation status of a sequence in a sample to a threshold value.

A “methylation-dependent restriction enzyme” refers to a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC). For example, McrBC's recognition sequence is 5′ RmC (N40-3000) RmC 3′ where “R” is a purine and “mC” is a methylated cytosine and “N40-3000” indicates the distance between the two RmC half sites for which a restriction event has been observed. McrBC generally cuts close to one half-site or the other, but cleavage positions are typically distributed over several base pairs, approximately 30 base pairs from the methylated base. McrBC sometimes cuts 3′ of both half sites, sometimes 5′ of both half sites, and sometimes between the two sites. Exemplary methylation-dependent restriction enzymes include, e.g., McrBC (see, e.g., U.S. Pat. No. 5,405,760), McrA, MrrA, DpnI, BisI and GlaI. One of skill in the art will appreciate that any methylation-dependent restriction enzyme, including homologs and orthologs of the restriction enzymes described herein, is also suitable for use in the present invention.

A “methylation-sensitive restriction enzyme” refers to a restriction enzyme that cleaves DNA at or in proximity to an unmethylated recognition sequence but does not cleave at or in proximity to the same sequence when the recognition sequence is methylated. Exemplary methylation-sensitive restriction enzymes are described in, e.g., McClelland et al., Nucleic Acids Res. 22(17):3640-59 (1994) and http://rebase.neb.com. Suitable methylation-sensitive restriction enzymes that do not cleave DNA at or near their recognition sequence when a cytosine within the recognition sequence is methylated at position C⁵ include, e.g., Aat II, Aci I, Acl I, Age I, Alu I, Asc I, Ase I, AsiS I, Bbe I, BsaA I, BsaH I, BsiE I, BsiW I, BsrF I, BssH II, BssK I, BstB I, BstN I, BstU I, Cla I, Eae I, Eag I, Fau I, Fse I, Hha I, HinP1 I, HinC II, Hpa II, Hpy99 I, HpyCH4 IV, Kas I, Mbo I, Mlu I, MapA1 I, Msp I, Nae I, Nar I, Not I, Pml I, Pst I, Pvu I, Rsr II, Sac II, Sap I, Sau3A I, Sfl I, Sfo I, SgrA I, Sma I, SnaB I, Tsc I, Xma I, and Zra I. Suitable methylation-sensitive restriction enzymes that do not cleave DNA at or near their recognition sequence when an adenosine within the recognition sequence is methylated at position N⁶ include, e.g., Mbo I. One of skill in the art will appreciate that any methylation-sensitive restriction enzyme, including homologs and orthologs of the restriction enzymes described herein, is also suitable for use in the present invention. One of skill in the art will further appreciate that a methylation-sensitive restriction enzyme that fails to cut in the presence of methylation of a cytosine at or near its recognition sequence may be insensitive to the presence of methylation of an adenosine at or near its recognition sequence. Likewise, a methylation-sensitive restriction enzyme that fails to cut in the presence of methylation of an adenosine at or near its recognition sequence may be insensitive to the presence of methylation of a cytosine at or near its recognition sequence. For example, Sau3AI is sensitive (i.e., fails to cut) to the presence of a methylated cytosine at or near its recognition sequence, but is insensitive (i.e., cuts) to the presence of a methylated adenosine at or near its recognition sequence. One of skill in the art will also appreciate that some methylation-sensitive restriction enzymes are blocked by methylation of bases on one or both strands of DNA encompassing of their recognition sequence, while other methylation-sensitive restriction enzymes are blocked only by methylation on both strands, but can cut if a recognition site is hemi-methylated.

A “threshold value that distinguishes between individuals with and without” a particular disease refers to a value or range of values of a particular measurement that can be used to distinguish between samples from individuals with the disease and samples without the disease. Ideally, there is a threshold value or values that absolutely distinguishes between the two groups (i.e., values from the diseased group are always on one side (e.g., higher) of the threshold value and values from the healthy, non-diseased group are on the other side (e.g., lower) of the threshold value). However, in many instances, threshold values do not absolutely distinguish between diseased and non-diseased samples (for example, when there is some overlap of values generated from diseased and non-diseased samples).

The phrase “corresponding to a nucleotide in a marker” refers to a nucleotide in a DNA region that aligns with the same nucleotide (e.g., a cytosine) in a marker sequence. Generally, as described herein, marker sequences are subsequences of (i.e., have 100% identity with) the DNA regions. Sequence alignments can be performed using any BLAST including BLAST 2.2 algorithm with default parameters, described in Altschul et al., Nuc. Acids Res. 25:3389 3402 (1977) and Altschul et al., J. Mol. Biol. 215:403 410 (1990), respectively.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

As used herein, the terms “nucleic acid,” “polynucleotide” and “oligonucleotide” refer to nucleic acid regions, nucleic acid segments, primers, probes, amplicons and oligomer fragments. The terms are not limited by length and are generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA.

A nucleic acid, polynucleotide or oligonucleotide can comprise, for example, phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, polynucleotide or oligonucleotide can comprise the five biologically occurring bases (adenine, guanine, thymine, cytosine and uracil) and/or bases other than the five biologically occurring bases. For example, a polynucleotide of the invention can contain one or more modified, non-standard, or derivatized base moieties, including, but not limited to, N⁶-methyl-adenine, N⁶-tert-butyl-benzyl-adenine, imidazole, substituted imidazoles, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, uracil-5-oxyacetic acidmethylester, 3-(3-amino-3-N2-carboxypropyl) uracil, (acp3)w, 2,6-diaminopurine, and 5-propynyl pyrimidine. Other examples of modified, non-standard, or derivatized base moieties may be found in U.S. Pat. Nos. 6,001,611; 5,955,589; 5,844,106; 5,789,562; 5,750,343; 5,728,525; and 5,679,785.

Furthermore, a nucleic acid, polynucleotide or oligonucleotide can comprise one or more modified sugar moieties including, but not limited to, arabinose, 2-fluoroarabinose, xylulose, and a hexose.

### BRIEF DESCRIPTION OF THE DRAWINGS

FIG. 1 illustrates array analysis procedure and theoretical results from the methylation profiling technology described in the present invention. A schematic of three array probes (X, Y, and Z) arranged along a chromosome is shown. If the DNA near feature Z is heavily methylated (vertical bars) approximately half of those methylated CG dinucleotides will be half-sites for McrBC (R^{m}CG). Sheared and size-selected genomic DNA was labeled with Cy5 (black), while McrBC treated DNA was labeled with Cy3 (grey). There are 13 black fragments and 13 grey fragments as a consequence of the mass normalization prior to target synthesis. Fragments which contain two R^{m}CG sites have been depleted from the Cy3-labeled population due to the action of McrBC, while unmethylated fragments are enriched by mass normalization. The two color array hybridization results are depicted as white circles for unmethylated, grey for intermediate or adjacent methylation, or black for densely methylated. The relative signal from the two colors is indicated as a log₂ ratio.

FIG. 2 illustrates a representative genome close-up of the locus analysis results. A graphical representation of the transcription start site and 5′ structure of one predicted gene is indicated (A). The bar graph (B) indicates the relative local density of purine-CG sequences within this region. The relative position of the DNA microarray feature that reported DNA methylation at this locus is indicated by (C). PCR primers were selected to amplify the region indicated by (D). The horizontal line (E) represents the 8 Kb sequence (4 Kb upstream and 4 Kb downstream of the DNA sequence represented by the microarray feature (C)) capable of reporting DNA methylation in the microarray-based discovery experiment.

FIG. 3 illustrates the methylation profile of oligodendroglioma derived cell line LN-18 (CRL-2610). Each of 21,294 60mer probes were mapped onto the human genome (NCBIv35) by BLAST and is depicted as a vertical line from the Watson (above the line) or Crick (below the line) DNA strand. Areas devoid of probes represent the centromeres, NOR or 5S gene clusters. DNA methylation from the LN-18 genome is measured by the UT/T ratio (see text for details) depicted as white (unmethylated), grey (intermediately methylated) to black (densely methylated).

FIG. 4 (A-C) illustrates the independent accuracy analysis and suggests quantitative capacity within array analysis. FIG. 4(A) illustrates a scatter plot of the average methylation density determined by bisulphite sequencing obtained from the 11 statistically significant loci plotted versus the normalized log₂ (UT-T) for each feature. The R² value obtained reflects the result of a linear correlation analysis. 2 of the 11 were considered to be discordant with the regression line. FIG. 4(B) illustrates bar charts depicting high resolution cytosine methylation analysis from loci including the points i, ii, and iii. Each of the charts indicate the position of each CG analyzed within each window on the x-axis, and the average relative methylation occupancy for greater than 10 clones at each site within the locus on the y-axis. The ANOVA corrected signal log ratio for each locus is indicated, along with the multiple testing correction p-value intervals the measurements fell within (inset). The position the array probe within the interval is denoted by the black bar in each window. FIG. 4(C) illustrates a scatter-plot of methylation values for 84 genomic loci. Signal ratios from the hybridization results are plotted against the change in cycle threshold (delta Ct) measured by enzyme digestion followed by quantitative PCR. The data are categorized into four quadrants: In clockwise order, false negatives, methylated predictions, false positives and unmethylated predictions. Methylated and unmethylated features are those for which both assays agree. A cut-off of 0.5 cycles is used. The data points are colored according to their significance level: FDR=open circle, Holm=filled circle, grey points are not significant.

FIG. 5 illustrates differentially methylated loci classify brain tissues (cancer and normal) and cell lines at a molecular level. Results from two independent qPCR methylation assays at 10 genomic loci are depicted. Black cells indicate substantial DNA methylation at the genomic locus (ΔCt>>2). White cells indicate little detectable methylation at the locus (ΔCt<1). Grey cells indicate an intermediate amount of methylation (1<ΔCt<2).

FIG. 6 illustrates a high resolution analysis of IRX3 hypermethylation, which reveals an exonic rather than promoter location. FIG. 6(A) illustrates a Gbrowse view of the NCBIv35 genome build with the ENSEMBL_{—}36 annotation depicted along with the LN-18 microarray measurements of local DNA methylation. The arrow scale at the top denotes the bp position along the chromosome. The first data track contains a grey bar representing the IRX3 locus, the arrow depicts its direction of transcription. The second track is the splicing model of the transcript as determined by ENSEMBL_{—}36. The black portions on the track represent the exons. The third data track (RCG) depicts the relative abundance of methylateable McrBC recognition sites within a defined 1 Kb genomic window. The fourth track (OGHA) denotes the positions of the two array features (60mer probes). The fifth track depicts a scaled black bar chart of the ANOVA corrected array measurements for the two features' Log₂ (UT-T); the actual measurements along with their P-values are indicated below each feature. Positive log ratios indicate methylation and negative log ratios indicate its relative absence. FIG. 6(B) illustrates bisulphite sequencing results from analysis of the LN-18 methylation pattern surrounding the array feature depicted as a scatter plot. The X-axis depicts the relative position of each base pair within the interval. The average methylation occupancy at each CG is represented as an open circle. The area under the dashed line between points represents the methylation density. Black arrows denote the position of HhaI restriction sites in the interval (one was outside the sequenced region). The filled circles represent methylation occupancies of CGs at HhaI restriction sites within the interval. The grey bar is the position of the array probe.

FIG. 7 illustrates that the IRX3 exonic CGI is hypermethylated in brain cancer cell lines and tumors but not normal brain cancer samples. Representative SYBR green quantitative PCR kinetic reaction profiles are depicted for nine templates following four enzymatic digestion and PCR treatments. The top row is brain cancer cell lines. The middle row is normal brain samples. The bottom row consists of two primary astrocytoma and one glioblastoma (GBM) sample. Within each profile the four treatments of enzyme digestion are indicated by Roman numerals: I=mock treatment, II=McrBC treatment, III=HhaI treatment, and IV=double digest (HhaI+McrBC). The inset pie-charts reflect the interpretation of the methylation status of the molecular population based upon the average profile data. Black=proportion with uniform HhaI methylation, White=proportion with low to no methylation, Hatched=proportion intermediately methylated, Light grey=proportion refractory to analysis (largely invisible). All the tumor samples had a McrBC conditioned change in Ct>2 cycles; indicating the majority of molecules present contain aberrant methylation relative to the normal samples.

FIG. 8 illustrates IRX3 CGI hypermethylation correlating with over-expression. Panel A depicts Bioanalyzer results obtained following quantification of RT-PCR products in reactions from cDNA libraries prepared from normal brain, tumor and cell line controls. The expression of IRX3 was monitored relative to GAPDH as a benchmark. The normal library was purchased from Invitrogen. Neat, 1:5 and 1:25 reflect the cDNA template dilution factor (into water). Panel B depicts the IRX3 exonic CGI results from FIG. 7. Notice that hypermethylation correlates with over-expression of the RNA, and that there appears to be a relationship between the amount of methylation and the amount of overexpression.

FIGS. 9-13 depict results from clone based bisulphite-sequencing confirmation of each of the different methylation state predictions from FIG. 7. FIG. 9=LN-18, 10=U138MG, 11=U87MG, 12=15084A1 (normal), and 13=JA4CIFNIL (tumor). Each of the figures depicts the results obtained form clone based bisulphate sequencing analysis output in two ways. The bar chart reflects the average methylation occupancy at each CG within the exon of IRX3. The inset panel (upper right) depicts the output from each molecule sequenced. There were more than 25 clones sequenced for each of the genomes depicted. Each methylated C is a black pixel, each unmethylated C is a white pixel. Each line of pixels reflects one molecule sequenced. Note the CpG depicted in the inset are NOT spaced according to their actual distance. Like the results in FIG. 7, the tumor is most like the U87MG cell line.

### DETAILED DESCRIPTION

I. Introduction

DNA methylation is a stable epigenetic signal associated with gene silencing, and establishing a quantitative measurement of DNA methylation to detect disease is a useful diagnostic tool, especially in cancer. Several DNA methylation profiling approaches have been developed (Lippman et al., Nature, 430, 471-76; Weber et al., Nat. Genet., 37, 853-62; Gitan, Genome Res., 12, 158-64; Adorjan et al., Nucleic Acids Res., 30, e21). Yet few have demonstrated the ability to identify both methylated and unmethylated sequences with statistical confidence, and fewer yet, have the capacity to monitor the entire genome.

CpG Islands (CGI) have been characterized as CG rich genomic sequences that do not show CG depletion; they are often in association with genes (Cross and Bird, Curr Opin Genet Dev., 5, 309-14, for review see Fazzari and Greally, Nat. Rev. Genet., 5, 446-55). CGI are generally thought to be devoid of methylation, which accounts for the absence of CG depletion which is driven by the mutagenesis of methylation over evolutionary time. Aberrant cytosine methylation of CGI has been associated with inactivation of tumor suppressor genes in human cancers, consequently the DNA methylation status of CGI is of immense interest.

Changes in DNA methylation typically occur over regions with multiple CG dinucleotides, which are methylated in concert. Thus, regions of methylated sequence rather than single bases may be the most important unit of information in the epigenome.

LN-18 Epigenetic Landscape

Our analysis of the LN-18 genome revealed more than 4000 methylated loci, by design the majority of which were associated with the transcription start sites of human genes. CpG islands (CGI) were disproportionately unmethylated in agreement with previous reports (for review see Fazzari et al., Nat. Rev. Genet. 5, 446-55); however, far more were hypermethylated than anticipated if both intermediate and dense methylation are considered. A conservative estimate of the CGI hypermethylation is revealed by the CGI features reporting dense methylation, which were 2% of the total CGI features. However, since approximately half of the features with intermediate array ratios were also methylated within the feature, upwards of 19% (2%+17%) of the CGI in this genome are methylated. This unexpected finding reflects the more extreme epigenomic representation displayed by cancer derived cell lines.

The CGIs associated with transcriptional start sites (TSS) were most often associated with unmethylated and intermediate array ratios. The importance of the unmethylated status of the loci is relevant, since half of these intermediate measurements will likely represent unmethylated features with adjacent methylated DNA. Further, many more TSS are susceptible to the spread of silencing from these proximally methylated elements. The use of ultra-high density microarrays with much higher feature densities results in more accurate mapping of methylation changes. Because a given feature seemed to be capable of detecting adjacent methylation, the quantitative value obtained from any probe represents a complex output. Signal from a feature provides not only data regarding the density of methylation surrounding the feature, but also information about its chromosomal context.

Identification of Novel Targets of Epigenetic Modification

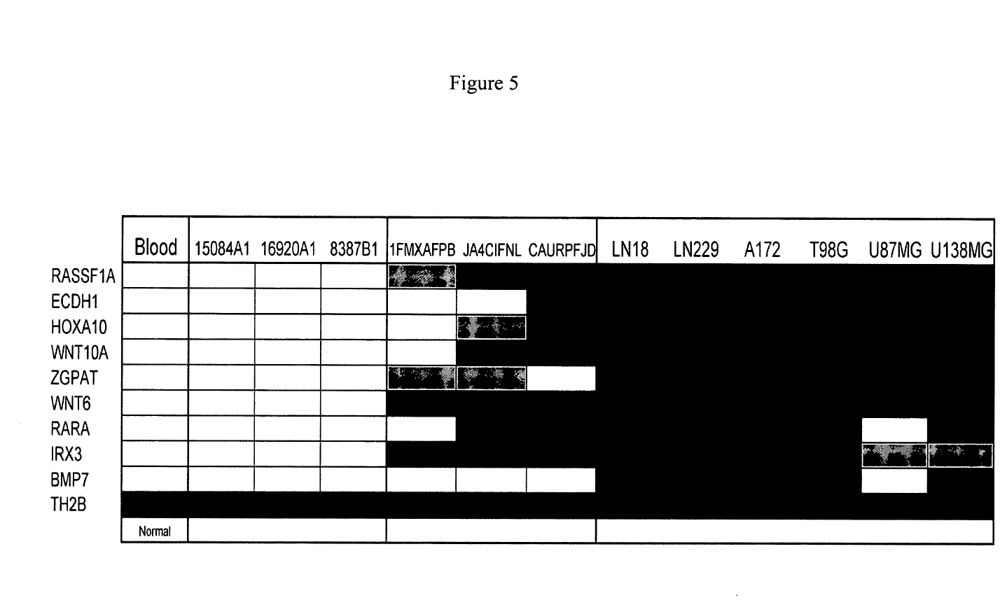

Several targets of DNA hypermethylation were identified in the course of this characterization. We have independently confirmed that the testis specific histone 2B (TH2B) locus is methylated in at least 7 non-testes tissues (brain, cervix, ovary, lung, colon, breast, and lymphocytes), suggesting conservation of epigenetic regulation from rodents to humans (FIG. 5); see also Choi et al., DNA Cell Biol., 15, 495-504. This finding makes the locus useful for a positive control.

Finding that 6 of the 8 differentially methylated loci identified were novel hypermethylation targets suggests that the full number of genomic loci susceptible to epigenetic modification is large. Four of these genes have been implicated in normal brain development (IRX3 (see Bellefroid et al., Embo J., 17, 191-203, BMP7 (see Furuata et al., Development, 124, 2203-12), WNT6, and WNT10A (Kelly et al., Dev Biol. 158, 113-21)). Finally, RARalpha is a member of a gene family that includes other genes subject to hypermethylation in cancer (Cote and Momparler, Anticancer Drugs, 8, 56-61).

Using a unique microarray platform for cytosine methylation profiling (See Appn. No. 10/606,502), the DNA methylation landscape of the human genome was monitored at more than 21,000 sites, including 79% of the annotated transcriptional start sites (TSS). Analysis of an oligodendroglioma derived cell line LN-18 revealed more than 4,000 methylated TSS. The gene-centric analysis indicated a complex pattern of DNA methylation exists along each autosome, with a trend of increasing density approaching the telomeres, where 2% of CpG islands (CGI) were densely methylated and 17% had significant levels of methylation, whether or not they corresponded to a TSS. Substantial independent verification, obtained from 95 loci, suggested that this approach is capable of large scale detection of cytosine methylation with an accuracy approaching 90%. In addition, we detected large genomic domains that are also susceptible to DNA methylation reinforced inactivation, such as the HOX cluster on chromosome 7 (CH7). Extrapolation from the data suggests that more than 2,000 genomic loci may be susceptible to methylation and associated inactivation, and most have yet to be identified. Six new targets of epigenetic inactivation (IRX3, WNT10A, WNT6, RARalpha, BMP7, and ZGPAT) were discovered. These targets displayed cell line and tumor specific differential methylation when compared with normal brain samples.

Identification of Hypermethylation of IRX3 Exon

Another aspect of the present invention provides the hypermethylation of the CGI within an IRX3 exon correlating with over-expression of IRX3 in tumor tissues and cell lines relative to normal brain samples. More specifically, there is a hypermethylated region in exon 2 of the IRX3 gene and coincident upregulation of IRX3 transcription in glial-derived primary tumors and tumor cell lines, relative to normal brain. Given the reported functions of IRX (iroquois) family proteins, this finding may have implications for the development of brain cancer (glioma and astrocytoma). IRX family genes encode homeobox transcription factors conserved from nematodes to humans (Burglin, Nucleic Acids Res, 25, 4173-80). In vertebrates, these factors participate in regulation of proneural genes during early neurulation as well as in antero-posterior and dorso-ventral subdivision of the neural plate (see Gomez-Skarmeta and Modolell, Curr Opin Genet Dev, 12, 403-08).

The power of genomic survey to detect biomarkers is best illustrated by the exonic CGI within the IRX3 gene. Current views of the importance of epigenetic regulation in cancer are based upon the belief that the regulatory regions of tumor suppressor genes become hypermethylated and/or that the regulatory regions of oncogenes may become unmethylated, and that these alterations lead to changes in expression. Contrary to this current view, the present invention shows that the hypermethylation of the IRX3 exon was correlated with overexpression of IRX3 in tumor tissues and cell lines when compared to normal brain tissue samples.

II. Methylation Markers

In some embodiments, the presence or absence or quantity of methylation of the chromosomal DNA within a DNA region or portion thereof (e.g., at least one cytosine) selected from SEQ ID Nos: 40, 41, 42, 43, 44, 45, 46, 47, 48, or 49 is detected. Portions of the DNA regions described herein will comprise at least one potential methylation site (i.e., a cytosine) and can generally comprise 2, 3, 4, 5, 10, or more potential methylation sites. In some embodiments, the methylation status of one or more cytosines within a “marker” is detected. Exemplary markers are SEQ ID NOs: 30, 31, 32, 33, 34, 35, 36, 37, 38 and 39, wherein SEQ ID NO:30 is a marker within DNA region SEG ID NO:40, SEQ ID NO:31 is a marker with SEQ ID NO:41, etc. Exemplary primers for amplification of the exemplary markers can be found in the SEQUENCE LISTING and as described in Table 1 of Example 1.

In some embodiments, the methylation of at least one cytosine in more than one DNA region (or portion thereof) is detected. In some embodiments, the methylation status of 1, 2, 3, 4, 5, 6, 7, 8, 9, or 10 of the DNA regions (SEQ ID NOs: 40-49) is determined.

In some embodiments of the invention, the methylation of a DNA region or portion thereof is determined and then normalized (e.g., compared) to the methylation of a control locus. Typically the control locus will have a known, relatively constant, methylation status. For example, the control sequence can be previously determined to have no, some or a high amount of methylation, thereby providing a relative constant value to control for error in detection methods, etc., unrelated to the presence or absence of cancer. In some embodiments, the control locus is endogenous, i.e., is part of the genome of the individual sampled. For example, in mammalian cells, the testes-specific histone 2B gene (hTH2B in human) gene is known to be methylated in all somatic tissues except testes. Alternatively, the control locus can be an exogenous locus, i.e., a DNA sequence spiked into the sample in a known quantity and having a known methylation status. Such exogenous sequences can be methylated in vitro, if desired, using a DNA methylase.

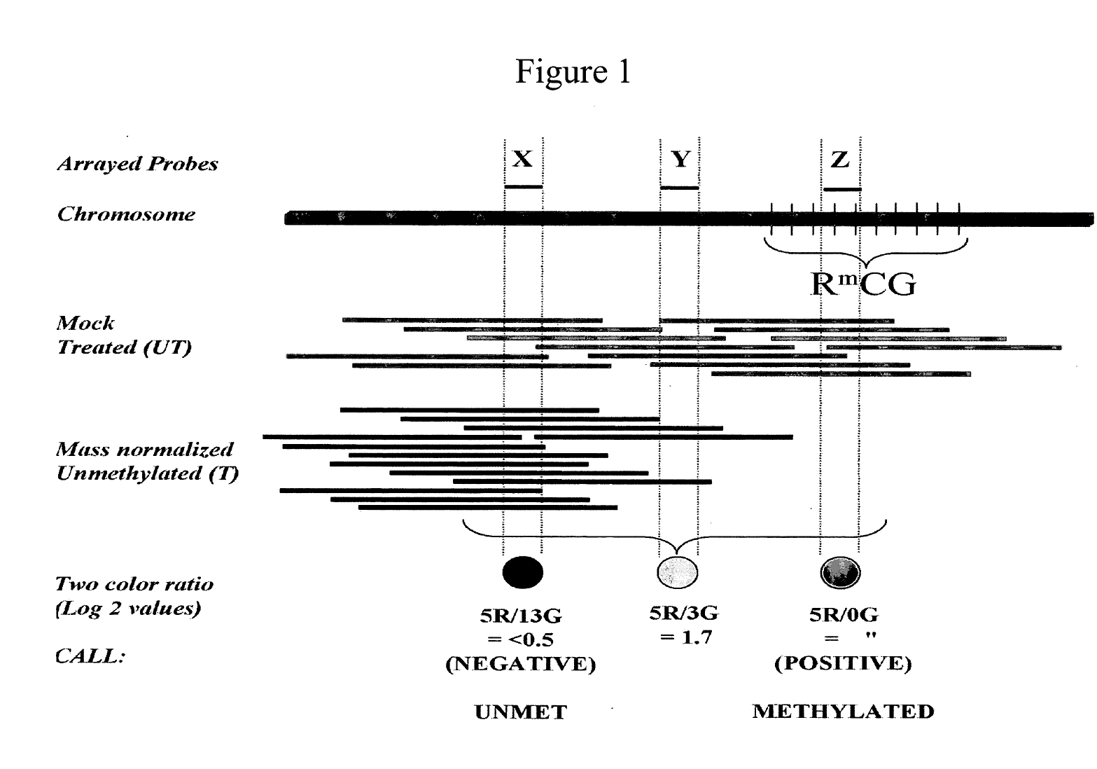

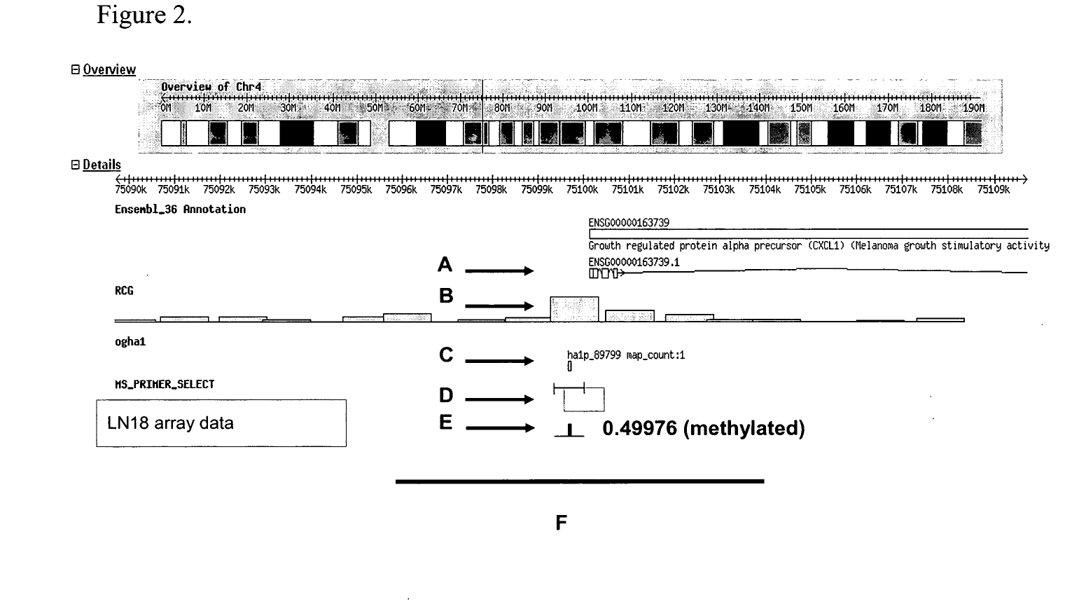

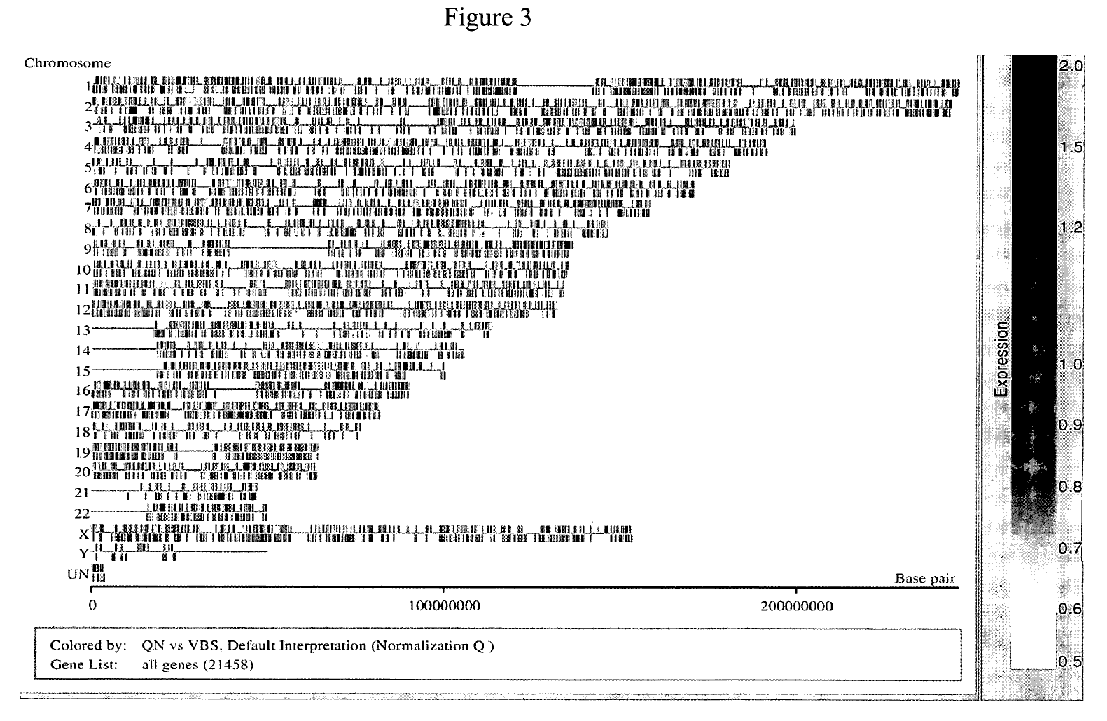

A DNA region comprises a nucleic acid including one or more methylation sites of interest (e.g., a cytosine, a “microarray feature,” or an amplicon amplified from select primers) and flanking nucleic acid sequences (i.e., “wingspan”) of up to 4 kilobases (kb) in either or both of the 3′ or 5′ direction from the amplicon as exemplified in FIG. 1G. This range corresponds to the lengths of DNA fragments obtained by randomly fragmenting the DNA before screening for differential methylation between DNA in two or more samples (e.g., carrying out methods used to initially identify differentially methylated sequences as described in the Examples, below). In some embodiments, the wingspan of the one or more DNA regions is about 0.5 kb, 0.75 kb, 1.0 kb, 1.5 kb, 2.0 kb, 2.5 kb, 3.0 kb, 3.5 kb or 4.0 kb in both 3′ and 5′ directions relative to the sequence represented by the microarray feature.

The methylation sites in a DNA region can reside in non-coding transcriptional control sequences (e.g., promoters, enhancers, etc.) or in coding sequences, including introns and exons of the designated genes listed in Table 1 and in section “SEQUENCE LISTING.” In some embodiments, the methods comprise detecting the methylation status in the promoter regions (e.g., comprising the nucleic acid sequence that is about 1.0 kb, 1.5 kb, 2.0 kb, 2.5 kb, 3.0 kb, 3.5 kb or 4.0 kb 5′ from the transcriptional start site through to the transcriptional start site) of one or more of the genes identified in Tables 1 and in section “SEQUENCE LISTING.”

The DNA regions of the invention also include naturally occurring variants, including for example, variants occurring in different subject populations and variants arising from single nucleotide polymorphisms (SNPs). SNPs encompasses insertions and deletions of varying size and simple sequence repeats, such as dinucleotides and trinucleotide repeats. Variants include nucleic acid sequences from the same DNA region (e.g., as set forth in Table 1 and in section “SEQUENCE LISTING”) sharing at least 90%, 95%, 98%, 99% sequence identity, i.e., having one or more deletions, additions, substitutions, inverted sequences, etc., relative to the DNA regions described herein.

III. Methods for Determining Methylation

Any method for detecting DNA methylation can be used in the methods of the present invention.

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. See, e.g., U.S. Patent Publication No. 2004/0132048. Alternatively, the method can encompass amplifying intact DNA after restriction enzyme digestion, thereby only amplifying DNA that was not cleaved by the restriction enzyme in the area amplified. See, e.g., U.S. patent application Ser. Nos. 10/971,986; 11/071,013; and 10/971,339. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

In some embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

The quantity of methylation of a locus of DNA can be determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample. Such assays are disclosed in, e.g., U.S. patent application Ser. No. 10/971,986.

Kits for the above methods can include, e.g., one or more of methylation-dependent restriction enzymes, methylation-sensitive restriction enzymes, amplification (e.g., PCR) reagents, probes and/or primers.

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol. Biotechnol. 20(2):163-79 (2002). Amplifications may be monitored in “real time.”

Additional methods for detecting DNA methylation can involve genomic sequencing before and after treatment of the DNA with bisulfite. See, e.g., Frommer et al., Proc. Natl. Acad. Sci. USA 89:1827-1831 (1992). When sodium bisulfite is contacted to DNA, unmethylated cytosine is converted to uracil, while methylated cytosine is not modified.

In some embodiments, restriction enzyme digestion of PCR products amplified from bisulfite-converted DNA is used to detect DNA methylation. See, e.g., Sadri & Hornsby, Nucl. Acids Res. 24:5058-5059 (1996); Xiong & Laird, Nucleic Acids Res. 25:2532-2534 (1997).

In some embodiments, a MethyLight assay is used alone or in combination with other methods to detect DNA methylation (see, Eads et al.; Cancer Res. 59:2302-2306 (1999)). Briefly, in the MethyLight process genomic DNA is converted in a sodium bisulfite reaction (the bisulfite process converts unmethylated cytosine residues to uracil). Amplification of a DNA sequence of interest is then performed using PCR primers that hybridize to CpG dinucleotides. By using primers that hybridize only to sequences resulting from bisulfite conversion of unmethylated DNA, (or alternatively to methylated sequences that are not converted) amplification can indicate methylation status of sequences where the primers hybridize. Similarly, the amplification product can be detected with a probe that specifically binds to a sequence resulting from bisulfite treatment of a unmethylated (or methylated) DNA. If desired, both primers and probes can be used to detect methylation status. Thus, kits for use with MethyLight can include sodium bisulfite as well as primers or detectably-labeled probes (including but not limited to Taqman or molecular beacon probes) that distinguish between methylated and unmethylated DNA that have been treated with bisulfite. Other kit components can include, e.g., reagents necessary for amplification of DNA including but not limited to, PCR buffers, deoxynucleotides; and a thermostable polymerase.

In some embodiments, a Ms-SNuPE (Methylation-sensitive Single Nucleotide Primer Extension) reaction is used alone or in combination with other methods to detect DNA methylation (see, Gonzalgo & Jones, Nucleic Acids Res. 25:2529-2531 (1997)). The Ms-SNuPE technique is a quantitative method for assessing methylation differences at specific CpG sites based on bisulfite treatment of DNA, followed by single-nucleotide primer extension (Gonzalgo & Jones, supra). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest.

Typical reagents (e.g., as might be found in a typical Ms-SNuPE-based kit) for Ms-SNuPE analysis can include, but are not limited to: PCR primers for specific gene (or methylation-altered DNA sequence or CpG island); optimized PCR buffers and deoxynucleotides; gel extraction kit; positive control primers; Ms-SNuPE primers for a specific gene; reaction buffer (for the Ms-SNuPE reaction); and detectably-labeled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

In some embodiments, a methylation-specific PCR (“MSP”) reaction is used alone or in combination with other methods to detect DNA methylation. An MSP assay entails initial modification of DNA by sodium bisulfite, converting all unmethylated, but not methylated, cytosines to uracil, and subsequent amplification with primers specific for methylated versus unmethylated DNA. See, Herman et al., Proc. Natl. Acad. Sci. USA 93:9821-9826, (1996); U.S. Pat. No. 5,786,146.

Additional methylation detection methods include, but are not limited to, methylated CpG island amplification (see, Toyota et al., Cancer Res. 59:2307-12 (1999)) and those described in, e.g., U.S. Patent Publication 2005/0069879; Rein, et al. Nucleic Acids Res. 26 (10): 2255-64 (1998); Olek, et al. Nat. Genet. 17(3): 275-6 (1997); and PCT Publication No. WO 00/70090.

IV. Determining Gene and Protein Expression

It is well known that methylation of genomic DNA can affect expression (transcription and/or translation) of nearby gene sequences. Therefore, in some embodiments, the methods include the step of correlating the methylation status of at least one cytosine in a DNA region with the expression of nearby coding sequences, as described in Table 1 and in section “SEQUENCE LISTING.” For example, expression of gene sequences within about 1.0 kb, 1.5 kb, 2.0 kb, 2.5 kb, 3.0 kb, 3.5 kb or 4.0 kb in either the 3′ or 5′ direction from the cytosine of interest in the DNA region can be detected. In some embodiments, the gene or protein expression of a gene in Table 1 and in section “SEQUENCE LISTING” is compared to a control, for example, the methylation status in the DNA region and/or the expression of a nearby gene sequence from a sample from an individual known to be negative for cancer or known to be positive for cancer, or to an expression level that distinguishes between cancer and noncancer states. Such methods, like the methods of detecting methylation described herein, are useful in providing diagnosis, prognosis, etc., of cancer. Methods for measuring transcription and/or translation of a particular gene sequence are well known in the art. See, for example, Ausubel, Current Protocols in Molecular Biology, 1987-2006, John Wiley & Sons; and Sambrook and Russell, Molecular Cloning: A Laboratory Manual, 3rd Edition, 2000, Cold Spring Harbor Laboratory Press.

In some embodiments, the methods further comprise the step of correlating the methylation status and expression of one or more of the gene regions identified in Tables 1 and in section “SEQUENCE LISTING.”

The present invention thus provides for detection of gene (e.g. RNA) and/or protein expression to detect brain cancer. RNA or protein expression from the genomic regions described herein can be compared to a threshold value or otherwise normal expression (i.e., expression for normal, non-cancerous brain tissue) to detect brain cancer. In some embodiments, IRX3 RNA or IRX3 protein is detected and compared to a threshold value or otherwise normal IRX3 brain expression (i.e., expression for normal, non-cancerous brain tissue) to detect brain cancer.

Any method of detecting RNA or protein expression can be used in the methods of the invention. In some embodiments, the presence of cancer is evaluated by determining the level of expression of mRNA encoding a protein of interest. Methods of evaluating RNA expression of a particular gene are well known to those of skill in the art, and include, inter alia, hybridization and amplification based assays.

Direct Hybridization-Based Assays

Methods of detecting and/or quantifying the level of gene transcripts of interest (mRNA or cDNA made therefrom) using nucleic acid hybridization techniques are known to those of skill in the art. For example, one method for evaluating the presence, absence, or quantity of polynucleotides involves a northern blot. Gene expression levels can also be analyzed by techniques known in the art, e.g., dot blotting, in situ hybridization, RNase protection, probing DNA microchip arrays, and the like.

Amplification-Based Assays

In another embodiment, amplification-based assays are used to measure the expression level of a gene of interest. In such an assay, the nucleic acid sequences act as a template in an amplification reaction (e.g., Polymerase Chain Reaction, or PCR). In a quantitative amplification, the amount of amplification product will be proportional to the amount of template in the original sample (e.g., can from a reverse transcription reaction of the target RNA). Comparison to appropriate controls provides a measure of the level of expression of the gene of interest in the sample. Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.). The nucleic acid sequences provided herein are sufficient to enable one of skill to select primers to amplify any portion of the gene anda/ encoded RNA.

In one non-limiting embodiment, a TaqMan™ based assay is used to quantify the cancer-associated polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, literature provided by Perkin-Elmer, e.g., www2.perkin-elmer.com).

Other suitable amplification methods include, but are not limited to, ligase chain reaction (LCR) (see, Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Detection of Protein

Polypeptides encoded by the genes described herein can be detected and/or quantified by any methods known to those of skill in the art from samples as described herein. In some embodiments, antibodies can also be used to detect polypeptides encoded by the genes described herein. Antibodies to these polypeptides can be produced using well known techniques (see, e.g., Harlow & Lane, Antibodies: A Laboratory Manual (1988) and Harlow & Lane, Using Antibodies (1999); Coligan, Current Protocols in Immunology (1991); Goding, Monoclonal Antibodies: Principles and Practice (2d ed. 1986); and Kohler & Milstein, Nature 256:495-497 (1975)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

Once specific antibodies are available, binding interactions with the proteins of interest can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra).

Immunoassays also often use a labeling agent to specifically bind to and label the complex formed by the antibody and antigen. The labeling agent may itself be one of the moieties comprising the antibody/antigen complex. Thus, the labeling agent may be a labeled polypeptide or a labeled antibody that binds the protein of interest. Alternatively, the labeling agent may be a third moiety, such as a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the labeling agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Commonly used assays include noncompetitive assays, e.g., sandwich assays, and competitive assays. In competitive assays, the amount of polypeptide present in the sample is measured indirectly by measuring the amount of a known, added (exogenous) polypeptide of interest displaced (competed away) from an antibody that binds by the unknown polypeptide present in a sample. Commonly used assay formats include immunoblots, which are used to detect and quantify the presence of protein in a sample. Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

V. Cancer Detection

The present markers and methods can be used in the diagnosis, prognosis, classification, prediction of disease risk, detection of recurrence of disease, and selection of treatment of brain cancer. Any stage of progression can be detected, such as primary, metastatic, and recurrent brain cancer. Information regarding numerous types of cancer can be found, e.g., from the American Cancer Society (available on the worldwide web at cancer.org), or from, e.g., Harrison's Principles of Internal Medicine, Kaspar, et al., eds., 16th Edition, 2005, McGraw-Hill, Inc.

The present invention provides methods for determining whether or not a mammal has brain cancer, whether or not a biological sample contains cancerous cells, estimating the likelihood of a mammal developing brain cancer, classifying brain cancer stages, and monitoring the efficacy of anti-cancer treatment in a mammal with brain cancer. Such methods are based on the discovery that cancer cells differentially methylate DNA sequences at the diagnostic DNA regions of the invention. Accordingly, by determining whether or not a cell contains methylated DNA sequences in the DNA regions as described herein, it is possible to determine whether or not the cell is cancerous. Similarly, as described herein, quantification of IRX3 RNA or IRX3 protein levels in brain tissues can be used to determine the presence or absence of brain cancer cells.

In numerous embodiments of the present invention, the presence of methylated nucleotides in the diagnostic DNA regions of the invention is detected in a biological sample, thereby detecting the presence or absence of cancerous cells in the biological sample. In some embodiments, the biological sample comprises a tissue sample from a tissue suspected of containing cancerous cells. Human genomic DNA samples can be obtained by any means known in the art. In cases where a particular phenotype or disease is to be detected, DNA samples should be prepared from a tissue of interest, or as appropriate, from cerebral spinal fluid. For example, DNA can be prepared from biopsy tissue to detect the methylation state of a particular locus associated with cancer. The nucleic acid-containing specimen used for detection of methylated loci (see, e.g., Ausubel et al., Current Protocols in Molecular Biology (1995 supplement)) may be from any source and may be extracted by a variety of techniques such as those described by Ausubel et al., Current Protocols in Molecular Biology (1995) or Sambrook et al., Molecular Cloning, A Laboratory Manual (3rd ed. 2001). Exemplary tissues include, e.g., brain tissue. As appropriate, the tissue or cells can be obtained by any method known in the art including by surgery. In other embodiments, a tissue sample known to contain cancerous cells, e.g., from a tumor, will be analyzed for the presence or quantity of methylation at one or more of the diagnostic markers of the invention to determine information about the brain cancer, e.g., the efficacy of certain treatments, the survival expectancy of the individual, etc. In some embodiments, the methods will be used in conjunction with additional diagnostic methods, e.g., detection of other cancer markers, etc.

The methods of the invention can be used to evaluate individuals known or suspected to have brain cancer or as a routine clinical test, i.e., in an individual not necessarily suspected to have brain cancer. Further diagnostic assays can be performed to confirm the status of cancer in the individual.

Further, the present methods may be used to assess the efficacy of a course of treatment. For example, the efficacy of an anti-cancer treatment can be assessed by monitoring DNA methylation of the marker sequences described herein over time in a mammal having brain cancer. For example, a reduction or absence of methylation in any of the diagnostic markers of the invention in a biological sample taken from a mammal following a treatment, compared to a level in a sample taken from the mammal before, or earlier in, the treatment, indicates efficacious treatment.

The methods detecting brain cancer can comprise the detection of one or more other cancer-associated polynucleotide or polypeptides sequences. Accordingly, detection of methylation of any one or more of the diagnostic markers of the invention can be used either alone, or in combination with other markers, for the diagnosis or prognosis of cancer.

The methods of the present invention can be used to determine the optimal course of treatment in a mammal with cancer. For example, the presence of methylated DNA within any of the diagnostic markers of the invention or an increased quantity of methylation within any of the diagnostic markers of the invention can indicate a reduced survival expectancy of a mammal with brain cancer, thereby indicating a more aggressive treatment for the mammal. In addition, a correlation can be readily established between the presence, absence or quantity of methylation at a diagnostic marker, as described herein, and the relative efficacy of one or another anti-cancer agent. Such analyses can be performed, e.g., retrospectively, i.e., by detecting methylation in one or more of the diagnostic genes in samples taken previously from mammals that have subsequently undergone one or more types of anti-cancer therapy, and correlating the known efficacy of the treatment with the presence, absence or levels of methylation of one or more of the diagnostic markers.

In making a diagnosis, prognosis, risk assessment, classification, detection of recurrence or selection of therapy based on the presence or absence of methylation in at least one of the diagnostic markers, the quantity of methylation may be compared to a threshold value that distinguishes between one diagnosis, prognosis, risk assessment, classification, etc., and another. For example, a threshold value can represent the degree of methylation found at a particular DNA region that adequately distinguishes between brain cancer samples and normal brain biopsy samples with a desired level of sensitivity and specificity. It is understood that a threshold value will likely vary depending on the assays used to measure methylation, but it is also understood that it is a relatively simple matter to determine a threshold value or range by measuring methylation of a DNA sequence in brain and normal samples using the particular desired assay and then determining a value that distinguishes at least a majority of the cancer samples from a majority of non-cancer samples. If methylation of two or more DNA regions is detected, two or more different threshold values (one for each DNA region) will often, but not always, be used.

In some embodiments, the methods comprise recording a diagnosis, prognosis, risk assessment or classification, based on the methylation status determined from an individual. Any type of recordation is contemplated, including electronic recordation, e.g., by a computer.

VI. Kits

This invention also provides kits for the detection and/or quantification of the diagnostic markers of the invention, or expression or methylation thereof using the methods described herein.

For kits for detection of methylation, the kits of the invention can comprise at least one polynucleotide that hybridizes to at least one of the diagnostic marker sequences of the invention and at least one reagent for detection of gene methylation. Reagents for detection of methylation include, e.g., sodium bisulfite, polynucleotides designed to hybridize to sequence that is the product of a marker sequence of the invention if the marker sequence is not methylated (e.g., containing at least one C→U conversion), and/or a methylation-sensitive or methylation-dependent restriction enzyme. The kits can provide solid supports in the form of an assay apparatus that is adapted to use in the assay. The kits may further comprise detectable labels, optionally linked to a polynucleotide, e.g., a probe, in the kit. Other materials useful in the performance of the assays can also be included in the kits, including test tubes, transfer pipettes, and the like. The kits can also include written instructions for the use of one or more of these reagents in any of the assays described herein.

In some embodiments, the kits of the invention comprise one or more reagents as described herein for detecting and/or quantifying a target RNA or protein from a sample. In some embodiments, the reagents are for the specific detection of IRX3 RNA or protein.

VII. Computer-Based Methods

The calculations for the methods described herein can involve computer-based calculations and tools. The tools are advantageously provided in the form of computer programs that are executable by a general purpose computer system (referred to herein as a “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### EXAMPLES

### Example 1

### Identification of Novel Methylated Loci

I. Cell Line, Normal and Tumor Brain Tissue, and Blood Nucleic Acid Samples.

Cell lines LN-18 (glioblastoma), A-172 (glioblastoma), LN-229 (glioblastoma), T-98G (glioblastoma multiforme (GBM)), U-138 MG (astrocytoma) and U-87 MG (astrocytoma) were obtained from American Type Culture Collection (Manassas, Va.) and cultured under supplier's recommendations (http://www.atcc.org/). Histologically normal/non-malignant brain tissue (˜0.4 mg ea) from the cerebrum (right temporal lobe) of three different patients (15084A1, 16920A1, 8387B1) were acquired from Asterand plc. (Detroit, Mich.). Primary tumor samples (two astrocytoma (1FMXAFPB, JA4CIFNL) and one GBM specimen (CAURPFJD)) were acquired from Genomics Collaborative Inc (Cambridge, Mass.). All the tumor samples exhibited greater than 90% neoplastic cellularity. The age range of the normal samples (45, 56, 87) was controlled relative to the tumors (48, 59, 59). Genomic DNA was extracted by the MasterPure DNA Purification kit under manufacturer's recommended conditions (Epicentre Biotechnologies, Madison, Wis.). Male whole blood genomic DNA, representing a pool of peripheral blood samples obtained from approximately six individuals, was obtained from Novagen (Madison, Wis.).

RNA was purified from the tumor tissues and cell lines using the RNeasy Kit from Qiagen (Valencia, Calif.). cDNA from normal brain and astrocytoma tissues was purchased from Invitrogen (Carlsbad, Calif.). cDNA from the cell lines and tumor samples was produced using the oligo dT primers provided in the Superscript III first strand system from Invitrogen (Carlsbad, Calif.) under the manufacturer's recommended conditions.

II. Bisulfite Sequencing.

2 μg LN-18 genomic DNA was bisulfite converted with an EZ DNA Methylation Kit (Zymo Research) using the manufacturer's recommended protocol. Primers and loci selected for validation, along with PCR conditions are indicated in the Supplemental Data. Sequencing and analysis was performed as previously described.

III. Quantitative PCR-Based Validation of DNA Methylation Predictions.

For each analyzed genomic DNA sample, 4 μg of genomic DNA was digested with McrBC (NEB) in 100 μL total volume including 1×NEB2 buffer, 0.1 mg/mL bovine serum albumin, 2 mM GTP and 50 units McrBC overnight at 37° C. In parallel, 4 μg of each genomic DNA sample was mock-treated under identical conditions with the exception that water was substituted for McrBC. Following digestion, enzyme activity was heat inactivated at 65° C. for 20 min. 40 ng McrBC-digested and 40 ng mock-treated DNA were analyzed by quantitative real-time PCR. In all cases, digested and mock-treated templates were analyzed in adjacent wells. Reactions included 1× FailSafe Buffer G (EpiCentre), 5 μM each primer, and 1 unit Taq polymerase (Invitrogen) in 25 μL total volume. Standard quantitative PCR cycling conditions were used with a “hot” plate read of 72° C. for 2 min. The melt curve of each amplicon was calculated within a temperature gradient from 60° C. to 95° C. at 1° C. increments with a 10 sec. hold time for each read. The cycle number at which the McrBC-digested sample crossed the threshold was subtracted from the cycle number at which the mock-treated sample crossed the threshold to determine the deltaCt of each locus. Since McrBC digests only DNA including purine-methylation, thereby decreasing the amplifiable copies of loci containing DNA methylation and increasing the Ct relative to the mock-treated sample, increasing deltaCt values reflect increasing levels of local DNA methylation. All average deltaCt values (UT-McrBC) less than −1 were set to −1. Four condition assays employed the two treatments described above (mock and methylation dependant restriction enzyme (MDRE)), as well as a methylation sensitive restriction enzyme treatment (MSRE, e.g. HhaI (NEB (Beverly, Mass.))) and a double digest treatment (e.g. Hha I+McrBC).

IV. Results

We ran the 25 quantitative PCR assays that detected regional methylation against a panel of genomic DNAs from six brain cancer derived cell lines, as well as pooled male and female peripheral blood which were collected independently from the tumor origin. We employed the CGI in the TH2B gene as a positive control for single copy gene associated methylation detection. Among these loci, 8 were differentially methylated in the majority of cell lines relative to peripheral blood (FIG. 5). Two of these 8 loci were previously demonstrated to be subject to DNA methylation mediated gene silencing; RASSFIA (Hesson et al., Oncogene, 23, 2408-19) and E-Cadherin (CDHI) (Yoshiura, et al., Proc. Natl. Acad. Sci. USA, 92, 7416-19). The remaining six genes that displayed hypermethylation relative to blood, and normal brain tissue have not been reported to be subject to epigenetic silencing. These newly discovered methylated loci were associated with the IRX3, BMP7, WNTIOA, WNT6, RARalpha, and ZGPAT genes. The same panel of loci was differentially methylated in a comparison of primary brain tissue from three apparently normal patients in comparison to primary tumor tissues derived from two astrocytomas and one glioma (GBM).

TABLE 1

| | | Feature | Lprimer | Rprimer | Amplicon | Region |
|---|---|---|---|---|---|---|
| Gene | Feature | ID | ID | ID | ID | ID |
| TH2B | TH2B | | Seq 10 | Seq 20 | Seq 30 | Seq 40 |
| ECDH1 | ha1c_00072 | Seq 1 | Seq 11 | Seq 21 | Seq 31 | Seq 41 |
| RASSF1A | ha1c_00077 | Seq 2 | Seq 12 | Seq 22 | Seq 32 | Seq 42 |
| HOXA10 | ha1p_10831 | Seq 3 | Seq 13 | Seq 23 | Seq 33 | Seq 43 |
| ZGPAT | ha1p_33140 | Seq 4 | Seq 14 | Seq 24 | Seq 34 | Seq 44 |
| BMP7 | ha1p_57533 | Seq 5 | Seq 15 | Seq 25 | Seq 35 | Seq 45 |
| IRX3 | ha1p_58072 | Seq 6 | Seq 16 | Seq 26 | Seq 36 | Seq 46 |
| WNT6 | ha1p_72837 | Seq 7 | Seq 17 | Seq 27 | Seq 37 | Seq 47 |
| WNT10A | ha1p_72985 | Seq 8 | Seq 18 | Seq 28 | Seq 38 | Seq 48 |
| RARAlpha | ha1p_94854 | Seq 9 | Seq 19 | Seq 29 | Seq 39 | Seq 49 |

### Example 2

### IRX3 CGI Methylation is Correlated with IRX3 Overexpression

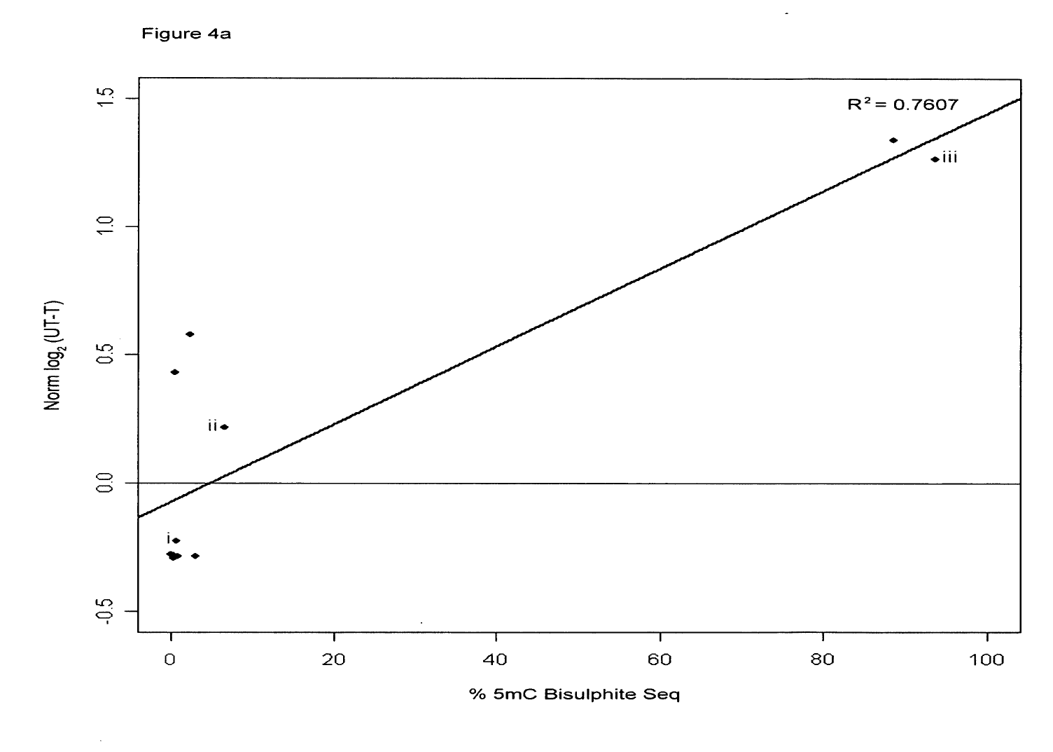

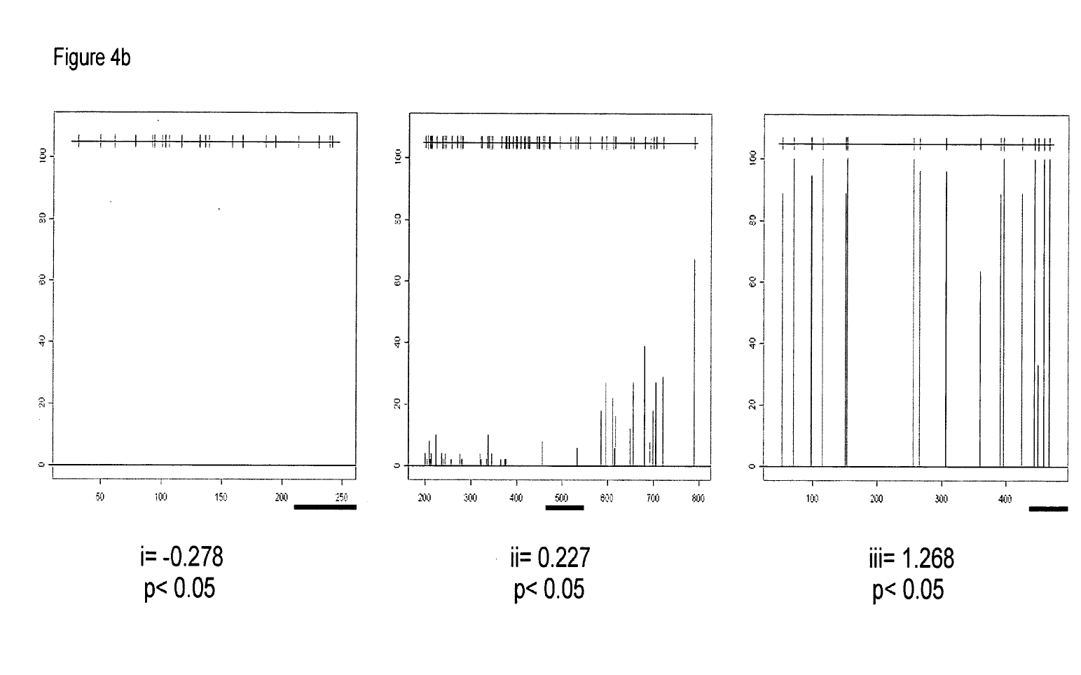

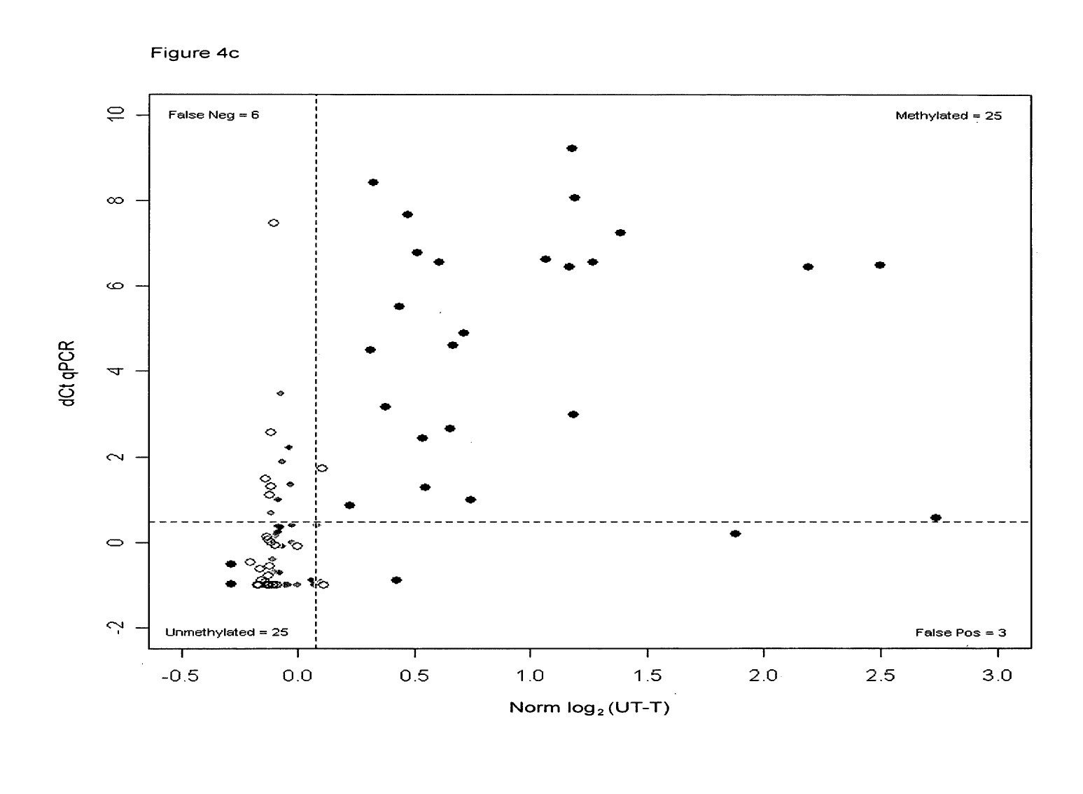

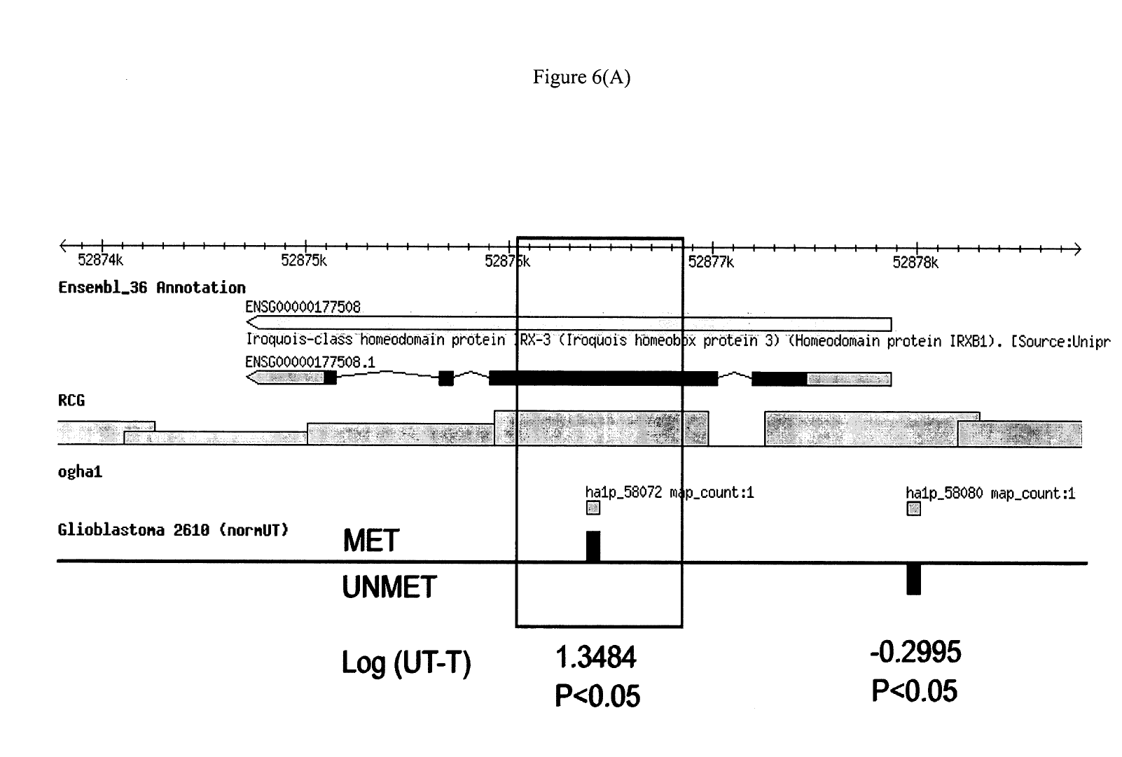

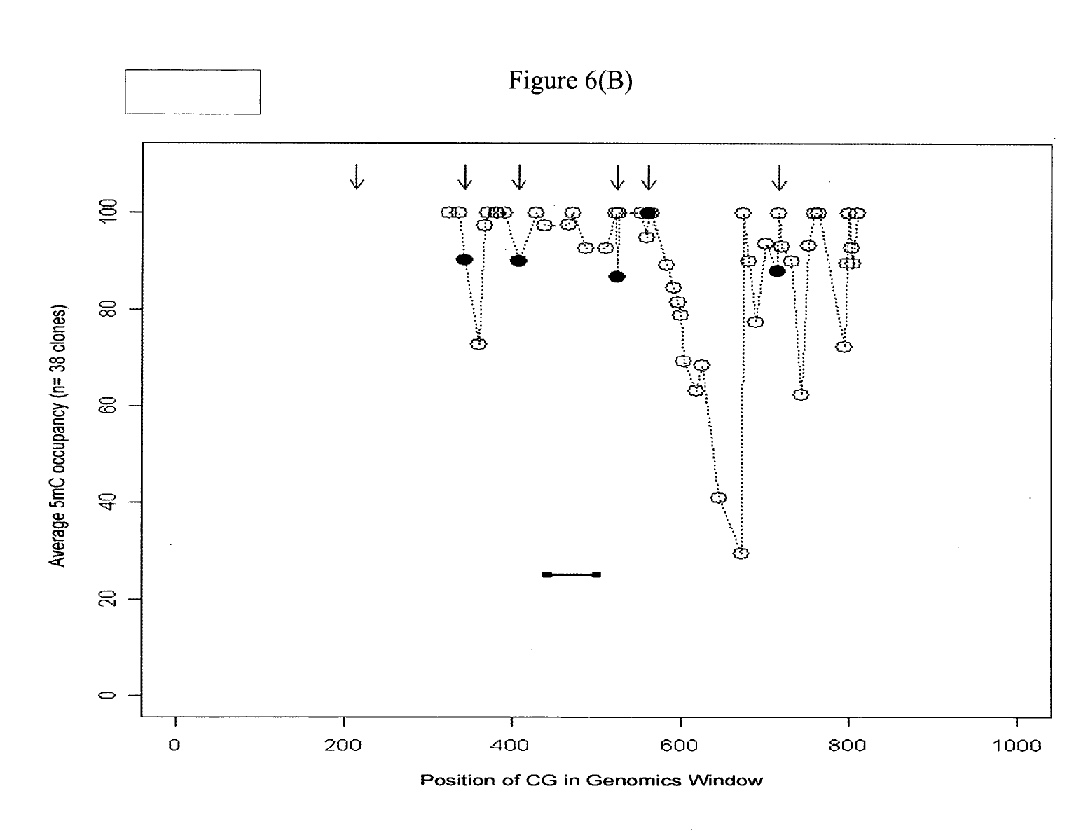

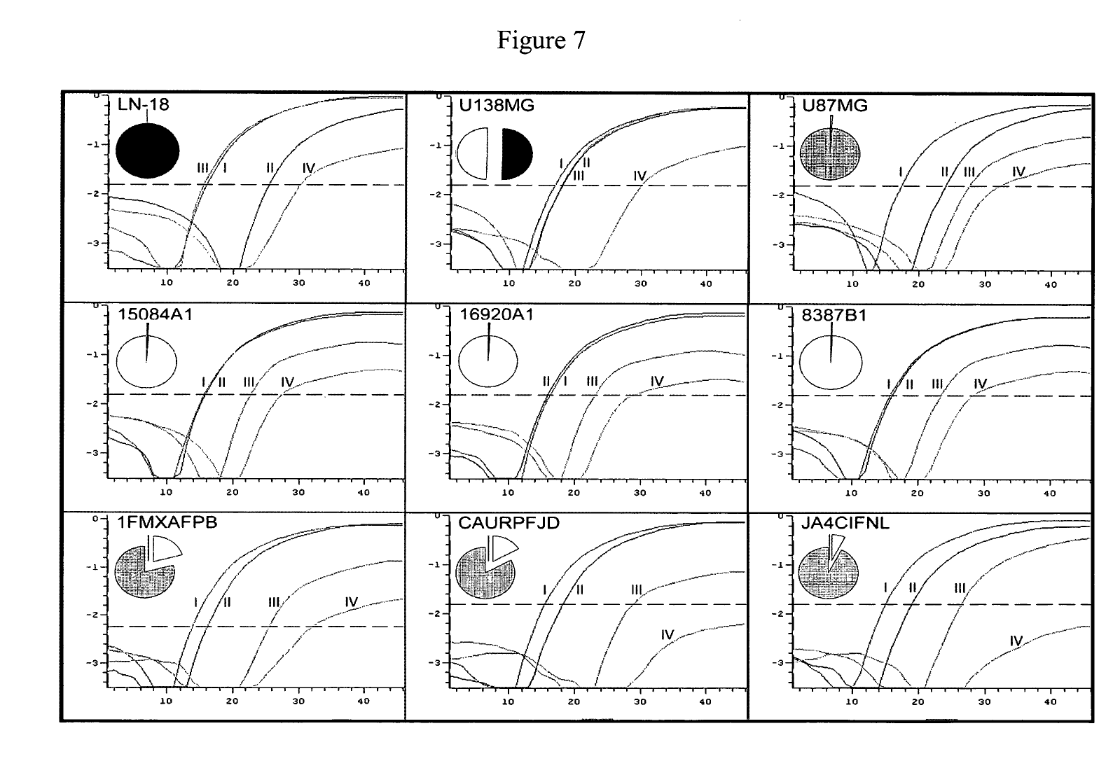

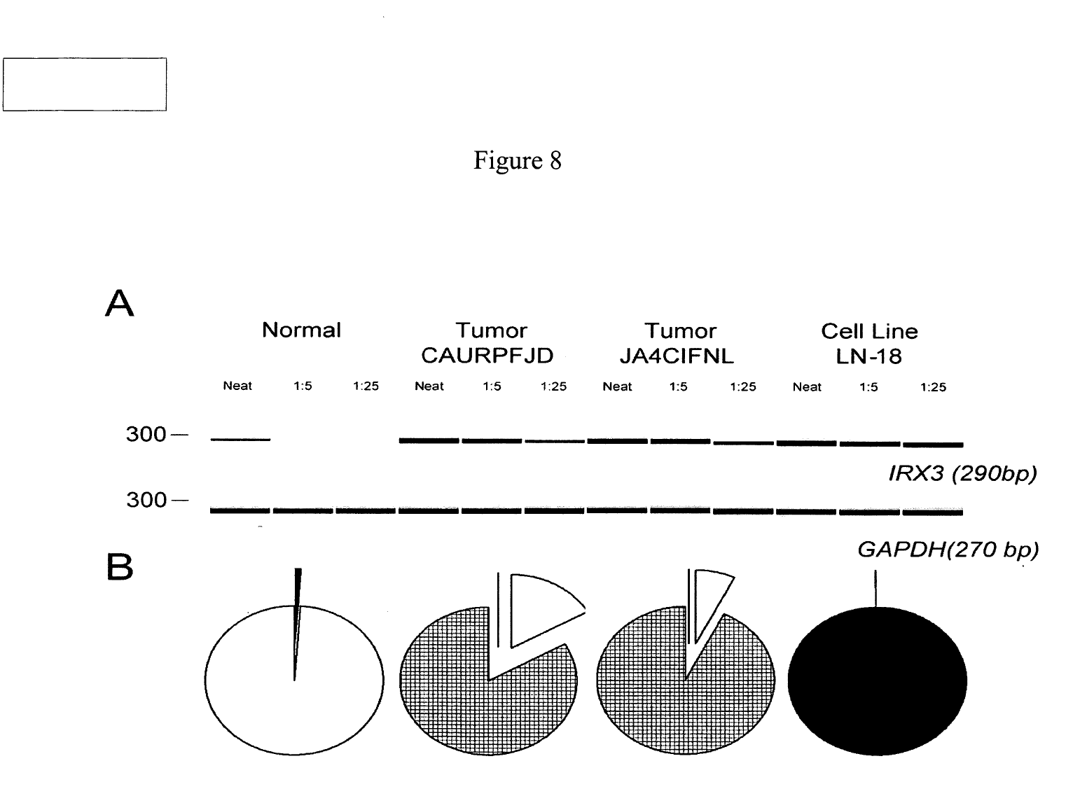

The methylated CGI detected at the IRX3 locus corresponds to an exon rather than the gene's promoter (FIG. 6). A high resolution analysis of the annotation and DNA methylation data obtained from the analysis of the LN-18 genome is depicted in FIG. 6A. The promoter of IRX3 was significantly unmethylated, while the CGI in exon 2 was methylated. Bisulfite genomic sequence was obtained from the IRX3 exonic CGI (FIG. 6B). The graph displays the average methylation occupancy per CG in the interval when considering the 38 clones sequenced, the X and Y axes are the same as those in FIG. 4B. Quantitative PCR and bisulfite sequencing analyses demonstrated that this region is unmethylated in normal blood and normal brain DNA (FIG. 5). The position of the array feature is denoted by the gray bar in FIG. 6B. There are six HhaI restriction sites in the analyzed region, 5 of which appear to be nearly always occupied. The amplicon designed for the qPCR analysis presented in FIG. 5 surveys all six of these HhaI sites. This amplicon was then employed in an assay (FIG. 7). The kinetic profiles obtained from each of the four conditions of the assay obtained from each genomic sample are displayed. The data depict results from cell lines (top row), normal brain tissue (middle row) and three brain tumors (bottom row); each genome's results are color coded by their treatment: mock restriction, HhaI restriction, McrBC restriction, and a HhaI+McrBC double digest. The exon was hypermethylated in all the tumors and cell lines relative to the three independent normal brain samples, reminiscent of the differentially methylated regions (DMR) in imprinted genes like IGF2 and H19. Since the results from normal brain tissues are not consistent with hemizygous methylation, IRX3 is not imprinted (FIG. 7, middle row).

Because the data obtained form the exonic CpGI at IRX3 appeared to consist of five distinct molecular phenotypes we confirmed the results with pursue bisulfite sequencing. The archetypes for each class were LN18, U87MG, U1368MG, Normal brain sample 15084A1 and tumor sample JA4CIFNL. 2 μg of genomic DNA from each was bisulfite converted with an EZ DNA Methylation Kit (Zymo Research) using the manufacturer's recommended protocol. Primers for the bisulfite converted DNA were:

5′ GAGGGATTAGATTTTGGGTTTTTGTAG 3′
5′ AATAACCCTCACCCAAATATCCACCTAATT 3′

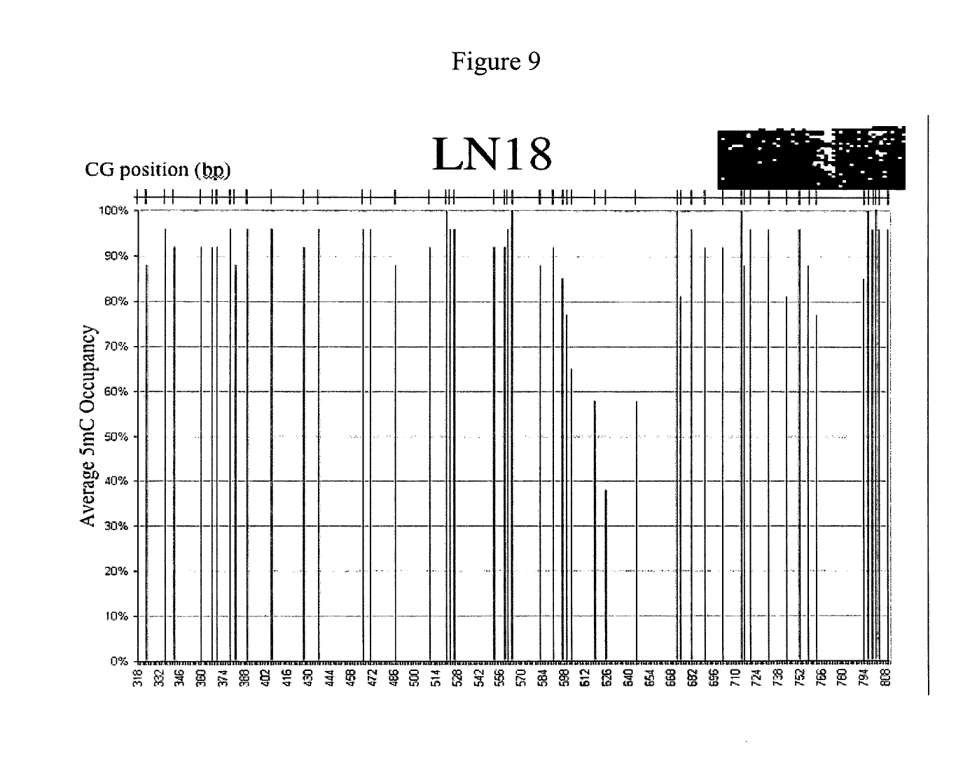

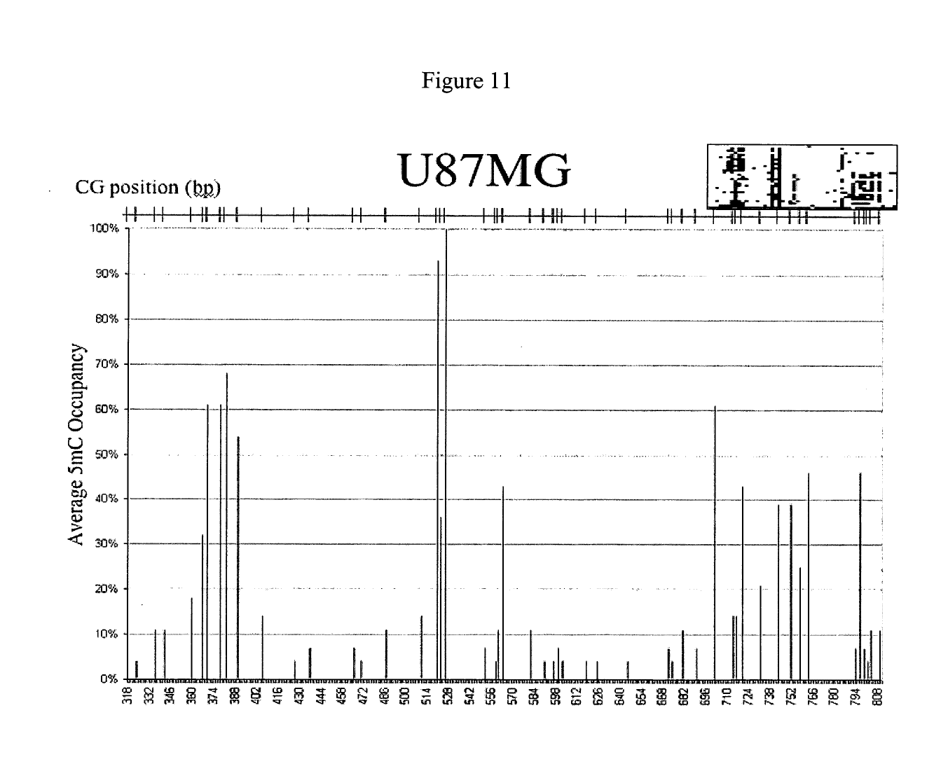

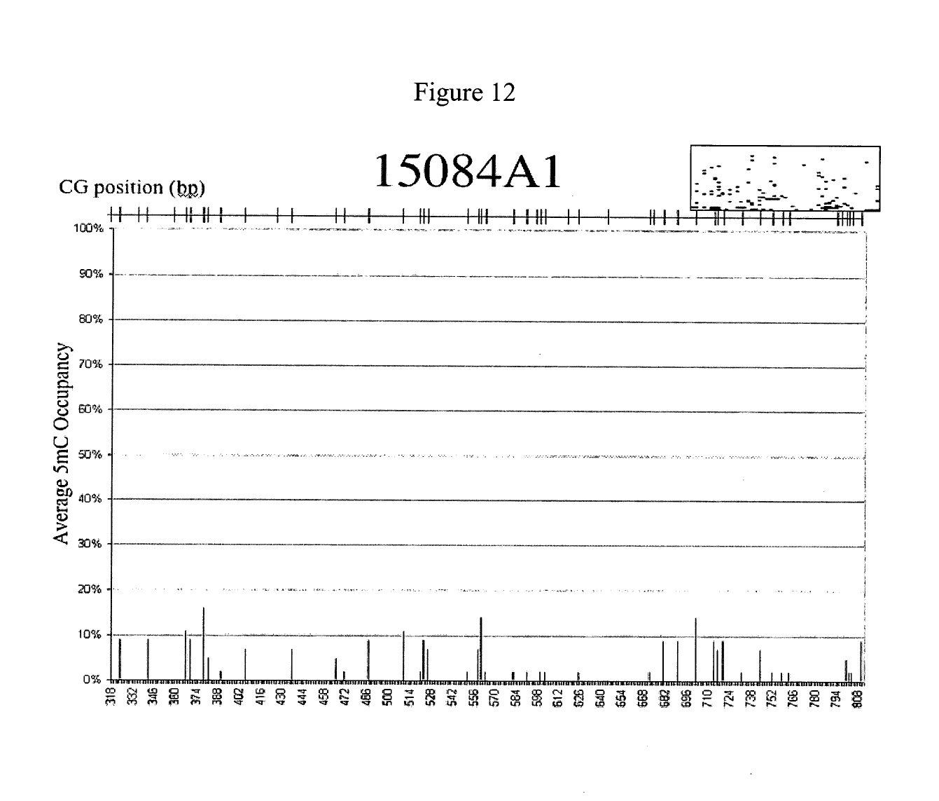

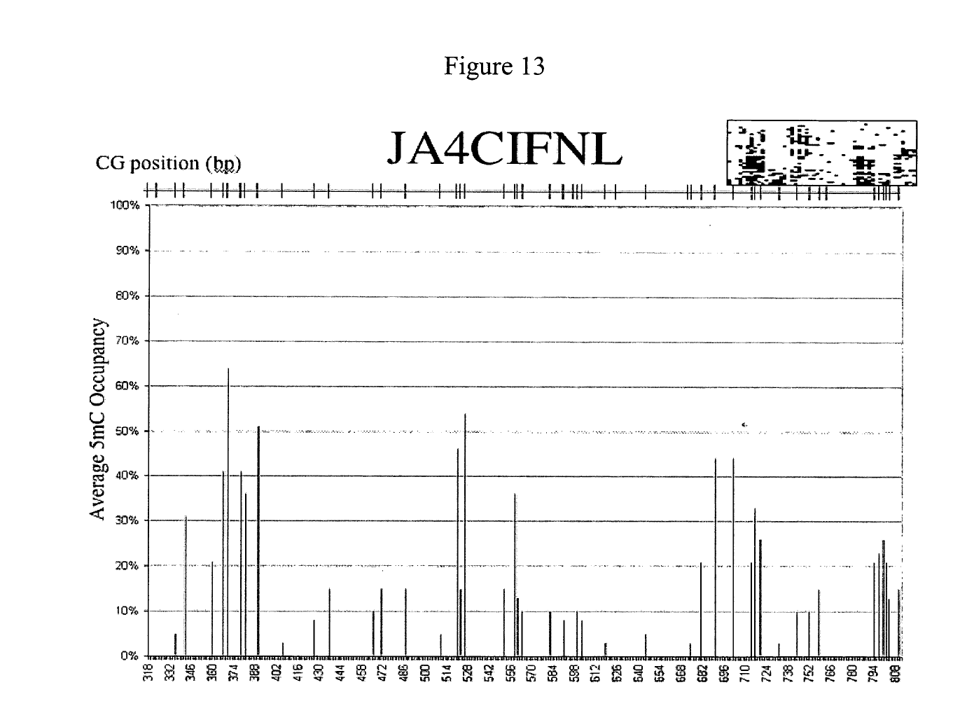

Standard PCR conditions were utilized for the amplification. The PCR products were gel purified and then TA cloned generating a library for each sample. More than 25 clones were analyzed for each of the libraries. The clones were sequenced and analyzed using MethylMapper (Ordway et al, 2005, BioTechniques). The results from the analysis are depicted in two representations in FIGS. 9-13. The predictions were very accurate. The tumors did have the exon hypermethylated and the pattern was most similar to that observed from U87MG.

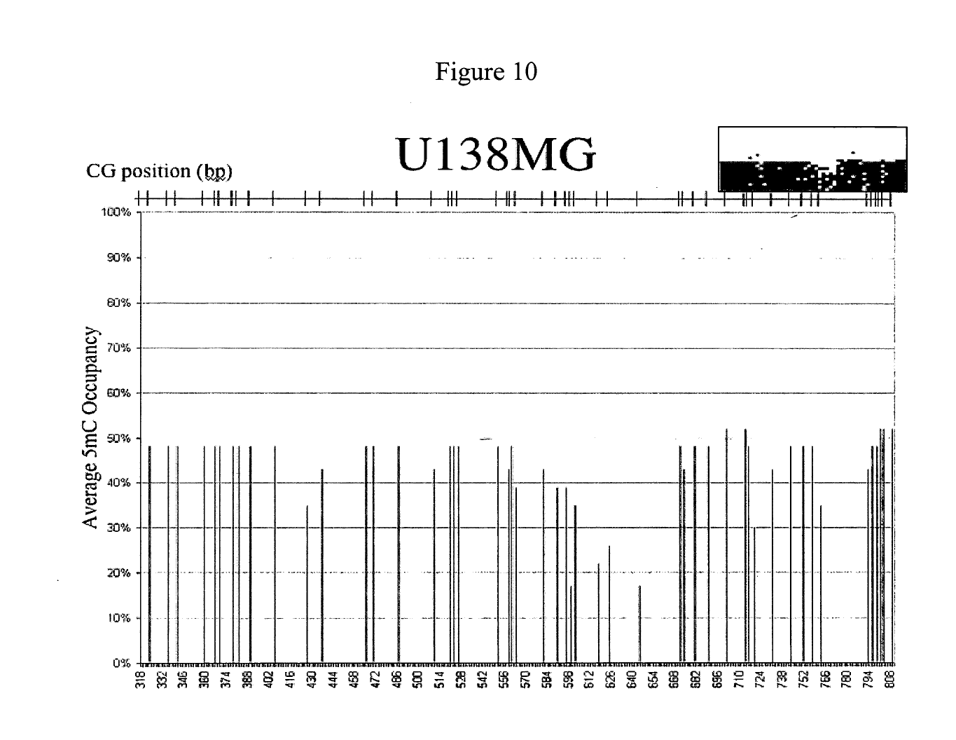

IRX3 is over-expressed (due to its intragenic position) rather than silenced. The expression of IRX3 in tumor and normal brain samples was assessed using semi-quantitative PCR amplification of dilutions of oligo-dT primed cDNA libraries. IRX3 expression was measured relative to GAPDH expression as a positive control (FIG. 10A). The IRX3 DNA methylation result from each sample is depicted in FIG. 10 B. The expression amplicon selected for each target spanned an intron of ˜100 bp or more to eliminate genomic DNA contamination of the cDNA libraries. In all cases, the level of DNA contamination was negligible. Comparison of amplification from each dilution indicated that all three of the tumors expressed between 5 and 8 fold more IRX3 than the normal tissue library (with a SD of 3 fold). Commercially acquired astrocytoma cDNA libraries behaved similarly, although matched DNA samples were not available. LN-18 produced more than 20 fold more IRX3 than normal brain, and had the most methylated DNA (FIGS. 10A/B and 9). The U87MG cell line expressed ˜4 fold less IRX3 than LN-18, but ˜5 fold more than the normal tissue, making its expression level most similar to the tumor libraries. It was also the sample with a methylation pattern most similar to the tumors (FIG. 7).

Although the invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. A method for determining the methylation status of an individual, the method comprising:
obtaining a biological sample from an individual; and
determining the methylation status of at least one cytosine within a DNA region in a sample from the individual where the DNA region is selected from the group consisting of SEQ ID NO: 40, 41, 42, 43, 44, 45, 46, 47, 48, and 49.

2. The method of claim 1, wherein the determining step comprises determining the methylation status of at least one cytosine in the DNA region corresponding to a nucleotide in a marker, wherein the marker is selected from the group consisting of SEQ ID NO: 30, 31, 32, 33, 34, 35, 36, 37, 38, and 39.

3. The method of claim 2, wherein the determining step comprises determining the methylation status of the entire marker.

4. The method of claim 1, wherein the sample is from brain tissue or cerebral spinal fluid.

5. The method of claim 1, wherein the methylation status of at least one cytosine is compared to the methylation status of a control locus.

6. The method of claim 5, wherein the control locus is an endogenous control.

7. The method of claim 5, wherein the control locus is an exogenous control.

8. The method of claim 1, wherein the determining step comprises determining the methylation status of at least one cytosine in at least two DNA regions.

9. A method for determining the presence or absence of brain cancer in an individual, the method comprising:
a) determining the methylation status of at least one cytosine within a DNA region in a sample from the individual where the DNA region is selected from the group consisting of SEQ ID NO: 40, 41, 42, 43, 44, 45, 46, 47, 48, and 49;
b) comparing the methylation status of the at least one cytosine to a threshold value for the marker, wherein the threshold value distinguishes between individuals with and without brain cancer, wherein the comparison of the methylation status to the threshold value is predictive of the presence or absence of brain cancer in the individual.

10. The method of claim 9, wherein the determining step comprises determining the methylation status of at least one cytosine in the DNA region corresponding to a nucleotide in a marker, wherein the marker is selected from the group consisting of SEQ ID NO: 30, 31, 32, 33, 34, 35, 36, 37, 38, and 39.

11. The method of claim 10, wherein the determining step comprises determining the methylation status of the entire marker.

12. The method of claim 9, wherein the sample is from the brain or cerebral spinal fluid.

13. The method of claim 9, wherein the methylation status of at least one marker from the list is compared to the methylation value of a control locus.

14. The method of claim 13, wherein the control locus is an endogenous control.

15. The method of claim 13, wherein the control locus is an exogenous control.

16. The method of claim 9, wherein the determining step comprises determining the methylation status of at least one cytosine from at least two DNA regions.

17. A computer-implemented method for determining the presence or absence of brain cancer in an individual, the method comprising:
receiving, at a host computer, a methylation value representing the methylation status of at least one cytosine within a DNA region in a sample from the individual where the DNA region is selected from the group consisting of SEQ ID NO: 40, 41, 42, 43, 44, 45, 46, 47, 48, and 49; and
comparing, in the host computer, the methylation value to a threshold value, wherein the threshold value distinguishes between individuals with and without brain cancer, wherein the comparison of the methylation value to the threshold value is predictive of the presence or absence of brain cancer in the individual.

18. The method of claim 17, wherein the receiving step comprises receiving at least two methylation values, the two methylation values representing the methylation status of at least one cytosine markers from two different DNA regions; and
the comparing step comprises comparing the methylation values to one or more threshold value(s) wherein the threshold value distinguishes between individuals with and without brain cancer, wherein the comparison of the methylation value to the threshold value is predictive of the presence or absence of brain cancer in the individual.

19. A computer program product for determining the presence or absence of brain cancer in an individual, the computer readable product comprising:
a computer readable medium encoded with program code, the program code including:
program code for receiving a methylation value representing the methylation status of at least one cytosine within a DNA region in a sample from the individual where the DNA region is selected from the group consisting of SEQ ID NO: 40, 41, 42, 43, 44, 45, 46, 47, 48, and 49; and
program code for comparing the methylation value to a threshold value, wherein the threshold value distinguishes between individuals with and without brain cancer, wherein the comparison of the methylation value to the threshold value is predictive of the presence or absence of brain cancer in the individual.

20. A kit for determining the methylation status of at least one marker, the kit comprising:
a pair of polynucleotides capable of specifically amplifying from human genomic DNA at least a portion of a DNA region where the DNA region is selected from the group consisting of SEQ ID NO: 40, 41, 42, 43, 44, 45, 46, 47, 48, and 49; and
a methylation-dependent and/or methylation sensitive restriction enzyme and/or sodium bisulfite.

21. The kit of claim 20, wherein the pair of polynucleotides are capable of specifically amplifying from human genomic DNA a marker selected from the group consisting of SEQ ID NOs: 30, 31, 32, 33, 34, 35, 36, 37, 38, and 39.

22. The kit of claim 20, wherein the kit comprises at least two pairs of polynucleotides, wherein each pair is capable of specifically amplifying from human genomic DNA at least a portion of a different DNA region.

23. The kit of claim 20, wherein the kit further comprises a detectably labeled polynucleotide probe that specifically detects the amplified marker in a real time amplification reaction.

24. A kit for determining the methylation status of at least one marker, the kit comprising:
sodium bisulfite, primers and adapters for whole genome amplification, and polynucleotides to quantify the presence of the converted methylated and or the converted unmethylated sequence of at least one cytosine from a DNA region that is selected from the group consisting of SEQ ID NO: 40, 41, 42, 43, 44, 45, 46, 47, 48, and 49.

25. A kit for determining the methylation status of at least one marker, the kit comprising:
a methylation sensing restriction enzymes, primers and adapters for whole genome amplification, and polynucleotides to quantify the number of copies of at least a portion of a DNA region where the DNA region is selected from the group consisting of SEQ ID NO: 40, 41, 42, 43, 44, 45, 46, 47, 48, and 49.

26. A kit for determining the methylation status of at least one marker, the kit comprising:
a methylation sensing binding moiety and polynucleotides to quantify the number of copies of at least a portion of a DNA region where the DNA region is selected from the group consisting of SEQ ID NO: 40, 41, 42, 43, 44, 45, 46, 47, 48, and 49.

27. A method for determining the presence or absence of brain cancer in an individual, the method comprising:
a) measuring the amount of IRX3 RNA or IRX3 protein in a sample from the individual;
b) comparing the amount to a threshold value for IRX3 RNA or IRX3 protein, wherein the threshold value distinguishes between individuals with and without brain cancer, wherein if the amount of IRX3 RNA is measured, the amount is compared to a threshold value for IRX3 RNA, and if the amount of IRX3 protein is measured, the amount is compared to a threshold value for IRX3 protein, and wherein the comparison of the amount to the threshold value is predictive of the presence or absence of brain cancer in the individual.

28. The method of claim 27, wherein the sample is from the brain or cerebral spinal fluid.


SEQUENCE LISTING (51 sequences)

SEQ ID NO: 1 - DNA, 60 bp, Artificial Sequence
  E-cadherin (ECDH1) ha1c_00072 feature
gattcgaacccagtggaatcagaaccgtgcaggtcccataacccacctagaccctagcaa

SEQ ID NO: 2 - DNA, 60 bp, Artificial Sequence
  RASSF1A ha1c_00077 feature
cttcctctagcacagtaaagctggcctccagaaacacgggtatctccgcgtggtgctttg

SEQ ID NO: 3 - DNA, 60 bp, Artificial Sequence
  HOXA10 ha1p_10831 feature
tggatactggaagtagaggcaagtcagcttctagttcactctgggctcctgacactctga

SEQ ID NO: 4 - DNA, 60 bp, Artificial Sequence
  ZGPAT ha1p_33140 feature
ttgttctgttcccatcaacaagtctgagtctagcagcccagcatttcccaccaactttga

SEQ ID NO: 5 - DNA, 60 bp, Artificial Sequence
  BMP7 ha1p_57533 feature
gaaacaacctcagcttggcaggtcttggaggtctctgggaggagaaagcagcgtctgatg

SEQ ID NO: 6 - DNA, 60 bp, Artificial Sequence
  IRX3 ha1p_58072 feature
ctgggacccatttcggactccaaaaatagcgactcggaagatagctctgagggcttagag

SEQ ID NO: 7 - DNA, 60 bp, Artificial Sequence
  WNT6 ha1p_72837 feature
atttttgtggcattagaaaatctgataaacgccatctcacctgcagctggctctcccacc

SEQ ID NO: 8 - DNA, 60 bp, Artificial Sequence
  WNT10A ha1p_72985 feature
gcgttcggttctcgctcttccgctaatttgctgtgtgacctctttgagcctcgatatttt

SEQ ID NO: 9 - DNA, 60 bp, Artificial Sequence
  RARAlpha ha1p_94854 feature
gatgaccagacgcccatctttcttccagtgggttacttctcccccatcagtccttcgctc

SEQ ID NO: 10 - DNA, 23 bp, Artificial Sequence
  testis specific histone 2B (TH2B) L primer
tttgctatctgattggctgatgg

SEQ ID NO: 11 - DNA, 26 bp, Artificial Sequence
  E-cadherin (ECDH1) L primer
agaccccatctccaaaacgaacaaac

SEQ ID NO: 12 - DNA, 25 bp, Artificial Sequence
  RASSF1A L primer
cttgcccttccttccctccttcgtc

SEQ ID NO: 13 - DNA, 27 bp, Artificial Sequence
  HOXA10 L primer
tctggaactggccccttgtctgaattc

SEQ ID NO: 14 - DNA, 28 bp, Artificial Sequence
  ZGPAT L primer
gccctttctttacctccactcctcattc

SEQ ID NO: 15 - DNA, 23 bp, Artificial Sequence
  BMP7 L primer
cggaggtggcgcggaaatagaag

SEQ ID NO: 16 - DNA, 19 bp, Artificial Sequence
  IRX3 L primer
cgctgcccccggtggagac

SEQ ID NO: 17 - DNA, 25 bp, Artificial Sequence
  WNT6 L primer
ttgatggtatggcagggagaatgtc

SEQ ID NO: 18 - DNA, 23 bp, Artificial Sequence
  WNT10A L primer
gggcacaggcaggcaacactcag

SEQ ID NO: 19 - DNA, 21 bp, Artificial Sequence
  RARAlpha L primer
cccacctccagcaccaaaacc

SEQ ID NO: 20 - DNA, 23 bp, Artificial Sequence
  testis specific histone 2B (TH2B) R primer
aagcccttcttggaaatggtagc

SEQ ID NO: 21 - DNA, 20 bp, Artificial Sequence
  E-cadherin (ECDH1) R primer
ccacccggcctcgcatagac

SEQ ID NO: 22 - DNA, 24 bp, Artificial Sequence
  RASSF1A R primer
agagcgggcggtaaagctgctgac

SEQ ID NO: 23 - DNA, 23 bp, Artificial Sequence
  HOXA10 R primer
agtcccgccagcccaccaaagtc

SEQ ID NO: 24 - DNA, 26 bp, Artificial Sequence
  ZGPAT R primer
ctcgcctttctggttttctggtcttc

SEQ ID NO: 25 - DNA, 23 bp, Artificial Sequence
  BMP7 R primer
gaggggcgaggcgaggaaacaac

SEQ ID NO: 26 - DNA, 23 bp, Artificial Sequence
  IRX3 R primer
gcgcgaggaggaagacgaagagg

SEQ ID NO: 27 - DNA, 22 bp, Artificial Sequence
  WNT6 R primer
ccgggtagaaggggtgggagag

SEQ ID NO: 28 - DNA, 24 bp, Artificial Sequence
  WNT10A R primer
gcccccagaaaatgcttgcgtagg

SEQ ID NO: 29 - DNA, 20 bp, Artificial Sequence
  RARAlpha R primer
tccccgccctcttccaccag

SEQ ID NO: 30 - DNA, 344 bp, Artificial Sequence
  testis specific histone 2B (TH2B) Amplicon
tttgctatctgattggctgatggccgtctacccaatcagaaagtcgtactagaatcgcctcatttgcattcacgccacttcccattgtccaatcagattttggataccgaacgcggcgtttgagggccgtgcctataaataccgcatctttcatcctccagttctgtttgtttacttggcgagacttggagctgaggtcatttggagctgtttaatactgaagagctgttgagcactggaaagtgctgtgtaaccctggaaaagaaccgtgtaacgctgcagaagtgtgtggtagctatgccggaggtgtcatctaaaggtgctaccatttccaagaagggctt

SEQ ID NO: 31 - DNA, 213 bp, Artificial Sequence
  E-cadherin (ECDH1) Amplicon
agaccccatctccaaaacgaacaaacaaaaaatcccaaaaaacaaaagaactcagccaagtgtaaaagccctttctgatcccaggtcttagtgagccaccggcggggctgggattcgaacccagtggaatcagaaccgtgcaggtcccataacccacctagaccctagcaactccaggctagagggtcaccgcgtctatgcgaggccgggtgg

SEQ ID NO: 32 - DNA, 275 bp, Artificial Sequence
  RASSF1A Amplicon
cttgcccttccttccctccttcgtcccctcctcacaccccaccccggacggccacaacgacggcgaccgcaaagcaccacgcggagatacccgtgtttctggaggccagctttactgtgctagaggaagagggtccccacatccggccctggccctcctggtccggtttgctgaagcaacacacttggcctacccactgggtggggcaggaagtctcgagccttcacttggggtgaggaggagggagatcggtcagcagctttaccgcccgctct

SEQ ID NO: 33 - DNA, 298 bp, Artificial Sequence
  HOXA10 Amplicon
tctggaactggccccttgtctgaattctgcctcctcaaaagtggcgaacctggccctatggccgtcaggatcctcagagtgtcaggagcccagagtgaactagaagctgacttgcctctacttccagtatccacagatttttccccaaaatgcagtggttgttccctagcccctaacccccaacacttttctcccctagtccgttgatccagttaggatcttctcctctggtgttgtgctcacccggcttgccttctctacaactcctgtcagtggactttggtgggctggcgggact

SEQ ID NO: 34 - DNA, 290 bp, Artificial Sequence
  ZGPAT Amplicon
gccctttctttacctccactcctcattccattccactcaaagttggtgggaaatgctgggctgctagactcagacttgttgatgggaacagaacaattaattttttttccgaatttatatttcccggcacaagcacaaatgctcagccaggtcccttcaggcaccgggaaatcatcccggatacccaagccgacttttgagcaagcacagcccatggaaagggcagtcccgccggccagccccaagcgagaatctagttggtgagaagaccagaaaaccagaaaggcgag

SEQ ID NO: 35 - DNA, 265 bp, Artificial Sequence
  BMP7 Amplicon
cggaggtggcgcggaaatagaaggcttcctggaagcggtgacccttgagccggtcttgcccctcagtccctgtatccttttaagccagccacgctgacccctccgcgcctccctgaagccgttgccagcctgtctctgcgctcgccattccctccgcctggacctctccccactcaccacctcccgcccccatcagacgctgctttctcctcccagagacctccaagacctgccaagctgaggttgtttcctcgcctcgcccctc

SEQ ID NO: 36 - DNA, 505 bp, Artificial Sequence
  IRX3 Amplicon
cgctgcccccggtggagacccccccgcgccgggaggcgagcggcgcgggttgtccgggcttgtggcagtctccgcgagggaccagatcttgggcttctgcagggcggaggcgggggctggtgcgggagcgcaggggtccaggctcacggggggcgacggcagagacggcgaggccacggccactggtggtggcgctggagccagactcaggaccggtagtggccggtcctctaagccctcagagctatcttccgagtcgctatttttggagtccgaaatgggtcccaggcctaggtcgccatccctgcgcgccgccccagccagggacagctcaggctcggtggccgcgccgtctaagttctccaaatcgatctcctcgtcctcgtcgtcgtcagccaggccctcgccccccgtgtcctcctcctcccccccgagctcctcctcctccagctctagctcgcgtttgccgtcctcctcgtcctcctcttcgtcttcctcctcgcgc

SEQ ID NO: 37 - DNA, 259 bp, Artificial Sequence
  WNT6 Amplicon
ttgatggtatggcagggagaatgtcccacaccacctgtcccttggcaccccggggcacgtcggggacgggaggggcaggggctgctgcccactgccagccgaatggcatgatgaatggagctgtccctcttccccacggagcctttgatctccccctcgtggggagggggccaggggcatcaaagacatttttgtggcattagaaaatctgataaacgccatctcacctgcagctggctctcccaccccttctacccgg

SEQ ID NO: 38 - DNA, 248 bp, Artificial Sequence
  WNT10A Amplicon
gggcacaggcaggcaacactcagtgttggggaggcagctgctgcgctgggagtcaggagagctgcgttcggttctcgctcttccgctaatttgctgtgtgacctctttgagcctcgatattttcttgtgaaataaaggggagctaaataaactatctcaacggccacttctagggttaaaagtctctctaacgcctcctcccaggacgtgccaccccgggcgctcctacgcaagcattttctgggggc

SEQ ID NO: 39 - DNA, 250 bp, Artificial Sequence
  RARAlpha Amplicon
cccacctccagcaccaaaacctaaatccatctgctcagggcaggcaggaagaaatgtctattttctcctgtttcaggtcatcctatcctcgagttatccatatgctgtgtatttgagacagggaactgttcctgtccccagccgatgaccagacgcccatctttcttccagtgggttacttctcccccatcagtccttcgctctgcgcccgctgacccagtcggggctggctggtggaagagggcgggga

SEQ ID NO: 40 - DNA, 8000 bp, Homo sapiens
  testis specific histone 2B (TH2B) DNA region
atggcaaaacgctgactctactaacaatacaaaaaaaaaaaaaaaattcgctgggtgtggtggcaggcgcctgtaatcccagctactcaggaggctgaggcaggagaatcgcttgaagccgggaggcggaggttgcagtgagctgagatggtgccattgcactccagcctgggtgataagaacaaaactccgtccctgccctccccacccaaaaaaaaaaaaaagtaaagggaaaaaagtggcatatatttgtctcagttgctgctgaagttttattgttaatagccaatatttatttttaattaacaacttttagcaattttggaattacacaaatcccataaaaacccagcattaggcataagacagtgtcatattagtctgattataataaaaaataagtcagccgggcatggttgctcacgtctgtaatcccagcactttaggataaggccaaggtgggtggtcaggagttcgagaccagcctggccaacatggtgaaaccccgtctgtgctaaagatataaacattagccagacatgatggtacatgcctgtaatcccagctacttgggagaatgagggaggagaatcgcttgaatccaggaggcagaggctgcagtgagctgagatcaggccactgcactcgtctgtgcgacagaggaaggctgttgggggggaaaataagtcaatatcaccatttttattatcacgtgtaactattagacaatgcatttagacaaagtcaactatttaaaacaacaggacaaaattatatgatttttctgcctacctggaaaaccacagaggaaaaaagctggacaacagaaatgacagaataaggaaggaaatacaaaccagtacgaatatcaagaatgttctatctgtaaaacatcgtcaatcagaaaacataataaaagaaacccttctaatatttccccaaaatacagtaataaaaaagaatacctggagataaactctatgaaacgtgtgaaagttctataagaagaaatcaaggctgggtgcggtggctaacacttgtaatcccagcactttgggaggctgaggctggcggatcacctgaggtcaggagttggagaccagcctggccaacatggcgaaaccctgtctctactaacaatacaaaagttagctgggcgtggggcaggcgcccgtaatcccagctactcaggaggctgaggtaggagaatcacttgaacctgggagctggaggttgtaatgagccgagatcacgccactgcactccagcctgggtgacagagcgagactctggctccaaaaaaagaagaatcagaaacttctgtaggtaaattgttttgaaaaatcaagggggcatgtctcctgtaacatgtattcacaattttttaatatattttattctatctaaacatttagtttaatgcaattttaataggattgcattagagatagaaataaaagtattatgtagtgcatttggaataaaagaatacacaggttctcttctataaagtcaagtccctcctcctctgccttggcaagtctgagaagttaagtgccactttcttgacaaagtatctacattaattacttggaattcatctgtttaggtttgtcttatatcctgattttaagttttacatttgccatgttctaaatattacccagttttattcaacatatagttgccatttctgttttcaaatatcttcaatttttgccgtccctaactgcatcccttatttattatgtatgcaggttaattcatacttttgtaacttgattgtccttactcggcatgttttattttatagaactttacctgcttctatacatgccttattatgtgtagacatcaagtgtttagcagttttttttttggtaaccaaatgattttttttttttttgaggtaaggtcaactgttgaatatcgtacaataatttttaatttagttatttggcagcgttactgaatgggttttgcttttcactttctcttggtgtttacagcacaaactgcaaacacagatcaacaaactattttgcctcttttcaacagtcaaaacttttgaagcatatactcttccaaatttctaaacttcaaattatgttcggccttctagtcccattcctgcctgagtctaacactagtatctcaccatcacatatggcgtaaggcttctttttgacatattttacttacattttaagaaataattctattattctttacaaagtacgtgaaaccacaggtatcaaatatatatgcttacccaaattttaagattaacttacttacatgccacatttaattatgagtatttcgtgaaactaccatatttgaaaggaaattctctaaaatgtactttaaccagccagactgatgttacctggatttaaacaggaagattacacctctcctgcttgtaatgcaattttcacttccatgtgtaacccttaacataactaacatacataaatctagtttgagatagtctcgctcatgtcacccagactggagtgcagtggagcaatcaatcttcgttcactgtaactttcgcctcccggactcatgaacaaccctcccaccgcagcctcccgaacagctgggactacaggcctgcatcaccagacccagctagtttctgtggggacagggtctcaattatgttaacggtcaagccccaggcttgaacgaccctcccgctccaacctccactgaagtgcaagtactgactgctcacaagcgttaagccacctcgcccagtgacatatataaccaaaattacctcaaactaatggccgatggaacgctccttgaaaattcagtccccgtacattcccgaaatgcgcttaaacgggcatttgtgacaggcaagtaagatggctggttaacagcagtaacgttttgtaacgtacagcctttttctgagacggtaggtggctctgaaaagagccttttgttttttctgacacagaagtctttttaccaatgacaaccttaagttacttgctttgggctttatggtggtgactctcagtcttcttgggcagcagcactgcctgaatgttaggcaggactccgccctgggcaatggtcacgccgcccaaaagcttattgagttcctcatcattgcggatcgctagctgcaggtggcggggaataatgcgagtttttttgttatcgcgagacgcattgcctgccagctcaaggatttctgctgtgagatactctaacactgccgccaaatacactggtgcgcctgcccctatccgctctgcatagtttcccttacgaagcagacgatggatccggcctacgggaaactgcaaacccgctctagaagagcgagacttagacttggcgcgtgcttttcctccctgcttccctcgtccagacatctcctcgcatcaaattgcagcaacacgagaaccacatttctagggctgctactgggcctatttatagtctgactgaggttggcatttgctatctgattggctgatggccgtctacccaatcagaaagtcgtactagaatcgcctcatttgcattcacgccacttcccattgtccaatcagattttggataccgaacgcggcgtttgagggccgtgcctataaataccgcatctttcatcctccagttctgtttgtttacttggcgagacttggagctgaggtcatttggagctgtttaatactgaagagctgttgagcactggaaagtgctgtgtaaccctggaaaagaaccgtgtaacgctgcagaagtgtgtggtagctatgccggaggtgtcatctaaaggtgctaccatttccaagaagggctttaagaaagctgtcgttaagacccagaaaaaggaaggcaaaaagcgcaagaggacccgtaaggagagttattctatttacatctacaaagtgctaaagcaggtccatccggacactggcatctcttcgaaagctatgagcattatgaattccttcgtcactgatatctttgagcgtatagcgagcgaggcatcacgtttggctcactacagcaagcgctccaccatttcttccagagagattcagacagcagtgcgcttgctactgccgggagagctggctaaacatgctgtgtctgagggcaccaaggctgtcactaagtacaccagctccaagtaagcctgctaagtaaacgtcatttctaacccaaaggctcttttcagagccacttaaacatactgaaacagctgtgggcttcgtttttgtgtagtttctaaccgtaagggttttttttttttttgaggcggagtctcactctcccaggctggagtgcagtggcggaatctcggctcactgcaagctctgcctcctgagttcacgccattctcctgcctcagcctcctgagcagctgggactacaggctcccaccactacacccggctaatttttgtatttttagtagagacggggtttcaccatgttggccaggatggtctcaaactcgatctggtgatcacccacctcggtctcccaaagtgctgggactacaggcgtgagccactgcgaatatatccaaatcagctgtgggccgagggtagacgaactagttgcagcaagacaatccttgctaggcaccgtgactcgcctgtaatcccagcacttgcagaggtcgaggctggcggttcatgaggttaagagacggagaccatcttggccaacaaggtgaaactccgtctctactaaaaatacaaaaatcaggtgggcgtggtggcgtgcactgtagtcccagctaattgggaggctgaggcaagagaatcgcttgaacccaggagccggagattgcagtgagccgagatcgtcactgcactacagcctggtgacagcgagactgtctcaaaaaacaaaacaaaaaaaccccctagtttgtataaaagctacatgctgatactggtatatttaaggttagcattacacataataaaactaaaaagcgcagcgacatagacagtataacttgcattatgatcccaaattttaagacctcacaaaaagggaatagtttagaggaagttacgttttgtgccacaggtaagtcaaggtgtggaaatgattgtgcgaactctggagaactcacaaatctggttagcaggaattaaacggatttaaaatgtcccttaattttcttaaatttataatagtttccttcattgtcatgttactggttgttggactccatctgtaagagctcccaagcattaaactgatttcagccaccttaaaaatggatcaaatattacttcctggaaacctgtggttagctggaattctcaatttgtttaacgcatcttcatttaacctttatcttttaacaaaattttggatatgtatatgtgctttctctacattaaaaagttagtaccggccggcgcggtggctcacgcatgtaatcctgcactttgggaggcagaggagggcagatcacgaggtcaggagatcgagaccatcctggctaacatggtgaaaccctgtctctactcaaaatacaaaaaattagctgggcgtggtggcacgttcctgtaattgcagctacttgggaggctgaggcaggagaatcccttgaacccgggaggcggaggttgcagtgagccgagatcgccccactgcactccagcctgggtgacagagcgagactccgtctcaaacacaaacaaaaaaatatatcaatgtataacaagacccgttaaaataagtgcccccacacttttgtgaacagtaagacagtccctgcaaagcccctgcatgctttagctgggaaatcttccagggaggtgacaaattggggggaaggggcatctagcatctatgctcaccttgtattggggatgggatcatctctaatcagggattcatgcttctctaacaccttggaaaacagcaggtatcacatagctgccaaataacctgtcgactttgtgaccactgtcagactaccctgattacccaaggacttttaggcttatttgggcaatagataagtaggaataccagcaaataaaaactgttttcagaccaaacttcatgtagcttttatatcagatgtattttggctgcaaataataggaaatgctagctgaactggttgagataagtaatgcaaccaaacaggacaacataaaggcagagaagttccaccaataggataatcacagccctggctccactaccctgcagtcttgcctctgcccttctccacactgtgactttgccatcatactagttacagttgcaagtgttcatgcagagaggataggataaagtaaaggcaggaagagtcagtctattccactgggtcttggattgaaagcaagaggagtcttcccagaggacccgttcctctcaggtccattttctagaactgagctagttcaggtgctgcctgagtcattcacctacagaaaaccacgggatgggtgtgaactgatcatctaccttcatatatatttggcattttaaaaagttaaatatagttctgggtattttgtttttgttgttactgtaaatgaggtatttcctattcattattttttaactttttttgttatatatgtaagactgctttccgtgcatttttttttaatatccttctagtacctcactgaattgacttattttcctattggtttcttgatagattttcttgagttttacatgtttgcaatataggttgagcatccgtaatccaaaaagctccaacattcaaaacttttattgtactattgatttctttgcaagtctagctgatcctatacacagcttaatataaatttctaagtgcatgcaataaaatacttaatactattaataaaacaataccaaattgagttttcaaaatattttttaaaatgtgtgatatagtaatatttgtgcttcaaataagattgaaaatggtaatattttagtgcatacattgtggaaaacagtatagaatttcctcaaaaaattaaaaatagaactaccatataatccaacaatcctactactgggtatatattgaaaggaaataaaatcagtttctcaaagagatatctgtactcccatgttcattgcagaattattctcaatagacaagatatagaaagaacataaaggtccttcaggagatgaatggctaaagaaaatgtggcatacatacataatagaatactatttggcctcaaaaaagaaaaagaattctgtcattggcagcaacatggatgaacctggaggatgttaagtgaaatgagcgaggcacagaaagacaaatgctgtatgatctcataagtggaattaaaaaaaagttgaactcatataagtagcagagtggtggttaccagtggctagagttggggagtggtgggaagatattggttaaaggatatgaaatttcacttaggaggaataagctgaagagctctattgtacaatatgctgattatagttggtaacaatatgttgtattgaaaattgcaaagagtagagtttaagtgttttcataaaaaatatgtggggtaatgcatatgttaattagattgccattccgcgatgtatacatatttcaaaacatcatgttgtacacagtaaatacacaatttttgtcaatttaaaaaatgaaaatagttgggtggagagtggtttttagggaggcaagattaagagcagtgagattaattggaaactaattcaggtgtgagatcatgagtatgcagacaaaaatgttagcggtggagatggtgtataatcagacaacagaaatatgtaggaagtaaaattaccatatcttgagagactgaagtggatggaggttgaacagagactattagggaaaaaatacatttctggcttggacaaccatgtgaatagggtggtactcatggatatagagaatgcagaagttcaggaacagttctgttgggatcaactggaaatccctgtggagataattcttaagcggctaagcaacataagaactgtga

SEQ ID NO: 41 - DNA, 8000 bp, Homo sapiens
  E-cadherin (ECDH1) DNA region
atgaattaagtgctatcaacattcccattttacagatgaggaaaccaagatggtggagataaaatgacttatgcttctcaactaatggcagagccagaagtgaatccaggctggagtgaagtggctccagtgctcaaatgatcctcctacctcagcctccagagtagctgggactacaggcatgcactgccatgtccagctaattcaaaataatatttgttggggagatggggtttcaccatgttgcccagactggtctcaaactcctgggctcaagtaatcttctcacctcccaactagcctcccaaagtgctgcgattacagttgtgagccaccaggcctgcttaggtatccctgtttgatacaagagaaaacagagggcaggcacagtggctcacacctgtaatcccagcactttgggaggctgaggtaggtggatcacttgaggtcaggagtttgagaccagcctggccaacatggcgaaatcccgtctctactaaaatacaaaaattagccagacatggtgacgtgtgtctgtaatcacagctactcaggaggctgaggcacaagaatctcttgaacctgggaggcagagagtgcagtgagccgagatcgcgccattgcactccagccggggcaacagagcaagaccttgtcactaaataaataaataggaaacagagaagcaattcagtgtgttgcgaaagtcacacagcacctgtcttccacaaggagcccctctgtgttcccgcctcaccctggacacctgatctactgtctttggagcttggagaaggacttgccgagaagtctgaagaatgcatcaggcctgacctgactttgctgcccctaattgttttctaaaatcctttttattgaattatcagagtgtaatggaagagtcgatgagagtcgctgcagagttacgaggccaaggcttgctgggtagaagaaagtaaagccctaaaggctcccagggcccagcgatggtataaaatatgtatttttacataactgagaggaaaaaaataagcagacagtacagccccatgggggaaacgcctctttctgtatttccattatcttgttcaaggcacgatcctagtgggaaaggaaataccggagccttctccctctgagcctcctggtgggttaagagcagagggagccaaaatacgacatccaaaagacctgattggaaactcattaaaattgaggggacaggttgttggggggggcggtgctgggctgcccagaaatcgtctgcatttgagaaattacttgatggactcagaggcacctttagctctgggtgaaatattccaaacatggctttccccaggaggaagcgaaataggggctgggccttttgaaagagtaacccctactccaaatatttattgagcagttgctatgatgagccaagaactctgcttagcgattgtttttgtattagctcatgccaattcacttatacaaaggagaacactgacttccccaaggtcacagggctagtgagtggctgactccacttaatgctgtgtagttctaaccatgcttgaaggataaagggaaaggaggtgcccttgtcggggtagacaagccacccagacgtggctggccacagggagtcagacagggaagttgtctaacctaacatttgcctgtctattctgtttttgtcttttgatggaggacggcgggtgctggagggccactggcccaggacctgagacctttggcccctatctattctatttttgatcctaaaattatttcactggggacagaattctctggagaaggtgatggtggccatggtgtggctgtggcattaaaagccccactctgggggtcagaagatcctgggttttggtctttggctttgacattgacctgcctatgtgacattgaacaagttatttgcaaatcataggtttgggtgaactctaaaggtctcttccaggtcaacatcaaaaaacagactgaaaactgcagctccagctggaagactgggaccacatgagatgaagctgattgttagaagaaaggccctggaatcagagaggttggttccagtcctgcgccacctctcattggcctcaatctctctaagcttcagttgcccatctatcatatgggaataataatacagctcttgggttgttttgaaatgagcttaaatgagctggtcagtggctgggcacggtggctcatgcctgtaatcccagcactttgggaggctgaggcaggtggatcatctgaggacaggagttcaagaccagcctggccaacatgatgaaaccctgtctctactaaaaatacaaaaattagccagtcgtggtggcgcatgcctgtaatcccagctactcaggaggctgatagaggagaaccgcttgaacccaagaggcgaaggttgcagtgagccaagaacacaccattgcactccagcctgggcgacagagcaggactccgtctcaaaaaaaaaagagctggtcagtgtcaaatgcttagcacagagactggcacagtaatcttcaatgtccagcacctattgttactattttttttttttttttttttttgagacagagtcttgctctgtcgcccaggctggagtacagtggcgcgatctcggctcactgcaagctccacctcccaggttcatgccattctcctgcctcagcctcccgagtagctgggactacaggcgcccaccaccacgcctggctaattttttgtatttttagtagagacggggtttcactgcgttagccaggatggtctcaatctcctgacctcgtgatctgcccgcctcggcctcccaaagtgctgggattacaggcgtgagccaccatgcctggccctattgttactatttttacccctcacttctgtacagagcatttatggctcaagaaacatttgtcattttaattgtatgggagtcccacaacagcatagggagacatttctgatcattattcccattaggagggtggagaaactgaggctttgggaggtggtcctgacctagggaatcaatttgctgactcactaacccatgaagctctacagttaaaaaagactagattaaaaaatgagaactcagtaaaggggctgaggcaggaggatcgcctgagttcagaaatttgagatcagcctcggcaacatagtgagatcccctctctagaaaaattttttaaaaaattaggccgctcgaggcagagtgcagtggctcacgcctgtaatccaacacttcaggaggctgaagagggtggatcacctgaggtcaggagttccagaccagcctggccaacatggtgaaaccccgtctgtactaaaaatacaaaattagccggtgtggtggcacacgcctgtagtcccagctactcaataggctgagacaggagagtctcttgaacccggcaggcggaggttgcagtgagccgagatcgtgccactgcactccagcctgggcaagacagagcgagactccgtctcaaaaaatacaaacaaaacaaacaaacaaaaaattaggctgctagctcagtggctcatggctcacacctgaaatcctagcactttgggaggccaaggcaggaggatcgcttcagcccaggagttcgagaccaggctgggcaatacagggagacacagcgcccccactgcccctgtccgccccgacttgtctctctacaaaaaggcaaaagaaaaaaaaattagcctggcgtggtggtgtgcacctgtactcccagctactagagaggctggggccagaggaccgcttgagcccaggagttcgaggctgcagtgagctgtgatcgcaccactgcactccagcttgggtgaaagagtgagaccccatctccaaaacgaacaaacaaaaaatcccaaaaaacaaaagaactcagccaagtgtaaaagccctttctgatcccaggtcttagtgagccaccggcggggctgggattcgaacccagtggaatcagaaccgtgcaggtcccataacccacctagaccctagcaactccaggctagagggtcaccgcgtctatgcgaggccgggtgggcgggccgtcagctccgccctggggaggggtccgcgctgctgattggctgtggccggcaggtgaaccctcagccaatcagcggtacggggggcggtgcctccggggctcacctggctgcagccacgcaccccctctcagtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtaccccggatcccctgacttgcgagggacgcattcgggccgcaagctccgcgccccagccctgcgccccttcctctcccgtcgtcaccgcttcccttcttccaagaaagttcgggtcctgaggagcggagcggcctggaagcctcgcgcgctccggaccccccagtgatgggagtggggggtgggtggtgaggggcgagcgcggctttcctgccccctccagcgcagaccgaggcgggggcgtctggccgcggagtccgcggggtgggctcgcgcgggcggtgggggcgtgaagcggggtgtagggggtggggtgtggagaaggggtgccctggtgcaagtcgagggggagccaggagtcgtggggacgatcttcgagggaaggagaggggcatccgtagaaataaaggcacctgccatgccaagaaaggtcgtaaataggagtgagggtcccggggataagaaagtgaggtcggaggaggtgggagcgcccctcgctctgaggagtggtgcattcccggtctaaggaaagtggggtactggagaataaagacatctccaataaaatgagaaaggagactgaaagggaacggtgggctaggtcttgagggggtgactcggcggccccctcccgggagttcctgggggctcggcggccgtaggtttcggggtgggggagggtgacgtcgctgcccgcccgtcccggggctgcgggctggggtcctcccccaatcccgacgccgggagcgagggaggggcggcgctgttggtttcggtgagcaggagggaaccctccgagtcacccggttccatctacctttcccccaccccaggtctcctcttggctctgccaggagccggagccctgccaccctggctttgacgccgagagctacacgttcacggtgccccggcgccacctggagagaggccgcgtcctgggcagaggtgagggcgcgctgccggtgtccctgggcggagtagggaggggttggaaaggggccgagaaattgcactcccacacccctgggttgcaatgggcaagctccctccttggctcaaacgacaccccttggaatttacgcagatttggggatccaaacgtttacgactgaacactgtggtggagggggtaaccctgcctggttgttgactatgttataaagaagaggaggttgagggcacttactagaagccttcgtatttctcttattgccctcagcactgtagagacagtctctcatttcagactgcaaaaacctggtgggtgggaaggcctggcaggagtattcccgttttacagttgagcaaaccgaggctaagagagtgacgtccacttgctcagggtcagcagagcagatatctgaactgggatctgactcccaggccccattaagcaccatctctgggatgactgaagagcagccctctcttggttactgggctgtggcttttgtttacagatataaaagcttttttttttttttttttttttttttttaaagacagggtcttgctctgttgcccaagctggagtgcagtggcacaatcatagcttactgcagcctccatcctctgggctcaagtgatccttcccatctcagcctccctagtagctggttatcatgggcatgtgccaccacacctggataattttaacttttttttaggctgggcgcagtggctaacgcctgtaatcccagcactttgggaggccgaggtgggcggatcacaaggtcaggagttcgagaccagcctggccaacatggtgaaaccccatctctactaaataaaaatataaaaattagccgggcatggtggcgtgtgcctgtaatcctagctactcaggaggctgaggcaggagaattgcttaaacccaggaggcagaggttgcagtgagctgagatagtgccaccgcactccaacctgggtgacagagcaagactccgtctcaaaaaataaataaaaaataaatttttttttagagacaggagtctctccatgttgcccatgctggtgttgaactcttgggctcaaaggatccttctgtctcagcctctcaaagtgctgggattgcaggcctgagagactgtgcctggaccaaaacatcttgatacaggtactgtttcccccaaggaataatgatttttttttttttttttttttttgagatggagttttactcttgttgcccaggctggagtgtaatggcgtgatctcggctcactgccacctctggctcccaggttcaagtgattctcctgcctcagcctcctgagtagctgggattacaggtgccaccaccatgcccagctaattttttgtagttttagcagagaaggggtttcaccatgttggccaggctggtctggatctcctgacctcaggtgacccacctgcctcaatctcccaaagtgctgggattacaggcatgagccagcacgcctggccaggaataatgatctttttttaaaaaaagcatggtagttctgactcaggatgaaaatgcatacacccaatgtacttctaggccttatcgttgtgacaatctagagtttgctttgttccacttgactgttgtccagggagcaagggaaggggctagtttctcttctggtttcactgaatcccaagtgcttcctttatatcaaaccactttctcctctctcatgattttacttgattgggtatcacaataagaggcattttttttttaagagaaatcacccacaattcccctgagggaacacctccactgtgtcattcttccagtctttgaccacatgaatctttagcaatcacgctgttgtctttacattttttttttgaactttggaagtcattcgaagttttttacagagaaactacagctcgactgatgaagtatatttgctctgtgtgctctagagctttctattggatcagaagttatctctaactcctgggctcaagtgattcacccgccttggcctcccagagtgctggggattggatattttgatgtcagtgggcattgaggagtggcttctcggggaaggcaaacttgatccagtcttggagagcaaggcaggggctagaaacaagcttgtatctgagcatgcgtctggggtgtcccagtgctaaggctggtgactttgttttcctgcaccaagatatttggtagcaccaagggtgggttggtctggtttttatttctcccttctaagctcctgtgtttgaccaataatattattacatattaacactaatatgttaataattaatagtgacacgtattcacttttgcctcagactctggggcaagtgcttcacatattttacctcatttagtcttcactagaacgttaggagggtgggcactgttattcttcctactttgcaagtgaggaaactgaggcataaagcaattctgtaatctgaagcatccgcagttggtcttttctcttcctttttctccttgtttttcacctcaataacctgaaaaaacaaaaacaaagaaaaaaggtgtttggggaattagtgtccaatgtgaagtaagctgagaatggaggtgttgacagggtgggacctccgagagggggtggtgatgatggagatgggtcagacccgggggaggtgg

SEQ ID NO: 42 - DNA, 8000 bp, Homo sapiens
  RASSF1A DNA region
gggcggagctccagcgaccgcttcccctcccgcccgccggcaccccctggctcccacctggtcccggcgcggcctgcgagctagcgaggttcgcgcggtgaagtactgctcgagctccgagtccgagtcctcttggctgcagtagccactgctcgtcgtgctgctccaggtcatttcgaaagaaggcgcctccgcctcgcccatagccgtacccgcccgtcccccagtcctgcgcgtccgtagccgccaaccaccgccccggtcgcgtgcgtgcgtgtacgcgtgtcagtgtgcgcgtgcgcccgggccagagccgcgccgcaaccgttaagactgaaacgtagatcgccgggatctagctcttgtctcattggggcaggaacgccggggcggggacacgcacgcttcgcccccaggaatgacctcatcgctccggagctccactcacagaccccacctaccacagggaacgggggcgggtgccagcgtccgggcaagcgcacaagagtggcctctggccggaggcgagggcgggaaggtgcgggaagtgcgcgtgcgcggagcctgggtcagcctgggcccgggtccgcttgcagcgggtggagtacttgcggagccggcaatccaggctcccctcccagcccccgcgcagaattagcctctctgtgccgccgggaaatcggcaattagaacgctccttgcgcgcggcacccaggcagccctcgagaatgcctgcactgtggcctgcccatcctcgcccttcccatacgccctcggccccgcgctcaccacgttcgtgtcccgctccaccgcgggttcccagcccaggtcccggggcccgcaacagtccaggcagacgagcgcgcggcagcggtagtggcaggtgaacttgcaatctgcagagaggcctggcggtgaggcggaggagctccaggtcggggaaatgtcccggagattgaagggaagccccagggagagggccgctgctcgccaggctccgcaggcccgacctatctcagtgggttacctcacactgctacgcggactctaatgttggccacctgggcgtctggaaaccggccggaaggccacaggcagagaggcctgctcaacagttggatctctatcgcctagcacagaacttcccctttcctcattggcaattaaaaaaacaacaacaaaaaactgcgtcttgcttttgtcacccaggctggagtgcaatggcgcgatttcggctcaccgcaacctccgcctcctgggttcaagcgattcttctgcctcagcctcctgagtacctaggattacaggcgcccgccaccatgcccagctaatttttgtatttttagtagagacggggtttcaccatgttagccaggctggtctcaaactcctgatctcaggtgatccacccgcctcggccttccaaagtgctgggactacaggcttgagccaccgcacccggcccttcactgggaacgtatatggaatacatctgcccatttacttgaaggaaaaactaaacacctttaacctacgtctgccctgtggttgtcacctgtctctactcccctcagaccaagacactggtctctatacactctaatccttcgccttcactctcccctctacccactccagccaggcttgcctcttcctccaggaaactgcccgggacagggtcctcagcgatctgtgtactaccaaatggaatccagtgttccattctccattctcaccccctcagcatcatttgaagcttgctccctttgactcccaggggctacactctcccagttttcctcctaccccctgcagctcctgctcagctcctttgcagattctgactcaacttccatatctcacgatgaagtctgggctcagtcctgatcactggcctggtctgtctacattcatctgccccagatccacggctgaaacactgacctaaaccctcagactagatcctccgtgccagtaccttcactaggatgtctaaaagacgtttcaagtgaacatggccaaaatttaattcccttttcttcagcctcactgctacacttgcccagcttcctctttgcagcaaaaatggccactaggctcccagttactggagacaaaagcccaaacttatctttgatttctcccttgtctctacctctgataaacatgcccaaatcatcctgcttcttatctccatggctactttatttctctttgagaacgctgcaatgtcccagccttgttctttttttttttttttttttttttgagacagagtctcactctgtcgccaaggctggagggcagtggcacgatctcggctcactgcaacctccgcctcctgtgttcaagcaattctcccacctcagcctcccgagtagctgggattacaggcacccgccactacgcctggctcatttttttttattttttagtagacatgaggtttcaccatgttggccaggctggtcttgaactcctgacctcaggtgatccacccgcctccgccttccgaagtgctgggattacaggcatgagccaccgcgctcggccccttgttcattctttgcattctgtcacaactttgtgctccccccagctgaatttgtgatgtcctcttgtaccggatgagagggtctccatgcacacacagacctgggacactatccatccacaagttcctaaataggccagagcagtgatgctcaacccagactccatgttacaataatttggggagtttttaaaatttactgatgcctagggtccactcccagcagttgattcaacaggtctgcggtgggatccaggctagcggggaggactgtaaaagcacccctggtgattccagctggtgtctacccaggggagagcaacctttgcttgctggcgattcccaggggtgcagaaggactgctgggtgtgtggctgcgtgcatattttagcatctgattcactgggtcagaaaagggtgtttgctaaataaagactcaacaaaactcctgcttgcagggggcccaccaaaggttctaaatttttccaggctccctcccataggtggtaatttcccttcaccctaaaggttctggagggggtcatgagtgtttgagaagaggcaagcctgggaagatggactccgaggacagtaggcacaaaccctttctcaagaagggccaaggcattttaaagataagaaacttaaaatcagcgtatttttacatataagcagccacctctgctcatctgtggcccagatacgagtggagtgcgacaagggataaaccattttcgcgcactcttcagcgatggggcgaaagtaacggacctagtcctcgggagctgtccccgccgaccccctctgccgcgacttgacccgcggcgactgcgctgccccttggctgccccttccgctctcgtaggcgcgcggggccactactcacgcgcgcactgcaggcctttgcgcacgacgccccagatgaagtcgccacagaggtcgcaccacgtgtgcgtggcgggccccgcgggctggaagcggtggccacggccagggaccagctgccgtgtggggttgcacgcggtgccccgcgcgatgcgcagcgcgttggcacgctccagccgggtgcggcccttcccagcgcgcccagcgggtgccagctcccgcagctcaatgagctcaggctcccccgacatggcccggttgggcccgtgcttcgctggctttgggcgctagcaagcgcgggccgggcggggccacagggcgggccccgacttcagcgcctcccccaggatccagactgggcggcgggaaggagctgaggagagccgcgcaatggaaacctgggtgcagggactgtggggcccgaaggcggggctgggcgcgctctcgcagagccccccccgccttgcccttccttccctccttcgtcccctcctcacaccccaccccggacggccacaacgacggcgaccgcaaagcaccacgcggagatacccgtgtttctggaggccagctttactgtgctagaggaagagggtccccacatccggccctggccctcctggtccggtttgctgaagcaacacacttggcctacccactgggtggggcaggaagtctcgagccttcacttggggtgaggaggagggagatcggtcagcagctttaccgcccgctctgctctccactgcggagactggggctccggcagaggctggaccgtgatcttgaggttcaggggtgcattctgggtggattcccttggcatgggtggtcggccctcagcaactgcagccctcatttggctctgtcaccctgggctgccaggacacaagtctttccatgcttttcccagtgcttgacttggcactccctgcaggcaggtgggtattgaggatggcaatgcatgtgggggatgtgggagtagggcttagaggtccaaggttctaggataccctcacctgcagcaataccactcattctggcatcgtgagcagcgcttagaagcctctgcactgcagtaagcacagcggggccgctctggagccactgcctctagcacatccagcctgtaggtctcagcccacctgggggaaagtcaggaaggtctgactggccctggaaggtgggggcaccccacccacatccatgcctcctgcatcccctccaccctccctgccatttccacaggccttaccttcgcgcctgcagccgcaggtcctgctctgaggggctgaacacatgctggagctggtgcttggcaattgcctgccacttgcctctgttttctcgctccagccgctcccagatttctgggatctaggagagagaagtggagagtggcaggaaggtgctggtaaagtgggacagtggtcctgagcagctaactttccagtgcctacctgttccaacaccaggtccttcttaggaggctgggtttcagttagggtcagatgggccaggaaactctgcaagtgggccaggttgggcagctggtccagcagtgtgtctgtgaggaaggcccgaagctgcaagggtgtccagtggaggccagtgtgatgcaggtgctggccagggggccaaggaaaaggaaggggctgggttgggctgagaatgcctgtgggggcccatgtcccactagctggtgtgtcacctgaggcccaaggactgaaaagagcactggggttgaggtgggtgggcaaagataaacagcagggacgcagtcggggtaggatggggagggcggtactggggctgtgggggctggtgcggagggagtctgaccttgagtagccgtcccttggcaaaacttgtgaggcagtagcgcgcctgagcctcagggcttagcagcaggttgtacagggcgatccacacttgcccgtccaacttgctcagcttttgctgctctgagggggccacagtatgccaacggctgccctcgaactgctgcagcttgcctggggagaggaaccagcacactgggtgcagacgttgaattctcttcaagtgagccagctgtgggggctgggagactgaccactgcctgggaatgggagaacctgggctgcaggacttgcccaggctatagggctgctgaccccagagcagggtgagcctcagtagtggacacatgggcctggcctgctgcagagctcaggtctaggccaaccttggctgaggcatcctggtatcctcagtgggcttggggacagggtttgtcttcgtgtgtgcaagctgcccccctctatcaaaaagataccctgaagcagtagcctggggcttaggctggtgggggaggaccctacctccttcccgccggctccagggactatgctccagcagttccaccaggaggcagggcaggttgtgtgtgctaagcatacggctcaaggtgctgagagagaggctaggggcagggacgttgtgaaggaggtgagtagaggcaggctacaggcccggtcttccagccctccacagtaggcccaggcccagtcggactgctcacctgtccacacagtctgtgatgtagcgtagtactgagagggccttcagtgcaatctcaaattccatcagctctgcctgcttctgcagctcctgggaggtcacacagtgttcacgctgggccaccctcacctgcgcaggccttggggaacccagacctaggctttcacaaccctaggtaacctcaacccacctgcatggggttgctgtcctgggatccctccccctcaggggggccaccacagccactctgggccaccagcagggtcagtttgcggtggcaatagtctaccaagtccaagacagtgtcttctgctgactcacacacctcctgggaaaaggaggagggaaactttctgtgtctgatgccttccccacacagacacaatctcccttcctgccaggccctgacccggaactgtggcccatgggcagagatagtccctcaccttgtggaagaacactgtctccaagaggttgatgatggaggcctcgtggtgcacctgtcagataaagagaaggacagggcctgaggaagggataggggctaccagctctcctccccggtccagagccctctgaaggagtgagaagaggtatgaaccaggggcccagctcaccaccatgtagatggggaaggtgttctggggcttgaagtcctccaccctgcagaacacagggaacaccttctgcttccacatctccactgcgatcagctcctccaccagtgttgggacctttgaagggtagggtaaaggacagggatgagatgggcctacctttgcctggcaaccctcccatccccggctcagctcaaccctgatatccccaaacactgctgggcccctaaactattccagacccatgagtatccttcctgacttctctcttgcaccttggaccttcattaccagctgctcccaacacctctgtggatgtccctcatgcttatgtgactggctcctgcccagttcccagactgccttgccctgcctgcacctgcacccttatcctctgcccgcccattcaggcatccctttcaagccagcccttctcctcatgccctccctctagtccctgtctgtggtcctccaaagctcttgtgtgccttggcctcactcctactcaggacccttcacagtgccctgttcctatcctatcccctagtatgagttccatctttcttttgcaaaggacctttcttttttttttttttttttttgagacggagtttcactcttgtcacccaggctggagtgcagtggcatgatcttggctctctgtaacctctgcctcccaggttcaagcgattctcctgcctcagcctcccgagtagctgggattacaggtgtgcaccaccatgctctgcaaatttttgtatttttagtagaggtggggtttcaccatgttggccaggctggccttgaactcctgacctcagatgatccacccgtcttggcctcccaaaatgctgggataacaggcatgagctaccatgcccggcttttttttttttttttttggtcaattgatatctcaggtcatctgctggtgacactccctggtccatgttccagccaggcatctccctggtttctaacttccccctttcatgttgggaaggctgcttccttccatgccgaagttccacctccagttaggttatctcccctccctcccgccctccccaacccctccccaccccttccccaccaccagccttacagtcaattcacagatgcctttagcatttgggccttccccttgaagcctaagctgttctaggacctacagctttctccctaatcaactcatgccctgcacccagcctggtgtgccagagtcctgtccaatagggatacaaaatgaaacatgtaacacattcctctttgtccttcagggagaacaggtgtaccaggccagaggggaagggcacacaggggactccggaggggtagagtaggtgaggtatgggggaaggcaggaaccctgcctgtgacct

SEQ ID NO: 43 - DNA, 8000 bp, Homo sapiens
  HOXA10 DNA region
tagtttattccgcttaccccagtcctcctaggcttctgtgaatttcagaaagcaaaaacaaacaaaaaaaaaactttttgttcaaggcgatttaaaaaaacaacttcacaagatagggagaattgtggtgtgcttgtcacatgttaccagtggtaacaattttaatgacaaaaaaatccactaattccaaatgcataaacaaaactacattatttatctacagccagaaggatatggaaatttagaggtaaatgaaacattttagtcaggcaatgtaagaccttacagaaactggaagagaagtccccttctcttggtaattctttttttttctttttaaagctgggatatcttacagaggaaggaaaaattaaccttttttactttctttctcactttttaaatcagccaaagtcaagcccgtttgccaacctgcatgtccatgcctgtaagcccttctcttggccaaggaagaaaggaagaaagaaaaaagaaacccaggggcctgtatcccctgattaaacacagcacagcactccaggcagacatgcccggtggcggctcctttgcaccattgacctcaggccagacacctcagcgccaacaatgggacctcggccttccggctaggtttgccccaggctgggcaggaaaccagctcggccgaagacaggggccatttcgagcagtgggaccccaagacagcaaacccagcccagtcaggacttgacacttaggacaatatctatctctatagaattttagcatgatatggctttttcccccagaaaacaacaaataaaccagcaccaagcaaacacaaagaaacaaaaagtcagaacaaaccagccctgcacagatgtaacggcccaggagatggcgagtgtgggagggaggaacagggctccagcacaggtgcgagttcctgggcagagcctgaagacagagggaggggaccagcgctcgggaagtgaaaaaaccgcgtcgcctggagattcatcaggaaaaattaaagttggctgtgagctcccggatccggttttctcgattcattttcttcagtttcatcctgcggttctgaaaccagattttcacttgtctgtccgtgaggtggacgctgcggctaatctctaggcgccgctctcgagtaaggtacatattgaacagaaactccttctccagctccagtgtctggtgcttcgtgtaggggcagcgcttcttccgaccactctttgccgtgagccagttggctgcgttttcacctttggaattgcctggcatgtaagagaataaagaggggatgattaagtcgaggccacacgggctgcccgcgggggtgaattgcctccgtttctcccataagagagatgtcccaagtcacagagaagagagtcgtgcccccgtttttggcttgctgagagcaagcagttcctccaaaagtcatgacaaaaattgagtgggccttcaatctacatgacccttccaatttacatttcccccacttttccaaacacctgttttagcgctaagccgtagatgcttgcagaaggaaaggcctgggagggcaggctgtacatcttgaacttaacgcttttctttgcctcttgccatatggcagacaagcatttcctgtagcccccaggctaggagcgcggggtgcttacttggaagatgggccaggcagcttggctgtctcaccccagggccctgattgcccaagactcgataaggggagaaagaagggcatcattggtccaatggggaaggcaggaaaaaccgattcgggggtcaagggccctccctcaagtttctccaggagccagggatagatagctgggcgattccgaggtccaggggaagggaaatggcccttctctggctctcagctcagggccccccgcttccaggccggatttgccttttcttcttcccgcaacgaagattcccgcccctcagcaactttgaaaaaagcatgggggatcgtaaactcgaacttcgccggttaatgggcttatttattggcgctggcggctgcttattttggatgccttacaaacatccgcgctatctgcgggcgagctactttccctccctccccctccccccgcgtgggccgcgccgcgcaggctgggcagggaccagggctctgggtcctcccggccacaggaaagagcgcacaggagggggcctgctcgctggtgtcctcgtccctagtcagggggagctgaggccagcgccgaggacgtcttgctgtggggcgctaagccggacatgaattttactgcgtccccacgcccaaatattaaaaagcaagttcacaaggtcagcctgcctgcagcttgggccaaggccggccggctgctgcgcgggctcctagttttctgatccttctcctccttgtgtctgcctgtctgcccgcctgactgcagccctctgcagccctgcttacccagggaatccttctccggcgaggctttgctgctctcggaaggggccggggagagctcctccgcggccgaggacgacgcgtgcgcctcctcgtcgccctgcgagcccccgccgctgccgcaagccagcgtggggggcggcggcgaatcgagggctcgctccttccgggccgcatcggccgagccggaggctagcgcgggcgggagatcgaaaccgcgccccgggggctgcgcggggaacgggccagccccgagttgctgcgcgccgccgccgccgctgccatagcccttggcggtgccgtaggcctgagaaaggcggaagtagccaggcactggcaccccgctggaggtgcccagggcgcagccgtcgggcggcgggccccgcgggaagggagccagttcggcggcggtggccgagactttggggcatttgtccgccgagtcgtagaggcagtaggagctctcttctttgatgttctgcgcgaaagagcacgaggtggcctgcggcgctggctggggtggttgcggcgggggcggcggctgctgctggggcggcggcggcggcccgtcaggcggctccatccggcaagaccggggcgcgtctagccacaggtctatgggcgagggcccgtagccgtgcgccccgggacctagacccccgccaccgccaccgctgcccggcgacgctgcctcattgcgcttgccgcccagcgtggggaagagcccgcagctctgcagcccgtagggcaggtcggcggcgggcggcaggtagaccccgccgtgggcgtagtaaccgccaccgccgccgccccccgcgccaccaccaccgccgcctgcctcgcctctgcccgagctgatgagcgagtcgaccaaaaaagagttcgcggcggggctctccgagcatgacattgttgtgggataatttggcgaagggagcagatagccctttctggctgacatttcttgtgcaaaacatgctgaatacgattagcaatccccccgcaccgcggcgggcgcccgcagccaatcccgagccagagtttccgcgcgaccactcccagtttggtttcgtaggcgcggggccgctctccgagggcgccctcagagcccgcgattgatataaatatgtaatctgtattgatgggccaggagacgcaccccgacaccttggcccgaaggccgggagctgtgggggctgccccaacgtggctggtggggggcctggccattgggctcgccccgcccctacccggacgtgagccccataccggggtcccttagaagggcccttgggccccgcgcagttaacaagtggggtgtttatggtgcgcgcccagtctgccttgggtgctcaccatccctgtcgcagaagctgccactagtccccggtgtactctaaccactgaagcggccgtgtcggggactcacgcgcttcccattcagctctggatctggaactggccccttgtctgaattctgcctcctcaaaagtggcgaacctggccctatggccgtcaggatcctcagagtgtcaggagcccagagtgaactagaagctgacttgcctctacttccagtatccacagatttttccccaaaatgcagtggttgttccctagcccctaacccccaacacttttctcccctagtccgttgatccagttaggatcttctcctctggtgttgtgctcacccggcttgccttctctacaactcctgtcagtggactttggtgggctggcgggactgggggtgggaagcggctgccagccttgatggaaagacggccccactcccactcccaaggccagagccagatccaagcatcccctcatcacctagggaagactctaagcccacagtcatttcggggaagtaaaatgctggccttggggtccttggctggcccaaagtgagagacttggaggggtgtgcccggtgtacacctccgctgctttatgttcctgctctcctgttagctttggcactcatatgctcctggaggctacaacaagaaccagtgtcactacactcctacctctctgtcccctcccctcttcccctccctcccataccctcagctagggcagtccttgctcctgcagtgtgtgcattttaacgaggcctcggtgaggaaatcctttccctgcaaggccgggctgggctcatggtcctgttataagagcatgtggaggtcaggccctttgctcagcaggctcaggggctctgttttagccaactggaaatggccggcctggggtgctagcccccaagtgtgagcctcatgctagtctcaggaaatgcaaccttttccggccagatccagtggagcccgggaggtcgctgccttgcccctggcccgtgggctacaggtctccaggatactctaggcctgcccgcccctccggccccagtcggagctggcattactgaacgccggcatcccaggaaatagaccgctcaggccgctgccttcagctgggaaagactcttgcatccgggtcatacgcggcactttgcctccttcctcccagtgtgaatccagcccaaggtggaagaggagcctgagaggaccctgaaagcgcagagagagctggctaggtagagctccagctctggcttctgagattcaagcagctgcggtcgctgcgggcagtggctgctccgagctccgggcccagaaggccgctccaccgctagcgcggcgctccagccacttcaacctcggcgccccacggtgacccggccgtaaggactgagcatcagggtgcggaggaggaggatggagagaaaaagagagagatagaaggagagacggacagagggagagaaaagagagagaaacaggaggggagagagaataagaaagccagttctctccagcctgacagggtcttgaagctgcagcagcggctttagagggatcaagacagggtctgtgccaagagatcattaactctccttcgctcttcgcatgctcctgcgcccaaggccaaggcaaattctctaccctctaggcccatgagacggacgaacctgagtgcagaaaagcttcagagcaccgcagccagtggccctatctttagcttcccggatctgcctggtccttaccctgctcaccagaagggtggaactggctgggactgttgcccttagcaatgccacctggaaatgccacagagctgggggctgcctaggaaggaagctttgctcttcagcactgcccatgcctctcagaaaagctgtgtcatttggcaagtgggtcccaagaccttcgaggtttcctcaaggataatgagccgtggcaggtcagtgggccaggacctgggcctagagtcatgcctctccaggctaaatgcagaaagcaacttcccaggccgacatctgggaaggtccagtatatatttgagatagtggaggctatacaaataaagtaaaagacaatgatcttaacctatctttcctaaccttttagggagtcaggacccttctggaaaatccaagtaagttttgactcattctgtgaaaaatgcatatctgtatattcccacaatttgatccatcatttcgaaggagttcacacacccctcagtcttttccttgggtcttccagagtcccccagtgtcctgggacttgggggccctgccaatttggctggcaagacaagctgggtgccctgcagcagacagctcaggtcagggccccgtagggcagccaaatgggcttctaggctggatagaatccagggacctgcacgtgccaaccaaggttacaaaagtcagtttgtatcgcatctgctacaaaaggctgccccagccgaagagtagaggagctgggaggcctgctatgggcctcgcaagccgtggcgttggcacttgggtcttcagtgacctttctgtggtgagccagatgtgtttctttagtatgactttttcccccttgtaataaaccaaacgggatgttgggcttgagctccttctggcttttaaaaagagtgtgtaagctcatgtatggcagcccctctctggcccaggactgccttccaactcccaccagggcttgctgagtgggaaacgcagcgagacactgccggtgagctggccggctgcaggcgggccggctgctgctggttcaaatgagaggatactaacgcagcagaagctcaagttaaacgcctccttggccctctcagttggtctcctagcccccagcatccctgaccgacccccaaggctcccatgctctctagactttaacactggagtgttaggtttctcaataaaatattccatttattgagcgcctcggctttcctgagattccccaccccaagagatgaagaaagcgattcagttgtagtgtgggtcggcttccaagcacttttcctccctcgtgctcatttgccttctttcttttttgccattcttcccagctactaaatttctccctctccctcagggtaactttgacctaaatcttcagtgcctgcttgcctcaaccaggccttatgcagcggttacacagtcacctccagagcccatgttttatttaggaggaaatattgcccatttctacccgggtaataacctgccagccctatggggccaaagtaaacaccaggcccatggattaaggccttcggcaaggattaggctgcagttcactaggtactccttctgagtaggcccttgtaagcagtgagaaataaactattatgaaaaacaagttcctaatgaaaagataccaggtcctgagatcgaaactgctagtaaacaatttatatctggagggcactctcaatgctttcaagactctgaattatccagcggtggagttgggctgcagtcccacagaggaaaaataaaggctgcttttacatatctggctttagcaaaaaaagaaaaaaaaatcagagcttctcatatccatgcatttatgcatagttagaaaatttttccacaagcaatgatgttcttttcccattcatctttaagaatgtcaacattttcagggatatttgatgtgtctgttttgaatctatcaggtgcaatctattactaaacaacatacttttaaagttttctaaatccaaccattaagatggcccttcccctgttagggaagtgttggggccttttcacacaccctttgaattgcaatattcttagatttgttcataataaacagcctctggccttagaactggaggcggctctggcattgtagggagggctcccgctgcagagagcagctcactgggtgggcgctcatttcttgccttgttgctttgctccgtcatagtttgggagccagccttcaacaaagactagtaactatgataggcaaaatttacgcaacaaacaggcagtgcctactttctctgggattcaccccactcaccacgcactgtatatttgggaaaagaaaaagatgtgggaaactaatggaaaaatagtagaaaaaattaaatataggagaaagagggaatttgaatcgatccttcaaaatgtaatggaattttttcatttaaaatgtgtttgatgacaacccca

SEQ ID NO: 44 - DNA, 8000 bp, Homo sapiens
  ZGPAT DNA region
accttattatttttcagaagtttattaactgaaatttagtgaggtgtggtggggagagcctactgaactctgagaggttagcagctaaaatttgatttataactgttggaaccaagaggagaggaatttctttgcaggttttcaaatcctgttctgcctgtggtggtgcagaattccttagcctcacatttcccataccccagacccatttgggattcatcaccggttgactttttaaagttaatataaaatttgtcaggttaaaaaggtataattttaatcgtttggttcaaatcatccacatttcatagttgagaagggtagaaataacacctcccataagatatacacattactcatcagattcactttgtgactaaagttaccaagccattggccgccttaaatacttaaagccatcctgaggacctctgcatctgtcaggcaaggctgaggcagttgagcagccagacagaagcatctatccggcccccaccctcaatttggcctagaaagaaaaacagtcattgaaaaggtctcaggctggacaaacacagcaagtcctctgtcaatggggatgtctgcccacagaacaagcagttgtttaacttgttgaaccacaggccaaatccaagtgggactctgttatcttaaacggtatccagttctctatgcacaaagatctctctctctctctctccccctcaacgtccaaccagaattccaactggcttggcggcttttacccttccatctcccctcttttctaggtccaggcctaggaggcaggggggtagactcaccttttcccagagggtattattattttcaattctttctaaccaaaaggcattcttccccaaaggtaataagctccttccagcacaaatcaggaaagaaaggaggcctaactttgcattcctacagaagggcggcatgctgggcgtcctcgctctgaaatttcctcatggcaggatgtctggcatggaaaagtcctggttggttggcaataatagaatttactcttttgcttctgggggaaatgggtgtgaggttccctagagatccctgtccacccataaggcagccccaacccaacttctaagaggtctggatcacttctctcccaggtcctgtcagccttttgtgcactacagacccatagacatgcaaacaaatattgcagagattggactagggcaggcaacagggaccttaggaggctctctgtagcgtttccatcacacagcctacacagggaactagggtaaacatctcctttagttcaaactctgacaccacttagcaaacaagccagtcatcttgctgaaagagaggtgagagaatttgggagcaatcctggggcccgtggacctgaggtcccaaacaaccacggaatgtagaaactcagtttcgttttccccaaatgcttgctccccacatcaaccacggcctcctaattaccagtccatagcacatcctggagactttttggaatctgccctcctggccccaccttgagaagaggaacgggtgccagggagctccagtgattgatagtgccggaggagagccaagaaggtgggacagtgggtaggtgcaaaaagcaccatttttatttgggggagcctcggaagctgcaaattgtggggccgacctagccggcgcaggactacccaaggtcagaggtcagctgcagctagagacttcagttcccgagctgctccttactgactagccggcaggcgcgggaaaggcatgcccacctctccgctgcaacttgtccctggcgggcgcagagcaaaaaccctgaaagctggcagggagctctgagccagagaggaaactggccccggcctttgcccgctgcctgatttactcatctgcaccctgcacctttctggctcccctccattcgaccctcaagcgccaggagtttgctgaggctgggttgtgcaggggcctttactcattctcctgttctctagagggagaagcttgggtttcccagctcggggctgaagtaagtgtgggcagcatccgggtgcccagaatagagtttgtatcagtagatcaggctgggcatttagccctccctttgaatcacccctcccccaacctgcaggcggctgagacaggagaaaaaagagcagcatgcagtggtgtgggtcgctgcgaggacaatactgtcggaaagcggggaaaacatgaccttggaatccaaagggagtttcctgaagacggtgacggccgaagtgcaaccctccaaaagcccctccgctttcctaagactttgacatgggccattcatcaaccccaactttcagatccaacctttgagccagtcctagctttctttcacagctcagctctattccctcctcattggcccctaatttccaatgcccctgggtctaggctaggggccagaatcctaggagctgcggcctgctccccagagagctagttgctgctcttgttcgcattggagataaagaaagccaagtcccgaacccactgctggccgcgcgcagagagcagatcccagcagtcagcttcctgaggcaggaccagggtgagggagggcaaggagccgccctgaggtggactcaggcggcatctgccgtcaggttggggcggagaagttccatcgtcttgctgccatctgggctaacttggcgctttgaccctggagaggggaagcagagactctcctagccacccccttgggggcagagcagagatacctcctgtaaaggccccctcccggcacccgccttgctcatttttcgcgagggaagctctgggccacgacttccattcttgtgcccacggtggggcacccaggcaattcacaaggagagaagggaaggcaggcaggcaacaaactgctcctggttcctgtgccagtgggagccccatttgtaccccttttccctcctcctgggaagaagggatggactcatcctctgcgttgcccctttgcggaagggccccctcctccccttagtgtcagtggggtggggcctttatccacggttgggggaggggggtaggatgaaggaggagatggggcagaaagactggctgggcctcttcccccactggggtttgggtgccagtggtccccttgagggtacttgctacccggtcgctggaggtacagttctcatctgtctgccccctggactcacgcacttaccccactccacctgttgccaggatcgcctcatttctgcacatcatttcggggtggctgagaggggaccacaaacccctccaagcctctcttttgtggttccagcgaggcggtcatccttcacttctccttttccagctttcaggaagcagttagggaatgtatgatgaagcagaaatgagccgtcaggatgatattttaatggcttatatgtcacgttggtgcatgtggctttgaactggagccgttcgcgtttcctgcagagagcgccgcaaatcccacttgataacttctacaacccacaacttttttttttctcacattcactcttaccctgtatatttggatgtgtttcctaaaaataccctcttgtcaacgtccccgttggattttccacggactggagaggagaagactgacttttcttttttgaagaattattttccctctccccctcccaacagctacccttccccccttaggtcccctcccttgttgtgtgtgtgtgtgtgcgcccgcgtgtgtttcctgaaaggcgggcgaggtgggttcaatatctgttctccctagtgagaggaatacagcctctagagaaaacttctccatacagatgaggttttctaaactccaggggaagcaggatccaacttccccttgtaggtaaaaagacttagtgcctccgatatatcttttttttttccaaccaagtgtacaataatttttaaagatacctcggccctttctttacctccactcctcattccattccactcaaagttggtgggaaatgctgggctgctagactcagacttgttgatgggaacagaacaattaattttttttccgaatttatatttcccggcacaagcacaaatgctcagccaggtcccttcaggcaccgggaaatcatcccggatacccaagccgacttttgagcaagcacagcccatggaaagggcagtcccgccggccagccccaagcgagaatctagttggtgagaagaccagaaaaccagaaaggcgaggagcggcggacgctgaccctgccttcctccagcccgtgcagtcagcgctggcgtcagggcaaaaaatatattcattttcattttcctctcgctggggcacggtgagtttcctaaccgggccgcctatgaaaggatgagttgaggtttctttgtttggaaaaagagtttagggctttgattcagctgcaaagaagccaaatgaagttagaaacaaagggtaaattgaaggattccgactcttggctttttgtgttttccttactagaaaataattagacctaatgaatatgcagacgcttcagctaaagcctcggccaggactgctgggttttaaacagagctgcggagaaatgcaaccttcgtccccacaaccccccgcatccgcgtgcattttttgaaatgcataaatgttgctcggcttaattgattgagtcacagggatttttttttcctgcttttaagtaccatactcccctacctttcaacaatcattttaatatatagtcaatggctctttgtggaagggacaaaaagaaactacgcgcagttgttgaatagactttgcgctaggcaaagacaggttaatcgagggccgcatcgcgaaaacaagcgagtgttgtatgtttgcactgaatccaaatgtctgcattttcctaactaaatcaaagggagtgttatttctttttctgctcactcatctgaaggtaagtaaattttctctggcgatcaaaagcctggtcggcaacaaagaccgcgcccgctttttccaccgtcctggtgtggcgagttgcggaatttcaataactgtgacaagggtgactgatttgtgaactagaaaaggtttgaatgtcacaaaatttacattggtcctatctattggggatttaaataccgaaaaagtattcggggatctctagggagctttgggcagcccaagggaacaatagcgcgttgggaaatttacagttttacctgaatgaaaggggcccaagtagggaacaggagtgaggagagacagggttagcgggggcagtcgaaggagacaacggaaaggcagaaaacagaaaaataacgcaagagagagaaaaagtaaaggaaactggcaacaacgtttcaaatttgccattaaaaaattcaaacctcaataacctgggaacttaagcgctttcatccacctgtgtgtgtgagtgtgtgtgtgtgtgtgttttcggggaggggggtgcggtggtgccggaagtctgattagataaaagcgagaaatacagaggctgccggttacactgagaaattacatttttttgtaaagagagcatagtgcccgtgtgctcccaagatcttaaattataaacaggagggggacaaagcaagagggaaacaaacttcaaaaggagcaaataacaaaagcctcttttgctgctcttgaagggaggggggaagggggaagaaagaaaagaaagttgctgaatcgggacattctggaagtgcctttgctgtgtttactagccccatccccgcctgctgcacccggagacgtctgaagtctctaatcgctcagcgaaaagttggtttcgggaagggcagcgccggggtgcgggagggcaaaggaggaagaggaatgcatatgactgtgagaaggaaaagtaatcgtggcaaaaaaaaaaaaaaaaaaaaaaaaagaaagaaagaaaagaaaaaagaaaaaaaatttccgtgagaagagggaaaaaatttggctaaaaaaaaagttgctactcctggcagccctggtttgtcaaaaggggatgtcaagcgctttacaatacctgggattgatgaggcgggcgggccaatgagctgcgcgcggcgcctcggcgcgccctccgttggcgcggcggctgagggcggggggaatgcgggcgcaccaatgggcgccagcgtcggcagcacgtgacacctcccccctgctcccattcatcaagggggggacggtgtcgtcctttcaattcatttatctgcaggaatgattgctgctatcagtctcgcgctcaccgcccggctgaggaggtgaaagtttctccccaggaagataaaccgcaaaagacaatattgtgcatgatttgcgccttttctttggctttttctttctttcttcacccccccacccacttttttttttttttttttcaaaaagcagagagggaaaaacggagagtgaaggagcgaggaggcgagcgtgagagaaaggagagagagagaaaagaaagggcgaggggctagtggagaagtaaggaggggcgcgctgcgcgaggcggagagagggcgaagcagtcgcggcactggcgctcacattcctctatgctacaaatccaggaggaagtttttttttagggggctgagatgctccatgcctttccccgggcagccttgacgcgcggccctctcggcagagactgagcggcgagaaagtgcgagccgggccggcagaatctgcctggcgggcgctggagcctgcgttactcgcggcccgcagccgtccggctactttgcgtttggcccggccagcgcccgggcgcgccgcgccattgcctgcaggctaggacttcgcgaggtgggtcgactccccctccctcctcctcttcttcctcctcttcctcctcctcttgttcctcctcctcctcccgattttccctcctcggctggcgagggtggggggggcgggggaggccggggctcgccccgagcagccacgatgctcctggacgccggcccccagtacccagcgatcggcgtgaccacctttggcgcgtcccgccaccactccgcgggcgacgtggccgaacgagacgtgggcctgggcatcaacccgttcgccgacggcatgggcgccttcaagctcaaccccagttcgcacgagctggcttcggccggccagacggccttcacgtcgcaggcgccaggctacgcggctgctgcggccctgggccatcaccatcacccgggccacgtcggctcctattccagcgcagccttcaactccacgcgggactttctgttccgcaaccggggttttggcgacgcggcggcggcagccagcgcacagcacagcctctttgctgcatcggccgggggcttcgggggcccacacggccacacggacgccgcgggccacctcctcttccccgggcttcacgagcaggctgccggccacgcgtcgcctaacgtggtcaacgggcagatgaggctcggcttctcgggggacatgtacccgcgaccggagcagtacggccaggtgaccagcccgcgttcggagcactatgctgcgccgcagctgcacggctacgggcccatgaacgtgaacatggccgcgcatcacggcgccggcgccttcttccgctacatgcgccaacccatcaagcaagagctcatctgcaagtggatcgagcccgagcagctggccaaccccaaaaagtcgtgcaacaaaactttcagcaccatgcacgagctagttacgcacgtcaccgtggagcacgtaggtggcccggagcagagtaatcacatctgcttctgggaggagtgtccgcgcgagggcaagcccttcaaagccaaatacaaactggttaaccacatccgcgtgcacacgggcgagaagccctttccctgccccttccctggctgtggcaaggtcttcgcgcgctccgagaatttaaagatccacaaaaggacgcacacaggtacggaaacagctgtaggacccctacccattcccacttgggcctgggacccaaaccgaaagtcag

SEQ ID NO: 45 - DNA, 8000 bp, Homo sapiens
  BMP7 DNA region
ctgccatctaatttggagtgatgtcccagaaagctagacctgagaaagtcagggtcccacaaagcccaaagccagcctgactgattaagaggctcagacaagccggtctttgtcctttcaagatgaaagaaatggattgaaaggtgtatgtacagccagaggaggaaaaccacccagctgtggccttcccaaacaagtcccagcttgctcctgccgccatggggggagcaaatagcccgcaccaaagttcccagaggcaccctccgagcaacctggtctttctcttggggacagggagccccggggcctagcccacactgccggcccataagcactgggggcttttttgctcttggtttgcagggccttctgcagaagccagagattacaaatacctttttaatgattccagccaggctctgaaggcagccactgggacctgcacattcagtgggagtccggggtacccaacagaagggaagatgctataaatcccccaccagagaaagggccctggctaaccccaggcttcaggagggcgacagagctctgcgttcattatttatatgtatgtatttggaattttcctttagtttttggtatagttaataaatacacattgttttaaaaaaatcacaaatacagaagtgcagacgacgaaaaatcagaccccacctcccactccgtaagccccagctatgcccccgcctctctacccctccagtaaaatctctctccagttcctggtgtcttcctccaggaggacacgatgatatacaagcacacatgcagcccactcatctgcaccttgcttctttccacctcatacatcttggagatggttctaaatcaggtcatatgttgctgcttccttcttaactggctgcttagtataatttaaacttttttgaaggtattttggcatctaaacctcccaccccttgctcgctgaagcttggttcactagttggatttagttattattaagtacagacaatggcctcccgggaactccagggaccttatcaggagggggaagcatgaaggcggatctgcgcttcccccgcgcccccagtccacgccccaggtgcccagcgtggggcttcagtatcattatttccatgttgacacagcgcccgtgggcgtgagtttgctgtcttgacttggttcgggttttcgcagcaagttacacaagtagaatgaaagcggctcaggaaacagcgctatcggggtgtgttatctcggaaaccaaatatttgatcatcagtcccgcagcgcgctggcgctgccggggggttagtttgagcaagtctggagaatgtgcgcgcacatatggagggggagctccccggccctcgctgcagccccaggctcgcgtcgccggggtcgtcgcggctccagtcggaaaccgggacccccagccgcccccgcggcgtcagctctgagatcccgcgcaacccaatcgccaaaacctgtggtttgcgcgccacacgtggtaaatgctgaaaatagattctgccggcccgggacctaccactaaatacttaaaatagaagactagcggcagggcacagtggctcacgcctgtaatcccagcaccactttgggaggctgaggcgggaggatcacctgagatcgggagttcgagaccagcctgcctaacatggagaaaccctgtctctactaaaaatacaaaattagccaggcatggtggcgcatgcctgtaatcccagctactcgggaggctgaggcacgagaatcgcttgaacccgggaggcagaggttgcggtgagcagagatcgcgccattgcactccagactgggcgacaagagtgaaactccgtctcaaaaaaaaaaaaaaagaaaagaaaagaaaaaaaaagaaaaaagaaaaaaataaacaaagaaagaaaacaaagaaaattagcaatggcaccgtttcttccccagctccagccaagagagaatggggcatccggcggcccggagaggggccccaaggcgctcggagcgcgcggtacgctaaggcgggcgcaccacgaggggacaggtagggcttgacagggagggtaagggggtgcgacggccgcgatgccagagccaggcagggctgagaggggcccaggaagggcggcaaggcagcctcgccgggaagggccctttacatccaggtagcgcgagccgcgccggcccgggacagctcacatgaccgagccccgcgctttgaacggcccttgcctccagccccagagatgcggccggcggcgtaacatggaggggacttcggaggatccctccttcccagtaacccctacaggctcaccgaacccagcgcgcccgacctgggggcgcacacagcttgggagtcatagggctgtgggtgggagaccctcgggcaggcactgcgatttcagccaggagcggaaagctgggcgggctgcatagaagaagcggctccctccctcccagtctcaaagtgcccccgaaaggagacgcgtaccctcgcctgcccttactcaccgaggttgacgaagctcatgaccatgtcggcgtcggtgaggaaatggctatcttgcaggctggccagaggggggccctgggtactgaagacggccttgtaggggtaggagaagccctggccgccgggcccgccgccctcctccaccgccatggcgttgtacaggtccagcatgaacatgggtgccgagttgtgcttgccctggaggtgcgggcgcgggcggtggggcaagcccaaaatggagaggatctcgcgctgcatctcccgccgctcctggctgcggaggcgccggtggatgaagctcgagtgcacctcgttgtccaggctgaagtcggccagggcggagcgcagcaggaacaggggtgcccagagcgccacgaagctgtgcggcgccgcagctcgcagtgagcgcacgtgcatcgcgccggctctacgcgctacccgggctccgggctccgggcccgcaccgccccaggtggcagagggggcaggcggccgtccgcgccgctcggtcacttgctgcagacgggccccgctgcgcccgcgccagacatggcccctccccggccggccgcgctctgcccggacccccgccccctgctcggtgctggccccgggcccctcgccccgcactcgcccgggcgcaccgcagggcttggaaagcagccccggcgaacgaaaaggcgagtgaggaggcgggcgcgggtgggaggagcagccagcaagcctaggagtccagagagccaacgccggggaggcagcgaggaggcaggcgggcgggcgagcgctccttcttcccgctcctctggcgctctcgccctcgggcggcggcaacccaccctcttccaacctccggcgcggatagcaggggccccggggcgctctcccagccttgtgcgccctggatcgcacaagaggccgcctcgggagcgccagagtggtacgcgctggggcctgggcggaggaggaggaggaggaggacgggcttcgaggggcccctcccgcgccggcggcctcccacctgccctcggccccgccctccccccagctgtgcccagtgggttcattcattccctctagagcgctcgacaaatgtttactgagtgcccacggggtgcctagagcctggcgggacgtcgcggggatgcggctggaggaatcggagccggccctgccctctcggcactcgcggtccggaccaggaaggcggcccgagcaggacgggtgggctgcggaccgcggtgggaaaagttgggggagcgcggaggtggcgcggaaatagaaggcttcctggaagcggtgacccttgagccggtcttgcccctcagtccctgtatccttttaagccagccacgctgacccctccgcgcctccctgaagccgttgccagcctgtctctgcgctcgccattccctccgcctggacctctccccactcaccacctcccgcccccatcagacgctgctttctcctcccagagacctccaagacctgccaagctgaggttgtttcctcgcctcgcccctcgcacgtttgttttctttcctgtagtttctcttgctgtctggaattgctgtttgagttactccgtgccttctttattgccttcttacccccgctagacacccagagcactggggcccggatcagcgatcttctccgcacctctggtacccgcagtgccttgaacccagcgcctggtagccactgtagctgcgcagtcacgatttgttgagtgaataagtgaactgcgtgtgagctgggggggttcttcccacctcctcctcttcggggccctgtatctttccagccacccctcagcctgagaccagacaccccagccccaccactagggcttcccatcagccctcctcgtggcagtgcctggggagccagccgctggtcttcacatcctcttcccctccaagggtctgtgcctgagacctccccatcggcctcctctcctgtctctcctcatccattcatccctcaatcaataccgagccatgcacgctgtcacagggagtccccagaccactcagaacccaccggtccctctgccggtgtgggatctcaagggaattactgcacccagagtggcagtgagggcagcggggtaaaagttcatcctgtctccttcccctcctggggctgggtgagagagtgggtccccagagggcctttgggatgaactgggaagggctgaggttggggaagcgggggcgcttgtctgggaggggctctggctaagcctcactttggggctgtccttcctggagcaggagagctggggaagagaattatggtcttctacagttttctggagcagaagtagtttggccaagtggccctctgggtctcccttggggacttttggactagatggcagcctgaggggccacatgggtggagaaaggggaggccagaggccaggcaaggagcagaaaaatgccagcctgcaggtcctggccatttacacacaggccccaggctgtgggatggaacaggtcttgagccccatgccagggaggggcctctacccacccaccaggcaaggccctcaaaccacccccaccacctgctcttcatccattcactccttccttcaacagacaagcatggagcacaggatatcaggaggcgagctgtggggcctgcaacctggacagtcgtacagggccccccagctcttaagggccccacacttggtttcctactctgaggtcaccatatgtggttctgaatcacctatgaacaaagtaccccctgccccgtactaattttgcacctggcccagatattatgtggccagtcccagggccaagcaccatgctgggcaggaagacatggccatgagcgaacccacatggcagactccatcaaggagccggcctccaggccagggaagcaaagcaagagccttaaataagattcagggactgaaggagaggcagatcttgtctatcactctctctggaaacaattcagaagccaggcagaggaggacatgcaggagggagaaggaaaggtgggcatcctggcaagcaggaatcctaccccagggagactggggaaattgcaggagtgccacagtcgtccagggtccatctgagagtgtacaactggagagcctgggtcacttcagcctgcaataagtgacaggcagtgggggcgggggcgcagataaacagggtgaggtgatggagggtgggaaggggagtttttggaggagatgccactgacctgagtcctgaaagacaagaaagagacaggcttttccagggaaaggcatcccaggtggaggaacagcaagtgcttgagccctgcagagcagggaacagaaaccaggctcggtccccagccctgcacacgccctccagcgctggcttgctagggacaggctatgccacctccagtgccagtagagaaaaaggaggtcattttcttccaaagtctgaaataataaggagccaaagcagccctgttcttcagtagtagtctctgctttttatctactctcaaatcttttggagggatcttcacaagcaggtgaggctgggggcaggggcagcaggtcttggtgctacaatttacatatgaaacaactgatccgtcagtgtctaagcaaggattctagtttacccttctttgtgcatctcacctgagcctattctgagcctggcacacagcaggtgctcaattagtacctgttgaatgaatagtaataagcacagccaaaacctaaagagaccggttgactgagggctgactgtgcaggcgccaagctaaggcatctcccagatccctcaccccagcaacccttcctctgagacgccatcttttctcatgcaagaggaagagacaacatgctcttcatatccaagacctcaagagataaggtttcctctgaagtcccacaaacccccatttagccttatcttttctgtgatccatgcttggtgggggggtctttcttttccctgtttcctccccgagaggcatccccaagactgcaccaggccctcagtgaatgccgaatgaatgaatgaatgaatgaatgaatgaatgaatgaaaaccggcaagcaagaaatatctggctgggagtgcatccgatgccagtttgggagctgagcccaccaccagaaaaggctttgagattgccgagggcaagtcttgtgtggaggaggtagcctgggcactggagcctgcccagccgcattcacactccagctctgtctcttccagctgtgcaccatgggcaagggacttaacctctctacacctcagttcccccatctgtaaaatggggtgactgatagcgcctgtcttctgggatcgctgtgaggttgcagaacttaattcatggaaagggctttgcacagtgcctggaacacagagaaaacccttgataaatgttattgtgttaccatctggatgaaaaggaaaatatggtaacaggaaagcctcctgagttacaaaataacaacaaaacttttcatgatggtctgctttttggccaagcagagaaaagcagatgtatcctgactctgagagcaagagcaaaagtaccagagcaaaagagagagtgaagtggaagggaacatgagagatgaaaaggtcatcactgcattccaatcctcacattgtgcctaggccccctgcaggccagctggggcccagggacttccctgggtcctatctcatttccccagcagctagcccccaacccacacacccccagttgtggttcccctggccagggcacccagcactgggctccaggaccgtaatctgatctgccccagtctgctccacacctggggccgcctttgcctggatgaaagacggcctggaattgtaaggggtgttattgtgtcgggtggcccggtcccagtgtccgcgcccctcgcctgctggcctcccagtgctgggccaggagttttcatttcctacatcatcagtcacatcttgctgaagtgattttgaccaagtcagcatgtctctctaaggcagtaaaaagaagaaaagaattctgctgacacacacattgttcagcgaggtggggacctcatcttcatccatctgccctagaggaggctcagctggcctggaagctccctggggagcacagccaaggaagccagtgggctgctctctctctgtccacaccacctggcaaaggccagatgacctggacctccaaccccaacaaggaatagagggccaagagctgcctcctgcacaggcttgaaaatcgttgtgtgcagaggtggaccctgcagaggaaattattagagcacacctgtggtgaaattttgtttcccacgcagatctctcattgcacgggaggcttggactcctcacgcttgcccactctgagattttctttgcacaactttaaaagcagcagaataagagattaaacgtcttccatcagtcttaagtacacatggcagtggccacagttccaaatggaaaata

SEQ ID NO: 46 - DNA, 8000 bp, Homo sapiens
  IRX3 DNA region
acacctcacttttatgactaatgaaaggggtggagtggaatactgctgggagtttaagaagcgaaatgggagattctgacatgtaattttaatagtaaaatgaccaggttctgaggatcaagcctgtaatcccagtgctttgggaggctgaggtgggaggattgcttgaggccaggagttcaagatcagcctgagcaacatagagagaccctgtctctaaaaaataaaaaaaatagctgggcgtggtggtgtgtgcctgtagttccggtgaatagaaggctgacacgggaggatcatttgagcccagaagtttgaagttgcagtgagctatgattgtaccagctggggcaacaggctgggcaatactccagcctgggcagcagagtgagatcctgtctctaaattaaagaaaaaaagagtaagattgcttctgcctacctacctcgtgtcagccctggacatgcttcctctcagcctaaaaactgcccccttcgccctcattatttccattttgcagatgaggagataagggctcagagaagtgaattgtctgcctcgggtttcacagggggtaagtgggagtagagtcggtgccacagctaagcttgtgaggcgtgagctgttgaggtgtcggagtgctctccttgtaaagtcattcaatgttccggcctcaggtccaagtccgcgaggatgggcgggctccacgtctgggggcgctgtgagcagggccagagcgaaacctcagtctgagtttacgaactgggaaggagcctgaaccccagaaacaccgttaacaaagagggcgaacgctagggagccgaggagacgtctccttggcctctggggaaggggctggaggagcgagccagctgagaagcggctttgccgggtgtccactagcggttcgccagccctctgtgcgcccgcagagtgacgcagaggccgacacagtcccctccccactcgcccctggggcgccgccgcaccttgatcacccccttcgcggagggaactcccaaggcgcggcgctggccgctgggtccgcatggcccagtcctgtcgggctccgctctgctgccgccgagcttcgaaggtgtgcagtgttgagccgtgtcttgttccgtggcttcgaggggtgtgcgcgcttcctatacctccccggaacgcgcgagatagtcacacgcgccactttgagggtcaaacaccccgcatctggccacactgtacctttactaacgtggggagggggcaaaaaaatgtgtctctccttcttaccccgaggtgtcactcgcccaaacactccctacaacttcttcaagtcaaacttgggcaaggttggctggctgactgcgagaggaaaaagagggcgcggagggggcgcggcgcgcggccgggtgtagaggccacggaggcgaggcgccgagcgtcccctttgtcctgtagagggagctccagccccaaatttccctgctgcctcccccggcccgcccaccccaggcccgcctggagccggaatcccggctggaaaggtgcggcggtctgacacccccgcaacccccgcgccggggccttagcaggatgcctcttctagggcacgtgggaaaacccaccagggtgccaagacgcacagatgctcccaggacccgggctccccaactctcaactagagctgggcagtcacacatcatctagaggattatgtacccctaggcgaccctcttctccatatccgcctccaccgtcaccccacaccctggcatctataaagagggaaggggggcaaccgggttggaaggaggatggaatctgggcttgcacagcctcttataggtaacagtgagaaagcctcaactgtgtcgatcaaagaatggggacgaagggagtgatgagaacacgaccccgccacccgcgcaggctaaccccgttgtgtccagacccttctctaaatctcttggcaaggtcaccagagaaggatccaagcccccgcggttgctcccgtagacaagcgggtcgggcccagattcccggctctcagcaaagaagcttgccggggtctcccacctgtctcctgctcccgttttctcggccgccccaggtacttttccagccacctctcctgctgcggcgcccccagccccgccacttctgcgcctcctcagctattgctgtcctttgactactcggtcccgattcgtctctcgtcctttcccgcgaattcttcaaacaaccgaggggagaaaaaacccaaaaaacaaaaaaccaccaaacagatccgtcgagggggcagacaggcgggttatagtcaaagtggcagcagctcatttatttagccaaaaatagatactttctcgtacaatttggttcacacatatgtacatttctctgtatatatacacacacacaaaggcagacacgtttattctcactgaaactccaccccccaaaatcaaccggacaaacaaacctcacagcgaatgcggggtgccacagaagcgacttggggagggctgaggaggactggttttatttctttttcttactttctttgtttagttttgcttttaggtatttatacacggacatgcttacaagttgtaactatacagagcgattttttttatacaattattacaacgattaaaaaaagttttttttgtttttttgttttttttaaagaactaggatgaggagagagccgataagaccagggcggcgtccagatggttctggggcctggaagagagagacagtagtagcaaaagaggtgagattcaggagcgcagaggctgcctagggcggggagatcctacttggattggggccgatcgtcaggaaggtgtcgcaatgtaaaattatgtccagttgtagatgtgtgtgttggggtttaactgtagggtgggcacgaggcgtcaggacccgaggtctggggctggaaagaggtgggcgtcagagaaccgccacccgccccaggggcctgtgggcccagccacgcaaggcttcccctagaaggtacaagcgctgtaccctccggccgcgcctggggtgcctgggctggacctggagagctgtcgcagggccgacaggggcgactgaaaaagtgactttcgtccccttttacaaaatgcgtcccagcagggttcccccctaccccggggcaaaaggccgtgcgtggtgctcggcgtcctctcctttccccgccagccagtccccctggcccctcaacctcggggtttcttaccgcctgggcacgggctggaaagctgtcttgagtaactttttctccacttccaaggcactacagcgatctaagggaagcgggggaagaaaaaggagggcctttagagcgctcggtgccggcgcccagggccgcagaaagcaggagtggagagggacgcgcgccctgcgccccccgggccctgcccctcccggccagctccggcactacccgcagagcccgcccccgctccgcgcccagcgctcagcagtcacctgttccgccttcgggctccgctggccgagcgaaggcggcggcggcagccgggtggcccgcggctccgggaagtcccagcaggtgcggaggggcagagcccagcagggagagcgggtgcaggcgggggccgggcggtgggcctggaaacggccggttggtccaagccgggaacttgcccagcggcgctgagaccagtctgtgagcggcggcggcggcggcggccggagagagctgcagggcggaaggcgcgaccgctgcccccggtggagacccccccgcgccgggaggcgagcggcgcgggttgtccgggcttgtggcagtctccgcgagggaccagatcttgggcttctgcagggcggaggcgggggctggtgcgggagcgcaggggtccaggctcacggggggcgacggcagagacggcgaggccacggccactggtggtggcgctggagccagactcaggaccggtagtggccggtcctctaagccctcagagctatcttccgagtcgctatttttggagtccgaaatgggtcccaggcctaggtcgccatccctgcgcgccgccccagccagggacagctcaggctcggtggccgcgccgtctaagttctccaaatcgatctcctcgtcctcgtcgtcgtcagccaggccctcgccccccgtgtcctcctcctcccccccgagctcctcctcctccagctctagctcgcgtttgccgtcctcctcgtcctcctcttcgtcttcctcctcgcgctcgctcccataagcgtttccctcctcgtcagtgcggctgcgaggcgcccaagtcatcttattctccttcttgaggcgccggcgcgcgttggcgaaccaggtggacacctgggtgagggtcatcttggtgatgatggccagcatgatcttctcgcccttggtggggtaggggttcttgcggtgctcgttgagccaggccttcagcgtgctggtgctctccctggtggcgttcttgggacgggacgggtccccgaactggtactggccatacgggtagaaggcggggtgcgggtgcggaaacgcggcagccgcggccggatgctgcaccccggggctgtccttcagctcatactgcgcgccctgtacgcacatggagaaggaagggacacgcgcggagggagcgcgagtgagccccagccatcgctgcctcccccctcctggcctgcacccctctagtccggcccccgcgcgctcagctcgccctgcgccccagcgccaacccctccttccctggctccgcgggctcttaccagctgcgggaagatgggcagctccgcggcgtagggcaggaaggcgccgtagccttgggcggcggcggccgcagcggccgcggcgtagggcgccccgtacacggacgagagcacgttggacagggaccccgaggcgttcagctccgaggctccggcacccaggccgccccgggcccccgcgctgccgccgctgccgccagcggcccccgggcgctcggacgggtaaagcgggcggatgtattggtatcccagctgggggaaggacatggtggcccgcggggcacggacggagaggggggccgacccccgggccgcccagctcagcgccgcccgcgggctccggcgcgcatcgggggctgggccgggcttggggccgcgctgccgcccgcgctgcgctgtgctccgcgttcgcctattgatctgctccgcggcggcgacggcggcggcgagggcggcggcgaggagccaggtcaggtccgaacagattggcggagattcccggggctccgggctctgattgacatttctacggggcgcaagcccctcctctctgcgccgcgctccctcctctcggccgccggagctgcctctgcccgctcctcgctcctcactgccctcctctccccagcagtgtcgcgcctcgcttccttcgccctcctctagttttcactccccctcctcgcccttcctctcccctcccctctccgcactcctcttccccccaggatcgcttccgctgcttcgggctcctgcactgccctggacgctatgaaactctcctttctcctctccccttctctttccctttctcacgagggcttttgctctccggtccgttcccttcacttcttcctttctcactacttctattttgtaaatctctctctttactcacacgatactccccgcgacctttccagcctctgcgccctgggctaagtaaggcagccaaaagttgtggacacttagggtgccgcgcggggggtgtgtgtcggtgtctgtctgtccatccccgcccttccaccccctccctcgcctttccccaaatctcgggtctgacgtcgcgccctcttattcgacttttccccgcgtctcttccccgagtcgccaatcgcctgggcgccttacgggcgaggccgcccacccccgcggatcccgcgcgcggggcggggcggggcaggggcgggagccggagcgccgcgagtccctgcccgccccctcctgctcgcgccgggcgggggagcgagaggcgcggggactgggtgagtgcagggtcagctcgcccctcccgcctcccgcccctccgagcctgtccccaggctgaggcgcgggacctgggccgaggctggctccgggagctcccctgtgcccgggccccttttacagctcgcacctctcgactcccggccactttcagcagcttagggagggagctagcagccggcgtgggggtggggagcgagggagtgcgtcctaacagagtctccagaggccacctcgctgccttgcgggggcgaggcagcccccaacccagcaggaccaaacccagagattagccacttaacgccgcccccgggtccgtgtgcgaatcccttactaaaaatggcgggctggctccgggggctgaagggtggcagcccgggggcactggaagggtagggagccgccacaaatcggtgaagcaggcccgcgccccaggcggctgggaatgggggaggagcagggcgccgggggacggaggccctaggaacttttggagagctcttcgagaagtttctgtattggcgtttttgtctgtggagaaccttccttgcgcgacccctaccccttctcccggtcccggcgctaacctcttttctctctgcaaggatcctcccaccgcctccggtggggatggaaagcaagagttttggtttaatgatgggatatttcctaaatcaaacttgaaatctcaggatcgggcgcccaccttcacggattagggcgtgcagtcgatagcggcattgaaattcccagcattgccgcggctgggggctcagagctttaggccgcggcggcgacccacaagccggccaggcctgcagtgggcacgccccggagacaaaacgggggtggtgactctgccgtggagtggccagccccagctcggaggcggaaaccgtcaccttgcatggatggatccaggaatcgaattcgaattccaccagagtggcgggatcagaggagatcagagggtcttggccccggctgtctctgatcacagctctggggagcccagctctggaatgaaaaattcttccacctccacggcagacacccagttttgatcccacccccaccgcccacctcccacttcccacctcccacctctttccgtgggtcttggcacaagcccacggccctagtaggtgtgaacgccaaaaaccttgcgcgggcgggatgggggcggggaggaaacaagggaaagtttggggaggaccaggttttgggggcaatgagggaaagcggggtattcccagactgcatctgagtagtttccctatcaccttctcttccgcctctgcctggtgctggggagcctggtgccttctcaggagcctcgccggaggaggcgtcatcaggggccgccctggctttgaaatatctcttcgacgcctgatttcgtttcccgttaaacctcacagagagtggcatagggaatgttgggctcactttcacgcgaagaaacttggggttacaaaattgagagtaacgttattttccgggatgtttcctaggtgtcagacctcgagcctcgacggccctgaagtcatagtgtttgtacaaagtggtggcaccagaccaggaggagctgagagcgagattccgaggtggggggaatggggctgcgctgtaagtgaccgagtgagactcccgatatttaatttagcttttttcagaaggaaaagaaaaaacaataatgagcctcggagatgctccttctgcattaaccagctatcgacctgtctgcgtcggactgcgggcggcgagaaagaggacagggaagggccaggcggctcttagggactcagtcacgccggcgggtggggggggggggcgcccacagcccaggggatgaacctccagaccgagacgtgcacccaagaacctcgcatattagatccctccaggctccccaggcctcgagcagaagcggcctcctggctaccctactttccaattga

SEQ ID NO: 47 - DNA, 8000 bp, Homo sapiens
  WNT6 DNA region
attcctgtaaggcctttcttggtctgatttcccccaaatccctccagcctaatctgccaccaggctctgctgttcactctcattccaacctctttttgcccactcccccactctccctccatcactcccctccactctccaccgcacccgcctcctcctccctgcaggacttttggaaaggctgctccttctgcctgaaatgctcttccctccctgtgtctcccagttaacgccaacttatttattttacatttcagctagagtgttccctccttagagaagccctccccgcccccatgacctggtcaatcctcatcccacagcctcttacaggcccctcccttcttttccccagcacggtcaaggttgttggctttccttttgcttgagagatgatgtgattaaagactgtcagctccatgtgggtaggagttatgtccgcttttgctcaccatttcccccctcccccgctacccagctgggagcctggcacatcagtgtccgattacaaatttgctgactgctgcccagggagagagacagcagacaagacatcaatagggaggctggctccagagacctggatttaggagccccgtttcccgcagaagtgagagctgaagcagcagggacaggtggtgtcccaggagcagaatggagcagtgagacgagaggagggcagaggctcttccctagaacgcttgagaccaggccaggaccctttgcccatcccacgtcagtaccacaggagccagcctgggccaggatggctggagaggctcctggggccaaccactcccacttgggcccatggggctaagctaagtggaaacaaatgttccctggctttcccccagcccagttgtggtgggcgagcagcagcccctccttccctagcccgtgctctcctgccccattcaagctccccctgggttcttgctccccggactggtgggggcatggataggcttgaaagaggtggaagatgggaggcaggtgggctctgcaggaagctggggcctggtgtaggggattctcttatccctgccttgactaaagctgggaacttgctctggaaaagcacactttcctgacttcaggtgggctgcgggcgtgggagtagagctgtctgtgatgcagaaggaagaggaaaatgagcctctctcctcgctctcttttctttttctatctttctccatctctctaaggggctggcctttcctctctctgttctccagggacccagaaacccagggttcttaggaattctcaaggcatgtcacctccctgaaggaataggagttcctggggacagagagagggaaggacagataggagttctggggacagaaggagggaagtagggagggaaagagggaggttcttgggaggggaagggtcacaggcccaaattcctaggctcctgcagccttagccattccgagaaacgacagagaaagtgatttagagacagacacaaatagagaccgaattatgaacatgggagagatgtaaaaaggcacagaaagacagtgagagagaatccgccagagacagagggctttgctttcgcccgcagcgattctgatgttatctatttccagctgcctctcccccaccactccactgcccccactctgcccctccttcctcctcaacctcaagtctctccttccctgtctctctttcgcacagggcctagacatagtatgagtgggttcccaggatttaaggcggagcgatgaaggaaggtggggtgggcagggcctagaggatagggaggggggtcagactgtcacctccccattcaggccacccatttccgggctgctgtcttcccactccagtctcttgtgaatgttcttctgcatgcatatctctgcagaggtgatgtgtgtgtgtgtgtgtgtgtgtgtgtgtgtgtgtatgtgaagtgtttcagcctgagccagcccatctgtgtgtccgtccatacctgtgggtaaaaaatcacagcaccgtctgggggagggaagatgtcctgtggtttctgtggggttatctggaggcctgtgcccagtgattggtgtgtgtgagggggcatgtatgtcacatatcttgatctatctttgtgcaccatggtctgttacctcctgccaccccctctccagctcctgctcctttgtaagtcttctaaggtgatagtggtgggttgaaggctcaggaacatctgaattcatctctttggaggtttgggggcaaaacaacagaagtcatcactctggctctgcccagatgccagccagccccttgatggggatgcggagagggcaaaggagagtgagcaaaagggtgctgttggggcagaagccctaggaattgggactccaggagtcctgcctgtcacagattccagcccacctccccccgaccccccaaccctataatgctctgcaggcactactgggccaactgattctggaatgggactgagtgcaggagggacaagggcttggggagtccagagatggcaaaagattctgcaagatctggctagagtgagtgctgcaggaggggtggggccagctgtgtatgtttgcaggaaaggtggggggtgggggcagaaaggccgcctgcgggggagaaggcttcttggcaaacgccccagtttcctgagctgatccctgctccctgccactgctcccaattctgttgtttcaataacatcatttttatccaccccacagagagactgtgtcttaggaaggggaggcgaggggcagggctagggcatgggggaggggtggtaatggagagaggctgggtctggaggcagcagggactagatttggggcgctccctcctcaggcagaggcagggcggtctcagagcccaggtcccctgatgacaaagacaaatacattaaaattcttgcctcggagaagaatgcaggatagggagagcatttcataatctgcaataacagtagcagggtgggagcagggctgggcagggggttcggaacccagacagtggctcccaggaggctggccactggccacctgtagcctccagggacccccagagccctctagctgcctatttggtaccacatggctggagtgtgcacccaaactgggtatgtctttgaatgtgtagtgtccagagttggcattgacctttaggaactgtccagattggcattgccccgtagaaacagcaactgtgtgcaaagttactaggtgcaggggctgtgcgtggcctttggatactgttaaaggctaaaatgggagagggcagtgcttagaccagaccctctctctatgactcttgccactgccccctgcccccttccccttctttagtgtgtcctagccctttcaccagaactctctgtatccagtgcaagggattgagctgatgcagcctggacaccagggccctgacatggagcttgcttgccggcaagtccacacttctccagttctctcacctctgtctgctcattccttcccccatcagcctcctcccctggggtcacatcccttggccccttggcctcatcttgatctacttgtcccccctctctgggtgactcaggatcttgtcttctctctctgctcaccaaggttgtttggaagtgacctcagtcatcactctcttggaagcgacctcagtccccacattcaggtccccctctctaccaacaaccaggagctttcttctgggcgcctgacgtttctgccatagggactcgatctgtcctccccactccaggacagctttaaaggttgatggttgctcttgatggtatggcagggagaatgtcccacaccacctgtcccttggcaccccggggcacgtcggggacgggaggggcaggggctgctgcccactgccagccgaatggcatgatgaatggagctgtccctcttccccacggagcctttgatctccccctcgtggggagggggccaggggcatcaaagacatttttgtggcattagaaaatctgataaacgccatctcacctgcagctggctctcccaccccttctacccgggccaggttgggggaggaactcgaggcagggactcttggacacgcgaggccgggctaaagcgaattctgacccctcgatctcttgaaagtctgcgacctccattctcccgctcgcttacaagtgccagcgcttgcgcgcacgccctggagcggctactcttgccctctcatttcccccacctccccgccctagaacgcgggcttgagaggtgggatggctgggggagcgagagcggaggcccagggcttcctcccttcttcaaggagaggctgacgaaggggcatgaggcatcgaacagggacacggaaagagatgacccctccagcatccgcctgtcgggcggtccagtttgacaaccctcgccgaagcaccccgcgccccaggccaacttcctctcttgcctccgcgccagcatctccctccgcaccgccccccgccccaaactccctcattcctgggaagttttctctctttacagtcaacaaacctgtcaaacgtctcccttccagcccgtcccccattcctcagctcctctttctcccccctcacccctctctactcttcttcccagcccttccttgcccccgactccaggcggccgcgagcggcggggggctccaagtccgagagaggcgaggcgagggggcgaagcgggcgggtcccacagcccgaagcgcgctggggccccgccccggcaccccgctcccgccctccgctgcgctccgcgccagccgccccggggcgcacggctcgctcccgagagtcccgctgcctgactgtcgcaactgccacctccctccggccccctgccccccgcccccgcctcggccccgctcccctcccacctccgcccccggctctgatttcttctcccgagcgagctccgcaggagacacaggcgctggctgccccgtccgctctccgcctccgccgcgccctcctcgcccgggatgggcccccccgccgccgccggatccctcgcctcccggccgccgccgttgcgctcgccgcgctcgcactgaagcccgggccctcgcgcgccgcggttcgccccgcagcctcgccccctgcccacccgggcggccgtagggcggtcacgatgctgccgcccttaccctcccgcctcgggctgctgctgctgctgctcctgtgcccggcgcacgtcggcggactgtggtggtgagtccggctgtcctggcgcggctctggaccctggagggtggggaccccgggacccaggacagactggggtgttccgcagcctcccggctctacctgctctccgagcccagctttctggcctcggccgggggcgtttctgaactccacttcgacgttcctaaccctcgggaacattctattgtgtttcctcctcccgggttctctcccaccccccgccaagttatgcgctcacccagctcaccaaggaacaggcgagtgggtaggaaagatctttcgggaagacacatatccgaggaaaagaatgtgtgaggagggccctggattcagggtatggggctggatttggagaagacaggaacaaggtgggaagccacaaggcagagagggccacacagctgccccttccccgccctgctttgcctaccaacccaccggcttctctctgggctcctgaggagtgggccttagtgacgacagagggaatataaaggggaaagtgagtcaggaccctggttttctctacctcgtcctccccaagcaaggaaaaggaggagcggaaggaaccggacccaggcgcccggcccttccagacctgggggtcatgaatgaaaaaatatgtgcagcccgggagatacgggaccgggaggggtcctgcgggaagggagaggggcctggccagccaagggaagggggcgtcttcccaggcttggccctagctagaagggacacctaggcagcgaaggttgcgggagcttcgagccttgggggtgggggtggggccggggcggggaaggagggtgcccgaccgggacggctcagagatccagatccaaaggacttttttccccctaggtctccagagccgcaaaccctcgcccggaatagctcagtgtttagagaggagagacttggacaataacgctgacgggttagccaatcctaagaaggcagggtcgtgtctatttgcatattgccatgtgattggctggatgccgaccctccgctcctccctcgccccccagatttggaagagaggcagatccccgtgctctggagagatagggagtaggatgcccacacagggagtggcgggcagcaatccctactccgtgatgcggagagccgtccgacgctagtgtcacggaaagggcgaaccgaggacgacccaggtcctatctagggaacatatgccctttgatccggcttagtataatgcagtggcagctttgaagcagatagatctaagatctaaacattatgtttgtgatcttgggctagtcacttcctttccctacctctcagtcttaactggaaataggataacagtctccctacctggcaggcttgctgggaaggttaggtgggaggacgtgtagaataataggaccttaggaggtactcagtatgtacagcgcccccctcacaacccagcttctcctggccagggtccccaggagtgaaaaaggggattcatttacatttaaaatccagagttccaaggaaaatgttgtaaaggaatctcttttccccataaacgcctaactttgccctttttcaaggtgtggggatggggtccctccctgatggccattgtcttccttcccctccctccccctctgtctaaatcattcttatccttacggttaaaataccatttcctccctgaagctgcccccttgtcctccttccattgccttctctgcgctcccactgtatcacatacattgggcaactatgctggttactgcatttggctgacaacagtcctttaggtctctcttccctgctcctggaaggcagataccttgtctttaggagccctgaggcctaggagtgtgttggtgctggagatgggcttgcagagtgtcattggactggctgattcacctttctgggctctgtatctgtggggcttcctttgttcaggaaggactgctgggtggcagccaagccaaaggagagtggcctggtccctgagagactgaccctgaagctgggaagaagggagtgggggaggaggtgacacgggcaggtgtgggagaacacagaggccttcatggccacagctgcagggcgtgccgggtgaggagtgaggggtcccagtgataacgtgtctgactgccttgctcagatgtgcctctgagggaatgagcctctcctccctgccttggccccttcactctcctttgtctattcattttgtttttctttctttatttctatttttatttatttatttatttagagcagagtctcgttctgtcgcccaggctggagtgcagtggtgcgatcttggctcatggcaacctctgtctccccggttcaagtgattctcctgcttcagcttcccaagtagctgggactacaggcacccaccaccatgcctggctagtttttgaatttttagtagaggtttcaccatgtttagtagaggtttcaccaggtttcaacaaggtttcaccgtgttggtcaggctggtctcaaactcctgacctcaagtgatctgcccgcctcggcctcccaaagtgctgggattacaggcccagccattaccatggcattaccatgcccagcctattttactttttattttttatttttttgagacggagtctcgttgtgtcgcccaggctggagtgcagtggtgcaatattggctcactacaacctccacctcccggctcaaatgattctcctgcctcagcctcctgagtaactgggattagagttgcccaccaccatgcccagctaaattttgtatgtttagtagagacgag

SEQ ID NO: 48 - DNA, 8000 bp, Homo sapiens
  WNT10A DNA region
ttgtcaaacatatgtttagtaaatgatagttactgtgagaatggggacagacatatgatgctttgcagatattgggcaggccctgattgcaaatgaagatacctgacttttagctgaggccaaacagaaataatgaatggcttttataccagggaagtccagaggttagtctgaattcaggtacagctgaacccaggggctcacgtggtgtcacaggcttctttctcatctccttccctctccctcgcctctgcatctattgtttctgcttatctctgcctctcttggcatactgtcctcattatctcctcctgcagactttctccatgacagctctatatgcagtcttagatccaaaagcagaagaatgactttcccagttgtctccaacagaagtcccaaggaagaccttgcttagttcagcttggtcatttgcctattcctgaaccaataatggtgtctagggagatgtactaccatgcctgggattgacagcctctctagaaccacaggggggtggggcaggcacagggagcaaagcacagtccttcaaatgaaacagaaggaaagggagctcgttctggatgtgacagaccctcaccgtggcttccaggacccctgtccttcccccgcaatgcccctgcattcactcggtgacctagcctgcttcaatcctcaaggctcatggtggggaggggtgtcaggctgcagtgggaaggttgaggcaccagggttttcctttctgcctcctcttggctcccttgggtcaaatgtctgggcctcagggaagtcatggagaatttggacttagaactccttgaccaaaggaaactctgttaacttaccaaaggagacagtggaaatacattatggtccacatagaggagaaatgaagctccctgagggcaaggtttatattcacagatctgccttccccaaagctctgctcttatcttcaaacaaacaaaaaaaccaaacaagcccaaaacccaaactcctgcacttctctcagagaccaccagagggcatgcggagcccagcttctctattaacagggagcgacagacaggacctgcaggacatctgggaaactcagagagtggtgggcaggagaggataggactgaaaggaagcaaggagacctacagacagcttgggtcaagcttctagggttccagactgtctgggacgtctgggctggcccagacctagggctgggatgccagcctcccagactctaccactgctgggtcctgcagggggtgagatagggtggaagatgctggacatcttcctccacaatgccagggagggaagctggggaagtggcatgaactggcaccattctaggcaggttaaaaggaattcagaggaagttgtatggtacaatgaaaaatgccttgtttgctagcttgaccttggtcaagtcagttaacctatctgagcacttcctatcacctaataggggtccctgattgctttttagcacattgtgagaatgcacttccatctttaaactgctggccacatgttactggtccatcttctgttaccaagccctgaaggcgtctgggcactgccttctatgaagggtgggagtgattaccagaaggcaccattgggacttctagccttatcttctttgagcccagagatagatctgtgctgttgccctgctgcctgggaaggggccggaagtagagttcccaccaccacccacccctgctcatccagctgctcagcctcaggcagtctgagccatcaaagtcccctgctggctgaatggcaggggacatgtggaactggggagatgggggaagtgcaaagaggcaccccagatgtgtgtgtgaccacatcaggaacaggcggaacacaggcagatgggtgaggaggccgagtggaaggcctaggatcagcctgaggccacgggctcggggaacaattcccttccctttcaatgaccaaacacattgggagtttggggagactgcagcagcaagccattcccccttaccttgctgagggttggtggcctctgaggcctggacaatggaatgaagagcctccttattggtctgtggctccttggcgttcttcttcagcaagacaaacttctccagctgggcaccaggactgggctggggctggactgggctaggctggggctgtgagggtcccacctgttctcacccccatgcttccccggcccttcctccctgtgaccttctccagagctgccacagctggaaccattctccaagctgcctttcatccccagtgctcccccgccccccagtggcccctgtaattgtggtcttaggcggcggctgcgggggctggggctgggctgagggccagggcccagggcagggccagggtggggactagggggtgctgataggggcttggccatggtgtgggtcatttggggctgggaccccaggggcagacagtctctgggcctcacctacatacatgccaaggctctggaccaacacagactctcccctcccttcactgagaagtgctctgacttgggcactcctagaccctcctgacaccagggcagagccacgcctgagtgcaagcctcagtatcactatttgcctccggaggcttttttgtttttttttaagaaagggttttctcggtctcctttgcacctctgtatttcacagacagtggaggtggggaagaaaagagaccccctcatcagagagatgccaaaggaaactaaatcagctcctcaaccaggtcagcagcgtttcccaaaaggagtccccaccagaatcactggggatgctagctagaaagacagacacccaggcctttccctggaaaggctgattcaacttcatggggcctggaatccgcattcccacaatggcccaccccaggggacacttgtgctgcatcaagcgtgggaaatgctagtgtgagcgcgtgatggggaataaggacatctgtaagctgattatgagtcccttccctgtgtcacgttcctaattctttctgggcctcagtttccccaacagccaataagaatatgactctccagcttgctggtgtcctctctcatcctgggctggtgcctgtctcctgtcttgagtctggtcccacccttacccttaccgcttacccttcccaaatgctagtcccaagctgtccccgtttctgcagcatggtagggagagtggggagggcagggggcagttggggtcaaataaggctctagctgacgcagtctgcttgctgcccctggctgttttcctgcagtccagccagactgctccccccccactgccccacccccacccccagcagcctcaagcccttaattcctgacagctgcggtggctgccggcagagatgagggggaaaggaggggggagtatgccgtcagcctgctgttgctcaggccgcccccaccacctgacagtctggggacaaggagggccccaccccctccttcagttccccagggatgggcaattccaggaatgggccttttttgtctctgagcccagaggctcccatagttttgttcctaagccagcaggggctctcttcagagagaggggcatgtttgtgtaaaggcttgtgcccattttacagaggaggatgccgagggcacagggcaggagcacgatccactatcagggcccaggctagcaccagaaacagacggcagagctgcctttcccctgccctctggctctcaggcccttgcttgggcccagcacacttgcactatatcgaggaggggcccagtgttcagttggggtggagggacaggaagatctcagcgagaagtagacctgagccaggaggctggtattagggacctcctagaggaccatgtgcaggtggggccagggagggccaggacaagactgagagtatctgcgggcacaggcaggcaacactcagtgttggggaggcagctgctgcgctgggagtcaggagagctgcgttcggttctcgctcttccgctaatttgctgtgtgacctctttgagcctcgatattttcttgtgaaataaaggggagctaaataaactatctcaacggccacttctagggttaaaagtctctctaacgcctcctcccaggacgtgccaccccgggcgctcctacgcaagcattttctgggggcctgacactgctgcctccccgggctcactgaggagacaaggtggtccccatgagccgcattccgcaaaggggcagaggaggtctccgtgagcaggagcaggaggcacctgtttaaggtcacacagcaaaggggagaagaaaccagaggctgccaaactcctctgcaggagcccacacctgcctcacaccaggcaggcctgggggaagtccctcctctggcggaggaggcgaaacacagaggccagcccctcccctccagcccctgcaggtgtgtgtgttggggtgggatgggggcgctccccagcagctgcgtctctgccaggcacacacacctcgcggtaacacatatcccccacacaacccagcgcgctccacacacaggcatcttcctgtgcacgcgcgcacagaccccaacacctttccctctgttgccctcagcagggacgctcccacccccagtccccagcggacgcagtggcctcgggactcgtacacgcctccacgccccgccgcgtgacgtcacgtggggtagagccctgagacactaaatggcgggggggtggggggaggaaagggaaggcggcagagctccccgagccgggacagtcacttactctacaggcagtggggcccgacacagacagcgccgcccccgccagccagcctcgcacgccctcggaagcgcaggctcccggcgctgcgctggagggttccccggcaccccagcctcccgtccccagcccgctgcacctccgggccccccttacccttgagaggcaccgggagttgtcgcgggggggcctcgggaaattccccggacccctgtgccaggaggtgcccggttcgcccgctcttcaccccccgccccccccgagggcggtgcccgggggtgctgccccatggagcggggaggcgggcgccgtctgctccgggagccctgacccgagtcggagctgtgtgtcgcagccgccccgaccccccgccgatcatgcgccggcgcccctggctctccagtcccactgggctgtgagccccccactcccagcccgtcagggcctgcgcgccatgggcagcgcccaccctcgcccctggctgcggctccgaccccagccccagccgcggccagcgctctgggtgctcctgttcttcctactgctgctggctgctgccatgcccaggtgagccctcacctcatgctccgccctcctagagagttgggaccccggcctgcaggggcctctgatgctcttgctggggtagaggaccctggaagaagggagggacagggtcataggaaagtgctggtgcccaaccaaaggcagtgagctccctcataggagggggttggactggtgacaggatatgggcagtccagggagacaaggcagtgcccaggagcatgatggcaactaggaggggaggtgtgtgtggggggggagcaagtgccagtctggagttcctgccctgttgggggaatgcacccccacctcaggagtaagcacaagctgcctcactccttcctgcagctcagccccaggtaggggcaggggttgggaggctggaagggggtagcccaggtggggtcagtgagagactgcagctgcaagagctgtgtgtgtgtgtgtgtgtgtgtgtgtgttttcaatcgagcccaggatcccagcaaactgtgttggtaaacagtagtttgtatgtgtgcccatgtgtgcatgtgagagctcatgggtcaatgtatgttaagatgaacacatgtgtaaatgggagtgtctttgtgtgtgtgtctgtaactatgtctgatgaagtgtgcatgtgcgtaagtgtgctggggttgggtttgtgtgtccaccacaatgtgtgcagaagtgtgtctatgtgaatgtgactgaaacatatctgtgggagtgggcttgtggggaaccctgtgtgtatgggcatctattcctggggatgtgcataggaaatactgtggccacaagcatggtggatgtgtgtgcatttgcacgcttacctgtgtgggtgtgcagctagctgagtgagttgcactgggcaggatgattgtgaggaggtcaaggagagaggaatgtgtaggagtgtactgatgggcccaaccttcagaagcagaggttggaagagggagtgattatggccgttgggacagagtgtgtgttgttagatggggctctcctggtccccccaaaacacgtacccactccaccccatatgtctgcaggtcagcacccaatgacattctggacctccgcctccccccggagcccgtgctcaatgccaacacagtgtgcctaacattgccaggcctgagccggcggcagatggaggtgtgtgtgcgtcaccctgatgtggctgcctcagccatacagggcatccagatcgccatccacgaatgccaacaccaattcagggaccagcgctggaactgctcaagcctggagactcgcaacaagatcccctatgagagtcccatcttcagcagaggtagctgcccctcacccctgcccctgcctgccccatccagcatctccacctcagaatctattcccagccatttctaacccactgcctcaagctggcatcctcccatccccacacacccatctgttggccctgctgctctccttcctctttctgatctcaggccatgggggcagagaaattgggcaaatgtgtctccagactgggctgtgtaggctgagggtggcaggttccatgccttagaactggactcctgggtcatcctgccctatcactgacactgcatatctcaaacccaggaaatatctgtacctgtcgcttgcctctacttaccctctgcttagtaaaccacatttaggatggggaacccatgaggaagaacccagaatcagcatgaggaggggttgaggactttgcctaatcgtgcagatggaggcagcctggacaggtaaaaagtctgcccttctgcagaagctcttcccggcgggggacaccaccagcctgtgtgtcatccctgctgcctctggaaaaccctctctgaatcagaggtgctgggtgtgtgtgtgcctgtgtgcagtggctcctgccagcaggcctagtagtcctgtcaactcctcttcccctaacagctcagagaagctgcagtccattccctgtcctccctctccgacttctgggtgacccaccctgacaatcccagagtggcttgggctggggatggcaggaggttttgtcactcacttctgctcacacccacctcctggcctggtatctcaggccttgacttttgggaaggatgggggccctgcctctgggatgggcctagctgggtatctatctctcttctcagtggattgtgtgggacattgtgggcaccccgtagccctagctccttaggtcttgtccaattttcagtggaggtggagtgggagaggtacacatacaccttgtagccttgggtcttgaggcttcttggggagtcaggctgtacctcaaattcggaaaactcagaaagccgcggcccttcccttttcaccaggacccctgcttgcatcccctgggcccccaagtcaggggccaccccagcctccaccagcccagccccaggcctcccgccccgccccgccccgcccccgcgcgctccctcctccctcccccgcgcgccccgggcgctgtttctacccggccgagcccagcccgacgcgtgtggcggcatgcagccgcgaactaatccccgcgggccgcggcagacggctcccggccccagccgcgccgctctcagagggcaaccccctccctgccccgctctcccgccccctcccgccccaccgccgcccgggcccctccagccgtcca

SEQ ID NO: 49 - DNA, 8000 bp, Homo sapiens
  RARAlpha DNA region
gtaatcccagcactttgtggggccaaggcgggctgatcacttaagggtcaggagtgcaagaccagtctggccaacatggcgaaaccctgtctctactaaaagtacaaaaattcactgggtgccatggtacacacctataatcacagctacttgggaggctgagacaggagaattgcttgagcctgggaggtggaggttgcagtgagatgagatcatgccactgtactccagcctgggtgacagagtgagactctgtctcaaaaaaaaaaaaaaaatgtgtgaggtctaagctacccagagatcaaaaagttggcagagccttattctctctgcgccccctcttttgcattgctccctacctttactaagtgtccctttgaatatacatcaagccaggtgtggtggctcatgcctgtaatgccaacactttgggaggaggcagcagcgggaggattacttgaagtcaggagcttgagaccagcttgggcaatatagtgagacccccttacccttctctaccaaaaaaatcttaaaaatcagctgggcatggtggcatgcacctgtagtcctagctacttgggaagctgagatgggaggatgctttgagcccaggagttcaactcggcatgggctatgatcacaccactgcattacatcctgggtgacagagcgagaccctgtctctataaaagacagaaagcagatacaccaataagaatgagaagttgggaataagatgtgatttaggttttatatatatattatatacatattatatatattttatatatataatgtatattatatataatatacatattatatattatatacatattatttatatatatatatatatatattttttttttagatggtgtcttgctctgtcacccaggctggagcagtggcgcgatctctgttcactgcaagctccgcctcccgggttcacaccattctcctgcctcatcctgagtagctgggattacaggcacccgccaccatgcccggctaatttttttgtatttttagtaaagatggggtttcactgtgttagccaggatgatctcgatctcttgacctcgtgatccgcccgcctcggcctcccaaagtgctgggattagaggcttgagccaccatgcctggccggttatatattttttaaatatcttgagacgtgttaccttgactgctgagggaggacaaaagagatccaagattgaaattttatttacatttgtatctgtgcctagcaattagtatagtgcctaatacctggtaggtactcaatagttgtttaacagataagtgaaccaaataagatactgcctttccctcagtggtgagaacagttctaagctgaaggtaaacgttggtggtacttttgcacagaggtggagcaggcccctgcataagaagggtgctctgtcttacaaaatgtgtcttggcacagttttggagcatttattagcctcacagatgcattccaatcactccctctgatgtagtggcattttgcttgaggcaacttccatgttgctggtgggacgctttggctatgaggggaagagtcttatctacaggctgtgaagagggctgcctgtctggatgtcactcagtgtggctagattggccagaagctgggtctgtaggcaagggtttagggaggagcccacctttgatgtccctctttttaacttggggcaagttgtgatctcaggttcttcctgaaaagataaggtactttctaagataccttctcttaagctacaaacagtgcaggtcttcctcttagaagaggtgtgttccgtaaggggagaaggtgtgccctctggcctccccttgattttcaaccccagccccaaggcactggtagtggaagttaattgggtgcaaggggcttgggtaaaggctgaagactggtgaaaggagagagccagcttcgttttttaaaaagctctaattgagttctgaatcagcctcactccctttgatccctcctctgtttcccacactgcctgaaagagctagatcagcactttataacaagggtgcgcctgctccccgcaccgccccccgctcctgacgctctctgctgccgccatccactgcttgtctccttcagttaagataatgaagatgctgagagataaaatgtgtatttaataagggtgggggtagggaggcggaaaggggcaaccagggccttccctagtttggggcctcgccaagacttaagctgaaagcaaagctagagtaagacttggccccagggtgtgggggtttctgggcctcaatccagctgacttcactgcctgctgtttgccctaaattcccccacctccaccctctcacccccacactgctttcttcttgctcccaatgggaaaaaggactcttgcttcaactttcttttcagctggtaacttggcctttggtgcggtgccccccaccccagtgtcagctgcaaggtcgggctagggcagccccctctagcctaggcctgtagaagctgtgggttcacagtggggggtgaaaaagctggagttaatgaagaccagctgctagtctgggttctgcctacagctagctggaggccagcctttggtggtggttgatgggggggtggggtgacctccatccatgggcacaaggatatcctgtggcagctaggcttgtgcttctctctgagcttagttttatccttagtttataagagagtatctgtttccttcccctactcctctcctccccaaataacactcagctcttattgaactttataactgtagtggtggcccagctgagatctgaggatctccaaacccttttgtaaacactcattaactgcctgggaagaacgaagagccctgtttaacagctgggagagaactgaggcacattagggtgaaaggtttgacctaaactcaccccatggactccaccctggggccttccatccttagtccagctcctttactcagttcttcctgcaagacagaggtggaggcattgctggtgtcttctccccttctagggagaggccatgccctctcccctcaagtctgtcgctgacttcctctggcccttcccctcatgacgttttccctgctctgctgctagagatatgtgcaactctccttgcaaaatgttctcaatccacaacttcagaattcttatccagctttctcttgcccatctagcccaccaaacctgaattctcccttttgagctactgagaagactggctatactaaactgaatcagtgaccttgaagttgatctgttgtattgaggtccctctgcccctatatttatcctaaatggtatctaggaaaatattctctctttaacttgtttattctggtcataaagattcgggacattactgagggctgggtaggggcactagttgacctctaggttctacgtcctgttcttccactgacaattaccatttccagtaataggtgtaacccaaagttcttttttgtttttttctccttccctaaccccacctgtttatactcttgcctatatatgtgtatttgcagaccccacacattcccagatacacatacattggtcctacatgtttcccctgtttccagctgccatctaagtgagcctgcactttttacatgctgaaaatcttttttcaccctctatggcctcctaggtccttgggaataattgggagggatgaagggactcctgcagtccatgaggaagaggacagaagtgagcgcccaaccaaaatgaatgctccctttccaaaatctctagctgaatgccttagcaggagtggcagggaaagagatcttaaccccacctccagcaccaaaacctaaatccatctgctcagggcaggcaggaagaaatgtctattttctcctgtttcaggtcatcctatcctcgagttatccatatgctgtgtatttgagacagggaactgttcctgtccccagccgatgaccagacgcccatctttcttccagtgggttacttctcccccatcagtccttcgctctgcgcccgctgacccagtcggggctggctggtggaagagggcggggagagccttctagttggcgagtgggctttaggacccaacgggaacccgtgcctcttgcagcagcctaacccagaagcaggggggaatcctgaatcgagctgagagggcttccccggttctcctgggaaccccatcggccccctgccagcacacacctgagcaggtaggaccatgcacaccccttcccaattctttggccgcctttgaccccggcctctgcttctgatctagccatttccttctcgccagttctggctttccccatccctcctctcccgtcgctcactgtagtcctgggaggtgggagcctgggaacagtggggtgtctgaaagctcagagccctctgcaccctccagcacccttcctgcatttctcctgaggcagttcccgttctctagataagagtggggagtagggggaaggtctgcccaggccccacgcccttgcaccctcctttgcggtattgcctaataccccttcccccacctcacctagactggagaattggcaagtgctgaggtaacagagagaggagagagccagagagggacctgagctttcccaagggatttctttcttgcttcctcacccaaagagctcaggactttgcccctactccctacacaccttgtaggggaactaatgtccccttccaggttcatctcacacttgggtggctaaaggctttgctagagggtagtggaggttgtgagcattttggtgagcttttcctctaggaaagttctttcagcttagagcccccatccatgtcaacatttcaacacctgctgcagaagccccccgaccccacctgtgtgtgtgtgtgtgtagggacaatgtggttgtaagatctagaagctttggatagaggtggctgcatgcattgaaggggagggataccagagtgtggcccaggccagaacaggctcagtccagatggctagggaagggggtgtggaccagattgaggaggtcacccagccttgccagctccctgagcttcttggctttcagaatctcaggctcctggtgtttctctagagctacaaggtccccaagcttctgccccgctacaggctctctgggagagggctagatggcacctactgtttacagtggcttatttttctctcctgtggtgggcagacaggccctgctctctgaagatctagggcgtgtcattctggtttggtgggtttacaggaagggtgtacaccttgtctgacttaccatgtgtgagtgtgtgtcttgcttctgggggtagtagactggggaggggggtgatagaagagactggtcacactgagggaactcctctagcaatcaatgtatgataggaatgggtggcatgtccccatctggtagctctgtctaggtgtggggtgtgctctgcattcttccatggctccccgggtcccctaaaggttggttttagaggacagggtggataatgaattggcttcagacttcgttctggtcttgatccaacaaggaaagtagagattctgactgtgagcagtgaactaggaatcagaggcctgaactggagacttagactggttacttaactatatgtgtttatataggtcttcccctttgctaagccttagtttctccatttgtaaaaatggctatcagtagcaagcagggacattgccactaaaaccacagagacaatgtatcgtaaagctcactgcaaacggtagtaatggctgggggaatctaggatggaatcctcttcctaaatctaggtcaatctctccgcctttagttttcttcccccatcctttgcccatctgggggatggaggaactaggcagggctattgccccggttactgcagaattcccctaatgcaggttgtagctgttaccagagcacctgtggtcacagagtacagatctctccaccaggcatttcttcctctggggccccatacttgctcccctgagccatgtctccaccttccttctgccatccctgcttggcgtccttgtctctctctcccctgcccagtggcctccttgtctggctcactgggcaggagccctaatcggattcgacagctgagatatgtgtggcggctaagagcaggcaggagggaagccagtgggaggatggtatgaggactgcccctggcactgccccggcctcccaagcaccaccttgggcctgctgatttcttacttggcttggccccaaggaaggggtgccagaggaagctcaggaatgaacaggctggggtctggagactaggagaggaggaaacacaccaaggtcctcaggacctggttgatattcgatgaccttaatgtcagtgtcactgactctgacaaggaggaaaggacagcgatgagccaggctgacccccgccaccccattcccatagccccactttcttctctcttcttgttcagaaatgtttcactttgcctcatctagcccctttggccagtaggtcacatctgggagcttctgggggtgccacatgtctgcctcaatctgggctgtttcctcccctccaagatatttcactgtctctgggctgggcgccaggactcctgggtttccctgcctgtggtgcagggctcccctgcagggctccagagagtcgcttagctggtttccttcctcctttgtggggagggccttcccctagggctgggaggtgtcaggaatcccaggtgggacaggggtgggggcacagtagcctctggcactgtagcaccccagcttctccctcctagcctctgactcagcttgggggcagcacatgcccagcctggcaggcatggcctattcttgcctccaggcttctctctttacagaagcctacatctcctgctctcattttttagtttttttccccatgatatctgcaggtggctagtgaaatccctggttttggggagcaagggactaacataattgaggctggggcactattttgtcttccctccagttgggccaggatgaaggagactgtcacaaacttccccaagccaattccttctgccctcaaaagaccagtcagtggagccaggccctgccctttgggtgcccacaatgtggttggcactgatttgtcagaaattgggaaggtttccctctagaccgcttgcctcctttctcctcctcccgcagggtaggggcagctctgtgattgcaggcacttatgggcaggcctccccctaggaaaggggtgtgggatgcccccaggatggccaagttggcgctagccccaggctggagtgctggacatgattctgacctgggagttagtgggggtaaggcagggcatgggcctccctgcccccctcatttcctgctctttgggcagctgtgggcgagaaaggctgcagggactatttttagttggtcagtcccctggcaggagggacgtgcagaaaagcacggggcaggcagaggggctggccaaggtgagggtaagcaggcaggcagaggagtgaggaagggccagaggggcagaagacagcaaggaggcatggggagcaggggcaggaccttacccagaggggcatgacccaggtagatgggccgggctggactgggtggaagagtcctggcaccctacagggttttctttatccagctggactcagagctggaaggggttttagaggctacctaggcaatcccttccccttgcagagaaggaaactgaggcctagagagaacactcaatcagtcattggctgtgttgagacctgaacccaggccctccctttgcaccatttttctgttgtcatggttcctcaccacagctcagtgtgcttcccttccctactgcagcaaggaggaccacgagccaggcacacctggtccattttcccatctgcttttttgtgcagggtctctgaaaatcccagcttggtttgtttataagtctccatgtggaaagag

SEQ ID NO: 50 - DNA, 27 bp, Artificial Sequence
  bisulfite converted DNA PCR primer
gagggattagattttgggtttttgtag

SEQ ID NO: 51 - DNA, 30 bp, Artificial Sequence
  bisulfite converted DNA PCR primer
aataaccctcacccaaatatccacctaatt